(19) United States
(12) Patent Application Publication
(10) Pub. No.: US 2023/0398077 A1
(43) Pub. Date: Dec. 14, 2023

(54) SILICASOME NANOCARRIER FOR METAL-BASED DRUG DELIVERY

(71) Applicant: The Regents of the University of California, Oakland, CA (US)

(72) Inventors: Andre E. Nel, Sherman Oaks, CA (US); Huan Meng, Los Angeles, CA (US); Xiangsheng Liu, Los Angeles, CA (US)

(73) Assignee: The Regents of the University of California, Oakland, CA (US)

(21) Appl. No.: 18/250,727

(22) PCT Filed: Oct. 28, 2021
(86) PCT No.: PCT/US2021/057122

§ 371 Date: Apr. 26, 2023

Related U.S. Application Data
(60) Provisional application No. 63/108,172, filed on Oct. 30, 2020.

(51) Int. Cl. A61K 9/16; A61K 9/51; A61K 9/50; A61K 47/64; A61K 47/68; A61K 45/06; A61K 39/395; A61P 35/00; A61K 31/282; A61K 33/243; A61K 47/69

(52) U.S. Cl. CPC A61K 9/167; A61K 9/5115; A61K 9/5015; A61K 47/6425; A61K 47/6803; A61K 45/06; A61K 39/3955; A61K 9/5089; A61P 35/00; A61K 31/282; A61K 33/243; A61K 47/6929; A61K 2039/505

(57) ABSTRACT

In various embodiments, drug delivery vehicles that contain one or more metal-based therapeutic agents are provided. In certain embodiments, the drug delivery vehicle comprises: a silica nanoparticle comprising one or more cavities disposed within the nanoparticle and an outside surface where the one or more cavities are in fluid communication with the outside surface of the nanoparticle; a metal-based (e.g., platinum-based) chemotherapeutic drug disposed on the surface of the nanoparticle and/or within the one or more cavities of the nanoparticle where the drug comprises a cationic, metal-based drug; and a lipid bilayer disposed on the surface of the nanoparticle where the lipid bilayer fully encapsulates and seals the nanoparticle.

### CROSS-REFERENCE TO RELATED APPLICATIONS

This application claims benefit of and priority to U.S. Ser. No. 63/108,172, filed on Oct. 30, 2020, which is incorporated herein by reference in its entirety for all purposes.

### STATEMENT OF GOVERNMENTAL SUPPORT

This invention was made with government support under Grant Number CA198846, awarded by the National Institutes of Health. The government has certain rights in the invention.

### INCORPORATION BY REFERENCE OF SEQUENCE LISTING PROVIDED AS A TEXT FILE

[Not Applicable]

### BACKGROUND

Pancreatic ductal adenocarcinoma (PDAC) is a highly lethal cancer with 5-year survival of ˜8%^{[1]}. As surgery is only suitable for ˜15% of PDAC patients upon diagnosis, the best clinically available option is to use chemotherapy in the majority of PDAC patients^{[2]}. This can be accomplished by the use of a gemcitabine (GEM)/Nab-paclitaxel combination or a four-drug regimen, FOLFIRINOX, which includes folinic acid, 5-fluorouracil, irinotecan, and oxaliplatin^{[3]}. While the recent breakthrough of a modified FOLFIRINOX regimen that reduces the irinotecan dose has yielded promising results in a phase 3 study, the use of the alternative option is still limited due to the occurrence of toxic side effects and chemo-resistance^{[3c]}. In order to develop a nanocarrier with reduced irinotecan side effects, we have previously developed mesoporous silica nanoparticles (MSNP), coated with a lipid bilayer for safer and more efficacious irinotecan delivery^{[4]}. The success of this carrier, also known as a “silicasome”, was largely attributed to the ability of a proton-generating trapping agent for remote import of the weak-basic molecule, irinotecan, into the large packaging space of the porous interior^{[4]}. This resulted in improved irinotecan delivery, which was accompanied by a significant improvement in treatment efficacy for PDAC, prolonged survival, and reduced toxicity compared to the free drug or a liposomal carrier, ONIVYDE®^{[4b]}.

Oxaliplatin is another active pharmaceutical ingredient (API) of the FOLFIRINOX regimen that is very potent but exhibits major, and dose-limiting toxicity (e.g., bone marrow)^{[5]}. In addition, oxaliplatin is known to exert immunogenic effects that could be potentially useful to supplement its chemotherapeutic effects^{[6]}. However, since Pt drugs are coordination compounds, it is not possible to do remote loading in MSNPs to obtain high loading capacity, which has limited the utility of this carrier for Pt drugs. It would be of great advantage to develop MSNPs carriers for efficient Pt drug loading and delivery from the perspective that ˜50% of all cancer patients undergoing chemotherapy receive at least one type of Pt-based treatment^{[7]}.

Pt-based antineoplastic molecules (FIG. 7) are coordination compounds, with the generalized chemical structure of cis-[PtA₂X₂] as shown in in FIG. 1. A₂ represents two monodentate or one bidentate ligands with nitrogen donor atoms, while X₂ are comprised of two monodentate or one bidentate anionic ligand(s)^{[8]}. When delivered to a tumor environment, Pt drugs provide DNA crosslinking through the formation of platinum-DNA adducts (a.k.a. alkylation)^{[9]}, in addition to the triggering of additional biological responses such as STAT signaling, induction of endoplasmic reticulum stress, and immunogenic cancer killing, etc.)^{[6]}. This enables the Pt drugs, including their encapsulated versions^{[10]}, to be used for the treatment of wide variety of cancers cancer, including PDAC^{[7b]}. However, since remote loading of these drugs in MSNPs is not an option, other attempts at encapsulation have been tried with limited success, yielding a Pt:MSNP with low (0.87 wt %) loading capacity (LC %) in an unmodified MCM-41 MSNP^{[11]}. This sparked additional attempts to improve the LC % to 10-20 wt % by attaching a carboxy (—COOH) group to the MSNP surface, allowing coordination binding to cisplatin^{[12],[13]}. Similar attempts were undertaken to improve oxaliplatin loading, providing an agent with a broader spectrum of activity and reduced cross-resistance compared to cisplatin^{[9]}. Specifically, COOH-modified MSNP was used for complexation to cis-dichloro(1,2-diamminocyclohexane) platinum(II) (a.k.a. DACHPtCl₂), which is the precursor to oxaliplatin^{[14]}. This improved the oxaliplatin loading capacity to 16.1 wt %^{[14]}. The downside of a post-grafting carboxy modification, however, is that it interferes in coating with a lipid bilayer, which is critical for colloidal stability and systemic biodistribution^{[15]}. This likely reflects the reason why the studies were only undertaken in the tissue culture conditions, rather than in vivo.

### SUMMARY

Herein, we report an intravenous (IV) injectable tailored-designed silicasome carrier for Pt drug encapsulation. This was achieved by introducing active Pt drugs that can be efficiently loaded under a carefully designed complexation conditions, making use of weak basic pH conditions. Instead of using a post-grafting approach, we took advantage of the pH-dependent properties of the surface silanol groups for electrostatic and coordination binding of DACHPt, followed by applying a uniform lipid coat to seal the MSNP pores. This strategy, which can also be adapted to load other types of Pt payloads, led to high-loading and colloidally stable nanocarriers. The availability of a DACHPt silicasome allowed us to perform efficacy and safety studies in an orthotopic Kras PDAC model. It was also possible to use the silicasome-encapsulated DACHPt to test its immunogenic effects for the development of a chemo-immunotherapy approach in combination with an anti-PD-1 antibody in an orthotopic PDAC survival study.

Accordingly, various embodiments contemplated herein may include, but need not be limited to, one or more of the following:
Embodiment 1: A drug delivery vehicle for the delivery of a metal-based drug, wherein said drug delivery vehicle comprises:
   a silica nanoparticle wherein:
      i) said silica nanoparticle is a solid silica nanoparticle; or:
      ii) said silica nanoparticle comprises one or more cavities disposed within said nanoparticle and an outside surface where said one or more cavities are in fluid communication the outside surface of said nanoparticle;
   a metal-based drug disposed on the surface of said nanoparticle and/or within said one or more cavities; and
   a lipid bilayer disposed on the surface of said nanoparticle where said lipid bilayer fully encapsulates and seals said nanoparticle.
Embodiment 2: The drug delivery vehicle of embodiment 1, wherein said metal-based drug comprises a metal selected from the group consisting of platinum, palladim, gold, ruthenium, titanium, technetium and rhenium galdolinium, cobalt, lithium, bismuth, iron, calcium, lanthanum, gallium, tin, arsenic, rhodium, copper, zinc, aluminum, lutetium, vanadium, and manganese.
Embodiment 3: The drug delivery vehicle of embodiment 2, wherein said metal-based drug comprises a metal-based drug selected from the group consisting of a palladium complex drug, a gold complex drug, a ruthenium complex drug, and a titanium complex drug.
Embodiment 4: The drug delivery vehicle of embodiment 3, wherein said metal-based drug comprises a platinum based chemotherapeutic drug disposed on the surface of said nanoparticle and/or within said one or more cavities of said nanoparticle where said drug comprises a cationic, activated Pt drug.
Embodiment 5: The drug delivery vehicle of embodiment 4, wherein said metal-based drug comprises a drug selected from the group consisting of 1,2-diaminocyclohexane)platinum(II) (DACHPt), diaminoplatinum(II) (DAPt), ethylenediamine platinum (EDAPt), a cationic form of carboplatin, a cationic form of nedaplatin, a cationic form of heptaplatin, a cationic form of lobaplatin, a cationic form of iproplatin, a cationic form of tetraplatin, a cationic form of satraplatin, a cationic form of triplatin tetranitrate, a cationic form of phenanthriplatin, a cationic form of picoplatin, and a cationic form of setraplatin.
Embodiment 6: The drug delivery vehicle of embodiment 4, wherein said metal-based drug comprises a drug selected from the group consisting of 1,2-diaminocyclohexane)platinum(II) (DACHPt), diaminoplatinum(II) (DAPt), and ethylenediamine platinum (EDAPt).
Embodiment 7: The drug delivery vehicle of embodiment 6, wherein said platinum based chemotherapeutic drug comprises 1,2-diaminocyclohexane)platinum(II) (DACHPt).
Embodiment 8: The drug delivery vehicle of embodiment 6, wherein said platinum based chemotherapeutic drug comprises diaminoplatinum(II) (DAPt).
Embodiment 9: The drug delivery vehicle of embodiment 6, wherein said platinum based chemotherapeutic drug comprises ethylenediamine platinum (EDAPt).
Embodiment 10: The drug delivery vehicle of embodiment 3, wherein said metal-based drug comprises a palladium complex.
Embodiment 11: The drug delivery vehicle of embodiment 10, wherein said metal-based drug comprises trans-[PdCl₂(2-dqmp)] (2-dqmp=diethyl-2-quinolmethylphosphonate or glycoconjugated Pd(II) complex, ([PdCl₂(L) where L=2-deoxy-2-[(2-pyridinylmethylene) amino]-a-D-glucopyranose).
Embodiment 12: The drug delivery vehicle of embodiment 3, wherein said metal-based drug comprises a gold complex.
Embodiment 13: The drug delivery vehicle of embodiment 12, wherein said gold complex comprises an Au(III) complex with multidentate ligands.
Embodiment 14: The drug delivery vehicle of embodiment 13, wherein said Au(III) complex is selected from the group consisting of [Au(en)Cl₂][Cl], [Au(dien)Cl][Cl₂], [Au(cyclam)][ClO₄]₂Cl, [Au(terpy)Cl][Cl₂], and [Au(phen)Cl₂][Cl].
Embodiment 15: The drug delivery vehicle of embodiment 12, wherein said gold complex comprises an Au(III) complex that contains a functionalized bipyridine ligand of the general formula [Au(N—N)Cl₂][PF₆], where N—N is elected from the group consisting of 2,2′-bipyridine; 4,4′-dimethyl-2,2′-bipyridine, 4,4′-dimethoxy-2,2′-bipyridine, and 4,4′-diamino-2,2′-bipyridine.
Embodiment 16: The drug delivery vehicle of embodiment 12, wherein said gold complex comprises an Au(III) complex of the type [Au(dach)(pn)]Cl₃ where dach is cis-, or trans-1,2-; and S,S-1,2-diaminocyclohexane and pn=1,3-diaminopropane.
Embodiment 17: The drug delivery vehicle of embodiment 3, wherein said metal-based drug comprises a ruthenium complex.
Embodiment 18: The drug delivery vehicle of embodiment 17, wherein said ruthenium complex is selected from the group consisting of KP1019, NAMI-A, RAPTA-C, and RAPTA-T.
Embodiment 19: The drug delivery vehicle of embodiment 3, wherein said metal-based drug comprises a titanocene.
Embodiment 20: The drug delivery vehicle of embodiment 19, wherein said metal-based drug comprises a titanocene selected from the group consisting of titanocene X, and titanocene Y.
Embodiment 21: The drug delivery vehicle according to any one of embodiments 1-20, wherein said nanoparticle is a solid nanoparticle.
Embodiment 22: The drug delivery vehicle according to any one of embodiments 1-20, wherein said nanoparticle comprises one or more cavities disposed within said nanoparticle and an outside surface where said one or more cavities are in fluid communication the outside surface of said nanoparticle.
Embodiment 23: The drug delivery vehicle according to any one of embodiments 1-22, wherein said drug is disposed on the surface of said nanoparticle.
Embodiment 24: The drug delivery vehicle according to any one of embodiments 1-23, wherein said drug is disposed within a cavity in said nanoparticle.
Embodiment 25: The drug delivery vehicle according to any one of embodiments 22-24, wherein said nanoparticle comprise a single cavity.
Embodiment 26: The drug delivery vehicle of embodiment 25, wherein said nanoparticle comprises a nanobowl.
Embodiment 27: The drug delivery vehicle of embodiment 25, wherein said nanoparticle comprises a hollow nanosphere.
Embodiment 28: The drug delivery vehicle according to any one of embodiments 1-24, wherein said nanoparticle comprises a plurality of cavities.
Embodiment 29: The drug delivery vehicle according to any one of embodiments 1-28, wherein said drug is disposed on the surface of said nanoparticle.
Embodiment 30: The drug delivery vehicle according to any one of embodiments 1-23, wherein said drug is disposed within a cavity in said nanoparticle.
Embodiment 31: The drug delivery vehicle according to any one of embodiments 1-30, wherein said nanoparticle comprises a mesoporous silica nanoparticle (MSN), a mesoporous organosilica nanoparticle (MONs), a periodic mesoporous organosilica (PMO) nanoparticle, a solid silica nanoparticle, or a silica thin layer.
Embodiment 32: The drug delivery vehicle of embodiment 31, wherein said nanoparticle comprises a mesoporous silica nanoparticle (MSN).
Embodiment 33: The drug delivery vehicle according to any one of embodiments 1-32, wherein said nanoparticle comprises an inorganically doped silica.
Embodiment 34: The drug delivery vehicle of embodiment 33, wherein said nanoparticle comprises a calcium-, iron-, manganese-, or zirconium-doped silica.
Embodiment 35: The drug delivery vehicle according to any one of embodiments 1-34, wherein said nanoparticle comprises an imine-doped silica.
Embodiment 36: The drug delivery vehicle according to any one of embodiments 1-35, wherein said nanoparticle comprises a mesoporous silica/hydroxyapatite (MSNs/HAP) hybrid nanoparticle.
Embodiment 37: The drug delivery vehicle according to any one of embodiments 1-36, wherein said nanoparticle comprises a cleavable silsesquioxane, or a bridged silsesquioxane (BS).
Embodiment 38: The drug delivery vehicle according to any one of embodiments 1-32, wherein said nanoparticle is undoped and silica comprising said nanoparticle is not functionalized with a moiety other than a silanol group.
Embodiment 39: The drug delivery vehicle according to any one of embodiments 1-38, wherein said nanoparticle is functionalized with silanol groups.
Embodiment 40: The drug delivery vehicle according to any one of embodiments 1-39, wherein said lipid bilayer comprises a phospholipid, and cholesterol (CHOL) and/or a cholesterol derivative.
Embodiment 41: The drug delivery vehicle of embodiment 40, wherein said lipid bilayer comprises a phospholipid and cholesterol (CHOL).
Embodiment 42: The drug delivery vehicle according to any one of embodiments 40-41, wherein said phospholipid comprises a saturated fatty acid with a C14-C20 carbon chain, and/or an unsaturated fatty acid with a C14-C20 carbon chain, and/or a natural lipid comprising a mixture of fatty acids with C12-C20 carbon chains.
Embodiment 43: The drug delivery vehicle of embodiment 42, wherein said phospholipid comprises one or more phospholipids selected from the group consisting of distearoylphosphatidylcholine (DSPC), phosphatidylcholine (DPPC), 1,2-dimyristoleoyl-sn-glycero-3-phosphocholine (DMPC), 1,2-dioleoyl-sn-glycero-3-phosphocholine (DOPC), 1,2-distearoyl-sn-glycero-3-phospho-rac-glycerol (DSPG), 1,2-dipalmitoyl-sn-glycero-3-phosphoglycerol (DPPG), 1,2-dieicosenoyl-sn-glycero-3-phosphocholine, and diactylphosphatidylcholine (DAPC), dipalmitoyl phosphatidylethanolamine, Dioleoyl-N-Glutaryl-L-α-phosphatidylethanolamine (DOPE-Glu), 1-Palmitoyl-2-oleoyl-N-Glutaryl-L-α-phosphatidylethanolamine (POPE-Glu), Dipalmitoyl-N-Glutaryl-L-α-phosphatidylethanolamine (DPPE-Glu), and Distearoyl-N-Glutaryl-L-α-phosphatidylethanolamine(DSPE-Glu).
Embodiment 44: The drug delivery vehicle of embodiment 42, wherein said phospholipid comprises a natural lipid selected from the group consisting of egg phosphatidylcholine (egg PC), and soy phosphatidylcholine (soy PC).
Embodiment 45: The drug delivery vehicle of embodiment 42, wherein said phospholipid comprises distearoylphosphatidylcholine (DSPC).
Embodiment 46: The drug delivery vehicle according to any one of embodiments 40-45, wherein said lipid bilayer comprises an mPEG phospholipid with a phospholipid C14-C18 carbon chain, and a PEG molecular weight ranging from about 350 Da to 5000 Da.
Embodiment 47: The drug delivery vehicle of embodiment 46, wherein said lipid bilayer comprises dipalmitoyl phosphatidylethanolamine grafted poly(ethylene glycol) (PE-PEG).
Embodiment 48: The drug delivery vehicle of embodiment 47, wherein said PE-PEG comprises PE-PEG_{2K}.
Embodiment 49: The drug delivery vehicle of embodiment 47, wherein said PE-PEG comprises PE-PEG_{5K}.
Embodiment 50: The drug delivery vehicle according to any one of embodiments 45-49, wherein said lipid bilayer comprises DPSC, cholesterol, and PE-PEG.
Embodiment 51: The drug delivery vehicle of embodiment 50, wherein the ratio of DPSC:cholesterol:PE-PEG ranges from 40-90% DSPC:10%-50% Chol:1%-10% PE-PEG (molar ratio).
Embodiment 52: The drug delivery vehicle of embodiment 51, wherein said bilayer comprises DSPC:cholesterol:PE-PEG at a molar ratio of about 3:2:0.15 for DSPC, cholesterol, and PE-PEG, respectively.
Embodiment 53: The drug delivery vehicle according to any one of embodiments 40-52, wherein said lipid bilayer comprises a cholesterol derivative selected from the group consisting of cholesterol hemisuccinate (CHEMS), lysine-based cholesterol (CHLYS), and PEGylated cholesterol (Chol-PEG).
Embodiment 54: The drug delivery vehicle of embodiment 53, wherein said lipid bilayer comprises CHEMS.
Embodiment 55: The drug delivery vehicle of embodiment 54, wherein said bilayer comprises CHEMS ranging from about 5% (mol percent) up to about 30% total lipid.
Embodiment 56: The drug delivery vehicle of embodiment 55, wherein said bilayer comprises about 10% or about 20% CHEMS or about 30% CHEMS or about 40% CHEMS.
Embodiment 57: The drug delivery vehicle of embodiment 53, wherein said cholesterol derivative is used in place of said cholesterol.
Embodiment 58: The drug delivery vehicle according to any one of embodiments 1-39, wherein said drug delivery vehicle has an average hydrodynamic diameter ranging from about 30 nm up to about 300 nm, or from about 40 nm up to about 200 nm, or from about 50 up to about 100 nm, or from about 60 nm up to about 90 nm, or from about 70 nm up to about 90 nm, or from about 80 nm up to about 90 nm by DLS.
Embodiment 59: The drug delivery vehicle of embodiment 58, wherein said drug delivery vehicles have an average hydrodynamic diameter ranging from about 79 nm up to about 86 nm by DLS.
Embodiment 60: The drug delivery vehicle according to any one of embodiments 1-59, wherein said drug delivery vehicle has an average hydrodynamic diameter ranging from about 30 nm up to about 300 nm, or from about 50 nm up to about 250 nm, or from about 70 nm up to about 200 nm, or from about 90 nm up to about 150 nm, or from about 110 nm up to about 150 nm by cryoEM.
Embodiment 61: The drug delivery vehicle of embodiment 60, wherein said drug delivery vehicle has an average hydrodynamic diameter ranging from about 136 nm up to about 139 nm by cryoEM.
Embodiment 62: The drug delivery vehicle according to any one of embodiments 1-61, wherein a plurality of said drug delivery vehicles, in suspension, has a PDI ranging from about 0.050 up to about 0.20, or from about 0.050 up to about 0.1.
Embodiment 63: The drug delivery vehicle of embodiment 62, wherein a plurality of said drug delivery vehicles, in suspension, has a PDI of about 0.076.
Embodiment 64: The drug delivery vehicle according to any one of embodiments 1-63, wherein said lipid bilayer ranges in thickness from about 5 to about 12 nm.
Embodiment 65: The drug delivery vehicle of embodiment 64, wherein said lipid bilayer ranges in thickness from 6 nm to about 7 nm.
Embodiment 66: The drug delivery vehicle according to any one of embodiments 1-65, wherein said vehicle entraps at least about 50%, or at least about 60%, or at least about 70% said metal-based drug.
Embodiment 67: The drug delivery vehicle of embodiment 66, wherein said drug delivery provides an EE % of at least about 40%, or at least about 50%, or about 53%.
Embodiment 68: The drug delivery vehicle according to any one of embodiments 66-67, wherein said drug delivery provides an LC % of at least about 15 wt %, or at least about 20 wt %, or about 21 wt %.
Embodiment 69: The drug delivery vehicle according to any one of embodiments 1-68, wherein said drug carrier comprises an additional therapeutic agent disposed inside of the nanoparticle or associated with the lipid bilayer.
Embodiment 70: The nanoparticle of embodiment 69, wherein said drug carrier comprises an additional therapeutic agent disposed inside of the nanoparticle.
Embodiment 71: The nanoparticle of embodiment 69, wherein said drug carrier comprises an additional therapeutic agent disposed inside of the nanoparticle or disposed within the lipid bilayer or conjugated to the lipid bilayer.
Embodiment 72: The drug delivery vehicle of embodiment 69, wherein said additional therapeutic agent comprises a second metal-based drug.
Embodiment 73: The drug delivery vehicle of embodiment 72, wherein said second metal-based drug comprises a metal selected from the group consisting of platinum, palladim, gold, ruthenium, titanium, technetium and rhenium galdolinium, cobalt, lithium, bismuth, iron, calcium, lanthanum, gallium, tin, arsenic, rhodium, copper, zinc, aluminum, lutetium, vanadium, and manganese.
Embodiment 74: The drug delivery vehicle of embodiment 73, wherein said second metal-based drug comprises a metal-based drug selected from the group consisting of a palladium complex drug, a gold complex drug, a ruthenium complex drug, and a titanium complex drug.
Embodiment 75: The drug delivery vehicle of embodiment 74, wherein said second metal-based drug comprises a metal-based drug selected from the group consisting trans-[PdCl₂(2-dqmp)] (2-dqmp=diethyl-2-quinolmethylphosphonate, glycoconjugated Pd(II) complex, [PdCl₂(L)] (L=2-deoxy-2-[(2-pyridinylmethylene) amino]-a-D-glucopyranose, [Au(en)Cl₂][Cl], [Au(dien)Cl][Cl₂], [Au(cyclam)][ClO₄]₂Cl, [Au(terpy)Cl][Cl₂], [Au(phen)Cl₂][Cl], [Au(N—N)Cl₂][PF₆] where N—N is 2,2′-bipyridine, 4,4′-dimethyl-2,2′-bipyridine, 4,4′-dimethoxy-2,2′-bipyridine, or 4,4′-diamino-2,2′-bipyridine, [Au(dach)(pn)]Cl₃ where dach is cis-, or trans-1,2-, or S,S-1,2-diaminocyclohexane and pn is 1,3-diaminopropane, KP1019, NAMI-A, RAPTA-C, RAPTA-T, titanocene X, and titanocene Y.
Embodiment 76: The drug delivery vehicle of embodiment 69, wherein said additional therapeutic agent comprises an agent selected from the group consisting of doxorubicin, irinotecan, topotecan, 10-hydroxycamptothecin, belotecan, rubitecan, vinorelbine, LAQ824, vinblastine, vincristine, homoharringtonine, trabectedin, anthracyclines, epirubicin, pirarubicin, daunorubicin, rubidomycin, valrubicin, amrubicin, mitoxantrone, cyclophosphamide, mechlorethamine, temozolomide, 5-fluorouracil, 5′-deoxy-5-fluorouridine, gemcitabine, capecitabine, pazopanib, enzastaurin, vandetanib erlotinib, dasatinib, nilotinib, sunitinib, osimertinib, palbociclib, and ribociclib.
Embodiment 77: The drug delivery vehicle of embodiment 69, wherein said additional therapeutic agent comprises an inhibitor of the indoleamine 2,3-dioxygenase (IDO) pathway (IDO pathway inhibitor).
Embodiment 78: The drug delivery vehicle of embodiment 77, wherein said drug carrier, when administered systemically, delivers an amount of an IDO pathway inhibitor to partially or fully inhibit the IDO enzyme or IDO pathway at a cancer site.
Embodiment 79: The drug delivery vehicle according to any one of embodiments 77-78, wherein said IDO pathway inhibitor comprises an inhibitor of the IDO enzyme.
Embodiment 80: The drug delivery vehicle according to any one of embodiments 77-79, wherein said IDO pathway inhibitor comprises an inhibitor of the IDO pathway downstream from said IDO enzyme.
Embodiment 81: The drug delivery vehicle according to any one of embodiments 77-80, wherein said IDO pathway inhibitor comprises an agent selected from the group consisting of of D-1-methyl-tryptophan (indoximod, D-1MT), L-1-methyl-tryptophan (L-1MT), a mixture of D-1MT and L-1MT, 1-methyl-L-tryptophan (L-1MT), methylthiohydantoin-dl-tryptophan (MTH-Trp, Necrostatin), β-carbolines (e.g., 3-butyl-β-carboline), Naphthoquinone-based (e.g., annulin-B), S-allyl-brassinin, S-benzyl-brassinin, N-[2-(Indol-3-yl)ethyl]-S-methyl-dithiocarbamate, N-[2-(benzo[b]thiophen-3-yl)ethyl]-S-methyl-dithiocarbamate, N-[3-(Indol-3-yl)propyl]-S-methyl-dithiocarbamate, S-hexyl-brassinin, N-[2-(indol-3-yl)ethyl]-S-benzyl-dithiocarbamate, N-[2-(indol-3-yl)ethyl]-S[(naphth-2-yl)methyl]-dithiocarbamate, N-[2-(indol-3-yl)ethyl]-S-[(pyrid-3-yl)methyl]-dithiocarbamate, N-[2-(indol-3-yl)ethyl]-S-[(pyrid-4-yl)methyl]-dithiocarbamate, 5-bromo-brassinin, Phenylimidazole-based IDO inhibitors (e.g., 4-phenylimidazole), Exiguamine A, imidodicarbonimidic diamide,N-methyl-N′-9-phenanthrenyl-monohydrochloride (NSC401366), INCB024360 (epacadostat), 1-cyclohexyl-2-(5H-imidazo[5,1-a]isoindol-5-yl)ethanol (GDC-0919), IDO1-derived peptide, NLG919, Ebselen, Pyridoxal Isonicotinoyl Hydrazone, Norharmane, CAY10581, 2-Benzyl-2-thiopseudourea hydrochloride, and 4-phenylimidazole.
Embodiment 82: The drug delivery vehicle of embodiment 81, wherein said IDO pathway inhibitor comprises 1-methyl-tryptophan.
Embodiment 83: The drug delivery vehicle of embodiment 82, wherein said IDO pathway inhibitor comprises a “D” enantiomer of 1-methyl-tryptophan (indoximod, 1-MT).
Embodiment 84: The drug delivery vehicle of embodiment 82, wherein said IDO pathway inhibitor comprises an “L” enantiomer of 1-methyl-tryptophan (L-MT).
Embodiment 85: The drug delivery vehicle according to any one of embodiments 77-84, wherein said IDO pathway inhibitor, is disposed in a lipid comprising said vesicle and/or conjugated to a lipid comprising said vesicle.
Embodiment 86: The drug delivery vehicle according to any one of embodiments 77-84, wherein said IDO pathway inhibitor, wherein said IDO inhibitor is conjugated to a component of the lipid bilayer.
Embodiment 87: The drug delivery vehicle of embodiment 86, wherein said component of a lipid bilayer comprises a moiety selected from the group consisting of a lipid, PHGP, vitamin E, cholesterol, and a fatty acid.
Embodiment 88: The drug delivery vehicle of embodiment 87, wherein said component of a lipid bilayer comprises cholesterol or a cholesterol derivative.
Embodiment 89: The drug delivery vehicle of embodiment 69, wherein said drug delivery vehicle comprises a hydrophobic therapeutic agent disposed in the lipid bilayer.
Embodiment 90: The drug delivery vehicle of embodiment 89, wherein said hydrophobic therapeutic agent comprises paclitaxel.
Embodiment 91: The drug delivery vehicle according to any one of embodiments 1-90, wherein said drug carrier is conjugated to a moiety selected from the group consisting of a targeting moiety, a fusogenic peptide, and a transport peptide.
Embodiment 92: The drug delivery vehicle of embodiment 91, wherein said drug carrier is conjugated to a peptide that binds a receptor on a cancer cell or tumor blood vessel.
Embodiment 93: The drug delivery vehicle of embodiment 92, wherein said drug carrier is conjugated to an iRGD peptide.
Embodiment 94: The drug delivery vehicle of embodiment 92, wherein said drug carrier is conjugated to a targeting ligand shown in Table 2.
Embodiment 95: The drug delivery vehicle according to any one of embodiments 91-94, wherein said drug carrier is conjugated to transferrin, and/or ApoE, and/or folate.
Embodiment 96: The drug delivery vehicle according to any one of embodiments 91-95, wherein said drug carrier is conjugated to a targeting moiety that comprises an antibody that binds to a cancer marker.
Embodiment 97: The drug delivery vehicle of embodiment 96, wherein said drug carrier is conjugated to a targeting moiety that comprises an antibody that binds a cancer marker shown in Table 1.
Embodiment 98: The drug delivery vehicle according to any one of embodiments 96-97, wherein said antibody is selected from the group consisting of an intact immunoglobulin, an F(ab)′₂, a Fab, a single chain antibody, a diabody, an affibody, a unibody, and a nanobody.
Embodiment 99: The drug delivery vehicle according to any one of embodiments 1-98, wherein said drug carriers in suspension are stable for at least 1 month, or at least 2 months, or at least 3 months, or at least 4 months, or at least 5 months, or at least 6 months when stored at 4° C.
Embodiment 100: The drug delivery vehicle according to any one of embodiments 1-99, wherein said drug delivery vehicle forms a stable suspension on rehydration after lyophilization.
Embodiment 101: The drug delivery vehicle according to any one of embodiments 1-100, wherein said drug delivery vehicles, show reduced drug toxicity as compared to the corresponding free platinum-based drug.
Embodiment 102: The drug delivery vehicle according to any one of embodiments 1-101, wherein said drug delivery vehicle has colloidal stability in physiological fluids with pH 7.4 and remains monodisperse to allow systemic biodistribution and is capable of entering a disease site by vascular leakage (EPR effect) or transcytosis.
Embodiment 103: The drug delivery vehicle according to any one of embodiments 1-102, wherein said carrier is colloidally stable.
Embodiment 104: A pharmaceutical formulation comprising:
   drug delivery vehicle according to any one of embodiments 1-103; and
   a pharmaceutically acceptable carrier.
Embodiment 105: The pharmaceutical formulation of embodiment 104, wherein said formulation is an emulsion, dispersion, or suspension.
Embodiment 106: The pharmaceutical formulation of embodiment 105, wherein said suspension, emulsion, or dispersion is stable for at least 1 month, or at least 2 months, or at least 3 months, or at least 4 months, or at least 5 months, or at least 6 months when stored at 4° C.
Embodiment 107: The pharmaceutical formulation according to any one of embodiments 104-106, wherein the nanovesicle drug carriers, and/or the a drug delivery vehicles, and/or the a nanomaterial carriers in said formulation show a substantially unimodal size distribution; and/or show a PDI less than about 0.2, or less than about 0.1.
Embodiment 108: The pharmaceutical formulation according to any one of embodiments 104-107, wherein said formulation is formulated for administration via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.
Embodiment 109: The pharmaceutical formulation according to any one of embodiments 104-107, wherein said formulation is a sterile injectable.
Embodiment 110: The pharmaceutical formulation according to any one of embodiments 104-109, wherein said formulation is a unit dosage formulation.
Embodiment 111: A method of treating a cancer, said method comprising:
   administering to a subject in need thereof an effective amount of a drug delivery vehicle according to any one of embodiments 1-103; and/or
   a pharmaceutical formulation according to any one of embodiments 104-110.
Embodiment 112: The method of embodiment 111, wherein said method comprises a component of a primary therapy in a chemotherapeutic regimen.
Embodiment 113: The method of embodiment 111, wherein said method comprises an adjunct therapy in a treatment regime that additionally comprises chemotherapy using another chemotherapeutic agent, and/or surgical resection of a tumor mass, and/or radiotherapy.
Embodiment 114: The method according to any one of embodiments 111-113, wherein said composition, a nanovesicle drug carrier, a drug delivery vehicle according, and/or nanomaterial carrier is a component in a multi-drug chemotherapeutic regimen.
Embodiment 115: The method according to any one of embodiments 111-114, wherein said cancer comprises a solid tumor.
Embodiment 116: The method of embodiment 115, wherein said cancer comprises a cancer selected from the group consisting of gastric cancer, hepatocellular carcinoma, head and neck squamous cell carcinoma, urothelial carcinoma, cervical cancer, non-small cell lung cancer, and broadly for non-respectable solid tumors with high microsatellite instability (MSI-H) or DNA mismatch repair deficiency.
Embodiment 117: The method according to any one of embodiments 111-114, wherein said cancer comprises pancreatic cancer.
Embodiment 118: The method according to any one of embodiments 111-114, wherein said cancer comprises colorectal cancer.
Embodiment 119: The method according to any one of embodiments 111-114, wherein said cancer comprises lung cancer.
Embodiment 120: The method according to any one of embodiments 111-114, wherein said cancer is a cancer selected from the group consisting of breast cancer, lung cancer, melanoma, pancreas cancer, liver cancer, acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.
Embodiment 121: The method according to any one of embodiments 1II-120, wherein said administration in conjunction with an additional therapeutic agent.
Embodiment 122: The method of embodiment 121, wherein said drug delivery vehicle is administered as a component FOLFIRINOX protocol that additionally includes folinic acid, 5-fluorouracil, and irinotecan.
Embodiment 123: The method of embodiment 121, wherein said drug delivery vehicle is administered in conjunction with a checkpoint inhibitor.
Embodiment 124: The method of embodiment 123, wherein said checkpoint inhibitor comprises one or more checkpoint inhibitors selected from the group consisting of a PD-L1 inhibitor, a PD-1 inhibitor, and a CTLA-4 inhibitor.
Embodiment 125: The method of embodiment 124, wherein said checkpoint inhibitor comprises one or more PD-L1 inhibitors.
Embodiment 126: The method of embodiment 125, wherein said checkpoint inhibitor comprises an anti-PD-L1 antibody.
Embodiment 127: The method of embodiment 126, wherein said checkpoint inhibitor comprises an anti-PD-L1 antibody selected from the group consisting of Atezolizumab, Avelumab, Durvalumab, BMS-936559, RG-7446. MPDL3280A, MEDI-4736, and MSB0010718C.
Embodiment 128: The method of embodiment 125, wherein said checkpoint inhibitor comprises a peptidic PD-L1 inhibitor.
Embodiment 129: The method of embodiment 128, wherein said PD-L1 inhibitor comprise a moiety selected from the group consisting of AUNP12, CA-170, and BMS-986189.
Embodiment 130: The method according to any one of embodiments 124-129, wherein said checkpoint inhibitor comprises a PD1 inhibitor.
Embodiment 131: The method of embodiment 130, wherein said checkpoint inhibitor comprises an anti-PD1 antibody.
Embodiment 132: The method of embodiment 131, wherein said checkpoint inhibitor comprises an anti-PD1 antibody selected from the group consisting of Nivolumab, Pembrolizumab, Cemiplimab, avelumab, durvalumab, and atezolizumab.
Embodiment 133: The method of embodiment 130, wherein said checkpoint inhibitor comprises an fc fusion with PD-L2.
Embodiment 134: The method of embodiment 133, wherein said checkpoint inhibitor comprises AMP224.
Embodiment 135: The method according to any one of embodiments 124-134, wherein said checkpoint inhibitor comprises CTLA-4 inhibitor.
Embodiment 136: The method of embodiment 135, wherein said CTLA-4 inhibitor comprises Ipilimumab.
Embodiment 137: The method according to any one of embodiments 124-136, wherein said checkpoint inhibitor comprises a bispecific antibody that binds to two checkpoint inhibitors, or an antibody that binds to a checkpoint inhibitor attached to a cytokine.
Embodiment 138: The method of embodiment 137, wherein said checkpoint inhibitor comprises a bispecific antibody that binds to two checkpoint inhibitors.
Embodiment 139: The method of embodiment 138, wherein said bispecific antibody comprises an antibody that binds to PD-1 attached to an antibody that binds to PD-L1, or an antibody that binds to PD-1 attached to an antibody that binds to CTLA4, or an antibody that binds to PD-L1 attached to an antibody that binds to CTLA4.
Embodiment 140: The method of embodiment 139, wherein said bispecific antibody comprises an antibody that binds to PD-1 attached to an antibody that binds to CTLA4.
Embodiment 141: The method of embodiment 137, wherein said checkpoint inhibitor comprises a cytokine attached to an antibody that binds to a checkpoint inhibitor.
Embodiment 142: The method of embodiment 141, wherein said checkpoint inhibitor comprises a cytokine attached to an antibody selected from the group consisting of anti-PD-1, anti-PD-L1, and CTLA4.
Embodiment 143: The method of embodiment 142, wherein said checkpoint inhibitor comprises cytokine attached to an anti-PD-1 antibody.
Embodiment 144: The method of embodiment 143, wherein said checkpoint inhibitor comprises an IL-7 attached to an anti-PD-1 antibody.
Embodiment 145: The method according to any one of embodiments 111-144, wherein said administration is via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.
Embodiment 146: The method according to any one of embodiments 111-144, wherein said administration comprises systemic administration via injection or cannula.
Embodiment 147: The method according to any one of embodiments 111-144, wherein said administration is administration to an intra-tumoral or peri-tumoral site.
Embodiment 148: The method according to any one of embodiments 111-147, wherein said mammal is a human.
Embodiment 149: The method according to any one of embodiments 111-147, wherein said mammal is a non-human mammal.
Embodiment 150: A method of loading silica nanoparticles with a metal-based drug, said method comprising:
   contacting said silica nanoparticles with a cationic form of said metal-based drug at a basic pH to form a mixture of said silica nanoparticles and metal-based drug; and
   applying energy to said mixture where said application of energy enhances loading of said metal-based drug into said silica nanoparticles.
Embodiment 151: The method of embodiment 150, wherein said metal-based drug comprises a metal selected from the group consisting of platinum, palladim, gold, ruthenium, titanium, technetium and rhenium galdolinium, cobalt, lithium, bismuth, iron, calcium, lanthanum, gallium, tin, arsenic, rhodium, copper, zinc, aluminum, lutetium, vanadium, and manganese.
Embodiment 152: The method of embodiment 151, wherein said metal-based drug comprises a metal-based drug selected from the group consisting of a palladium complex drug, a gold complex drug, a ruthenium complex drug, and a titanium complex drug.
Embodiment 153: The method of embodiment 152, wherein said contacting comprises contacting said silica nanoparticles with a cationic activated platinum-based drug at a basic pH to form a mixture of said silica nanoparticles and platinum-based drug.
Embodiment 154: The method of embodiment 153, wherein said activated platinum-based drug comprises a drug selected from the group consisting of 1,2-diaminocyclohexane)platinum(II) (DACHPt), diaminoplatinum(II) (DAPt), and ethylenediamine platinum (EDAPt).
Embodiment 155: The method of embodiment 154, wherein said platinum based chemotherapeutic drug comprises (1,2-diaminocyclohexane)platinum(II) (DACHPt).
Embodiment 156: The method of embodiment 154, wherein said platinum based chemotherapeutic drug comprises diaminoplatinum(II) (DAPt).
Embodiment 157: The method of embodiment 154, wherein said platinum based chemotherapeutic drug comprises ethylenediamine platinum (EDAPt).
Embodiment 158: The method of embodiment 150, wherein said activated platinum-based drug comprises a cationic version of a drug selected from the group consisting of carboplatin, nedaplatin, heptaplatin, lobaplatin, iproplatin, tetraplatin, satraplatin, triplatin tetranitrate, phenanthriplatin, picoplatin, and setraplatin.
Embodiment 159: The method of embodiment 152, wherein said contacting comprises contacting said silica nanoparticles with a cationic version of a drug selected from the group consisting trans-[PdCl₂(2-dqmp)] (2-dqmp=diethyl-2-quinolmethylphosphonate, glycoconjugated Pd(II) complex, [PdCl₂(L)] (L=2-deoxy-2-[(2-pyridinylmethylene) amino]-a-D-glucopyranose, [Au(en)Cl₂][Cl], [Au(dien)Cl][Cl₂], [Au(cyclam)][ClO₄]₂Cl, [Au(terpy)Cl][Cl₂], [Au(phen)Cl₂][Cl], [Au(N—N)Cl₂][PF₆] where N—N is 2,2′-bipyridine, 4,4′-dimethyl-2,2′-bipyridine, 4,4′-dimethoxy-2,2′-bipyridine, or 4,4′-diamino-2,2′-bipyridine, [Au(dach)(pn)]Cl₃ where dach is cis-, or trans-1,2-, or S,S-1,2-diaminocyclohexane and pn is 1,3-diaminopropane, KP1019, NAMI-A, RAPTA-C, RAPTA-T, titanocene X, and titanocene Y.
Embodiment 160: The method according to any one of embodiments 150-159, wherein said pH ranges from about pH 8 up to about pH 9.
Embodiment 161: The method of embodiment 160, wherein said pH is about pH 8.5.
Embodiment 162: The method according to any one of embodiments 150-161, wherein the ratio of drug to silica nanoparticle ranges from about 0.1: about 2 (w/w drug:NP), or from about 0.2:1.5 (w/w drug:NP), or from about 0.2: about 1 (w/w drug: NP).
Embodiment 163: The method of embodiment 162, wherein the ratio of drug to silica nanoparticle is about 0.4:1 (w/w drug:NP).
Embodiment 164: The method according to any one of embodiments 150-163, wherein said method further comprises encapsulating said nanoparticles within lipid bilayers.
Embodiment 165: The method of embodiment 164, wherein said encapsulating comprises using ethanol exchange.
Embodiment 166: The method according to any one of embodiments 150-165, wherein said method produces a nanoparticle drug delivery vehicle according to any one of embodiments 1-103.

### Definitions

The terms “subject,” “individual,” and “patient” may be used interchangeably and refer to humans, as well as non-human mammals (e.g., non-human primates, canines, equines, felines, porcines, bovines, ungulates, rodents, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for a cancer as described herein. Thus, for example, in certain embodiments the subject is a subject with a cancer (e.g., pancreatic ductal adenocarcinoma (PDAC), breast cancer (e.g., drug-resistant breast cancer), colon cancer, brain cancer, and the like). In certain embodiments the methods described herein are prophylactic and the subject is one in whom a cancer is to be inhibited or prevented. In certain embodiments the subject for prophylaxis is one with a family history of cancer and/or a risk factor for a cancer (e.g., a genetic risk factor, an environmental exposure, and the like).

The term “treat” when used with reference to treating, e.g., a pathology or disease refers to the mitigation and/or elimination of one or more symptoms of that pathology or disease, and/or a delay in the progression and/or a reduction in the rate of onset or severity of one or more symptoms of that pathology or disease, and/or the prevention of that pathology or disease. The term “treat” can refer to prophylactic treatment which includes a delay in the onset or the prevention of the onset of a pathology or disease.

The terms “coadministration” or “administration in conjunction with” or “cotreatment” when used in reference to the coadministration of a first compound (or component) (e.g., a Platinum (Pt)-based drug) and a second compound (or component) (e.g., a different cancer therapeutic) indicates that the first compound (or component) and the second compound (or component) are administered so that there is at least some chronological overlap in the biological activity of first compound and the second compound in the organism to which they are administered. Coadministration can include simultaneous administration or sequential administration. In sequential administration there may even be some substantial delay (e.g., minutes or even hours) between administration of the first compound and the second compound as long as their biological activities overlap. In certain embodiments, the coadministration is over a time frame that permits the first compound and second compound to produce an enhanced therapeutic or prophylactic effect on the organism. In certain embodiments the enhanced effect is a synergistic effect.

The terms “nanocarrier”, “nanoparticle drug carrier”, and “drug delivery vehicle” are used interchangeably and refer to a submicron structure (e.g., a nanostructure) having one or a plurality of cavities, e.g., a porous interior. In various embodiments, the cavities contain a cargo that is to be delivered, e.g., to a target cell. In certain embodiments the nanoparticle is a porous silica nanoparticle (e.g., mesoporous silica nanoparticle or “MSNP”). In certain embodiments the nanocarrier comprises a lipid bilayer encasing (or surrounding or enveloping) the core particle.

As used herein, the term “lipid” refers to conventional lipids, phospholipids, cholesterol, chemically functionalized lipids for attachment of PEG, pharmaceutically active ingredients, ligands, etc.

As used herein, the terms “lipid bilayer” or “LB” refers to any double layer of oriented amphipathic lipid molecules in which the hydrocarbon tails face inward to form a continuous non-polar phase.

An activated platinum (activated PT) drug refers to the form of a platinum drug that is pharmaceutically active (e.g., due to the high reactivity of coordinated crosslinking to DNA which stops cancer growth). Platinum drugs exist as an equilibrium of “neutral” or “cationic” species in an aqueous solution. The binding equilibrium is dependent on the Cl— ion concentration (CCl—) as well as pH. While the neutral drug version is dominant in the blood circulation due to a high CCl— concentration (˜150 mM), the formation of an intracellular cationic version is facilitated due to a lower CCl— concentration (˜30 mM). Moreover, the cationic formulation is regarded as pharmaceutically active due to the high reactivity of coordinated crosslinking to DNA, which stops cancer growth.

As used herein, the term “selective targeting” or “specific binding” refers to use of targeting ligands on the surface of a drug delivery nanocarrier (e.g., a LB-coated nanoparticle). In certain embodiments the targeting ligand(s) are on the surface of a lipid bilayer or LB-coated nanoparticle. Typically, the ligands interact specifically/selectively with receptors or other biomolecular components expressed on the target, e.g., a cell surface of interest. The targeting ligands can include such molecules and/or materials as peptides, antibodies, aptamers, targeting peptides, polysaccharides, and the like.

A “silica nanoparticle” refers to a nanoparticle that comprises silica or that consists of silica. In certain embodiments, the silica nanoparticle can include, e but need not be limited to a nanoparticle comprising a functionalized silica.

A coated silica nanoparticle, having targeting ligands can be referred to as a “targeted nanoparticle or a targeted drug delivery nanocarrier, or a targeted silicasome when the nanoparticle is coated with a lipid bilayer.

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. For example, “about” can mean within 1 or more than 1 standard deviation, per the practice in the art. Alternatively, “about” can mean a range of up to 20%, preferably up to 10%, more preferably up to 5% and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “drug” as used herein refers to a chemical entity of varying molecular size, small and large, naturally occurring or synthetic, that exhibits a therapeutic effect in animals and humans. A drug may include, but is not limited to, an organic molecule (e.g., a small organic molecule), a therapeutic protein, peptide, antigen, or other biomolecule, an oligonucleotide, an siRNA, a construct encoding CRISPR cas9 components and, optionally one or more guide RNAs, and the like.

A “pharmaceutically acceptable carrier” as used herein is defined as any of the standard pharmaceutically acceptable carriers. The pharmaceutical compositions of the subject invention can be formulated according to known methods for preparing pharmaceutically useful compositions. The pharmaceutically acceptable carrier can include diluents, adjuvants, and vehicles, as well as carriers, and inert, non-toxic solid or liquid fillers, diluents, or encapsulating material that does not react with the active ingredients of the invention. Examples include, but are not limited to: phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19th ed.) describes formulations that can be used in connection with the drug delivery nanocarrier(s) (e.g., liposomes or nanoparticles encapsulated with a lipid bilayer) described herein.

As used herein, an “antibody” refers to a protein consisting of one or more polypeptides substantially encoded by immunoglobulin genes or fragments of immunoglobulin genes or derived therefrom that is capable of binding (e.g., specifically binding) to a target (e.g., to a target polypeptide). The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Certain preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example Fab′ molecules can be displayed on a phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778). In certain embodiments antibodies should include all that have been displayed on phage (e.g., scFv, Fv, Fab and disulfide linked Fv (see, e.g, Reiter et al. (1995) Protein Eng. 8: 1323-1331) as well as affibodies, unibodies, and the like.

The term “specifically binds”, as used herein, when referring to a biomolecule (e.g., protein, nucleic acid, antibody, etc.), refers to a binding reaction that is determinative of the presence of a biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The term “immunogenic cell death” or “ICD” refers to a unique form of cell death caused by some cytostatic agents such as anthracyclines (Obeid et al. (2007) Nature Med., 13(1): 54-61), anthracenedione (mitoxantrone, aka MTX), oxaliplatin, irinotecan, and bortezomib, or radiotherapy and/or photodynamic therapy (PDT). Unlike regular apoptosis, which is mostly non-immunogenic or even tolerogenic, immunogenic apoptosis of cancer cells can induce an effective antitumor immune response through activation of dendritic cells (DCs) and consequent activation of specific T cell response (Spisek and Dhodapkar (2007) Cell Cycle, 6(16): 1962-1965). Endoplasmic reticulum (ER) stress, reactive oxygen species (ROS) production and induction of autophagy are key intracellular response pathways that govern ICD (Krysko et al. (2012) Nat. Rev. Canc. 12(12): 860-875). In addition to facilitating tumor cell death that facilitates antigen presentation by dendritic cells, ICD is characterized by secretion or release of damage-associated molecular patterns (DAMPs), which exert additional immune adjuvant effects. Calreticulin (CRT), one of the DAMP molecules, which is normally in the lumen of the ER, is translocated to the surface of dying cell where it functions as an “eat me” signal for phagocytes. Other important surface exposed DAMPs are heat-shock proteins (HSPs), namely HSP70 and HSP90, which under stress conditions are also translocated to the plasma membrane. On the cell surface they have an immunostimulatory effect, based on their interaction with number of antigen-presenting cell (APC) surface receptors like CD91 and CD40 and also facilitate cross-presentation of antigens derived from tumor cells on MHC class I molecule, which then triggers CD8⁺ T cell-activation and expansion. Other important DAMPs, characteristic for ICD are secreted, high-mobility group box 1 (HMGB1) protein and ATP (see, e.g., Apetoh et al. (2007) Nature Med. 13(9): 1050-1059; Ghiringhelli et al. (2009) Nature Med. 15(10): 1170-1178). HMGB1 is considered to be a late apoptotic marker and its release to the extracellular space appears to be required for the optimal release and presentation of tumor antigens to dendritic cells. It binds to several pattern recognition receptors (PRRs) such as Toll-like receptor (TLR) 2 and 4, which are expressed on APCs. The most recently found DAMP released during immunogenic cell death is ATP, which functions as a “find-me” signal for monocytes when secreted and induces their attraction to the site of apoptosis (see, e.g., Garg et al. (2012) EMBO J. 31(5): 1062-1079). ATP binds to purinergic receptors on APCs. An inducer of immunogenic cell death is referred to as an ICD inducer.

The terms “IDO inhibitor”, “IDO pathway inhibitor”, and “inhibitor of the IDO pathway) are used interchangeably and refer to an agent (a molecule or a composition) that either partially or fully blocks the activity of indoleamine-2,3-dioxygenase (IDO) and/or partially or fully suppresses the post-enzymatic signaling cascade(s) in the IDO pathway. IDO is an intracellular heme-containing enzyme that initiates the first and rate-limiting step of tryptophan degradation along the kynurenine pathway. The indoleamine 2,3-dioxygenase (IDO) pathway regulates immune response by suppressing cytotoxic T cell function, enhancing regulatory T cell activity (Tregs) and enabling tumor immune escape, either at the tumor or regional lymph node sites. An IDO pathway inhibitor can inhibit the IDO enzyme directly or by interfering or perturbing IDO effector pathway components. Such components include, but are not limited to: IDO2, tryptophan 2,3-dioxygenase (TDO), the mammalian target of rapamycin (mTOR) pathway, aryl hydrocarbon receptor (AhR) pathway, the general control nonderepressible 2 (GCN2) pathway, and the AhR/IL-6 autocrine loop.

### BRIEF DESCRIPTION OF THE DRAWINGS

FIG. 1, panels A-C, illustrates synthesis of activated Pt drugs for the purpose of encapsulation by silicasomes. Panel A) Pt drugs are coordination compounds, with the basic structural composition of cis-[PtA₂X₂]. A₂, i.e., two monodentate or one bidentate ligand with nitrogen donor atoms; X₂: two monodentate or one bidentate anionic ligand(s). Representative Pt drugs studied in this project include oxaliplatin, cisplatin and Pt(en)Cl₂. Panel B) Pt drugs in aqueous solution exist as equilibration species that can be depicted as “neutral” or “cationic”; the cationic variant is pharmaceutically active and capable of providing DNA cross linking. Panel C) Synthesis of cationic and active version of Pt drugs. This resulted in DACHPt (for oxaliplatin), DAPt (for cisplatin) and EDAPt (for Pt(en)Cl₂), respectively. The yield of these reactions is high, i.e. ˜95%.

FIG. 2, panels A-F, illustrates the design, synthesis and characterization of silicasomes, that contain the activated Pt drug. Panel A) The top panel provides a schematic that outlines the synthesis steps for tailored construction of silicasomes, incorporating the active Pt drug. The key parameters that govern successful drug loading are outlined in the table (lower panel). Panel B) pH adjustment to attain weak-basic conditions, favorable for drug loading. At basic pH, silanol groups can be ionized, i.e. ≡Si—OH⇔≡Si—O⁻ (B1), leading to a surface that allows efficient drug attachment via coordination chemistry and electrostatic binding (B2). Panel C) Zeta potential value of bare MSNP is pH-dependent. The development of a negative surface charge at pH 8.5 leads to the highest level of Pt drug binding, as exemplified by DACHPt. Other loading parameters, such the sonication condition, feed ratio, incubation time, etc., were systemically optimized, as illustrated in FIG. 8. Panel D) After experimenting with multiple reaction conditions, it was possible to accomplish a ˜5× increase in terms of EE % and LC %, as determined by ICP-MS analysis of Pt. The same binding level could not be achieved by a passive loading strategy. Panel E) Ultrastructural in situ STEM-EDS imaging confirmed the improved drug loading by coordination chemistry and electrostatic binding. The Pt/Si ratio (w/w) was determined to be 19.4% for the DACHPt silicasome, which is ˜6× higher than a silicasome passively entrapping oxaliplatin. Panel F) An approximate 5-9× fold improvement in drug loading was also achieved for DAPt and EDAPt, according to ICP-MS analysis; this was also confirmed by STEM-EDS visualization. The hydrodynamic sizes of DAPt silicasome and EDAPt silicasome were 138.1±1.5 nm (PDI: 0.113) and 137.8±0.2 nm (PDI: 0.146).

FIG. 3, panels A-E, shows that the DACHPt silicasome improves the PK, biodistribution and anti-cancer efficacy over the free drug in a KPC-derived orthotopic model. The encapsulated delivery also improves drug safety. Panel A) Before animal experimentation, the DACHPt silicasomes were fully characterized, including by cryoEM visualization. The physicochemical properties are summarized. Panels B-C) PK profile in healthy mice (panel B) and Pt drug content in orthotopic KPC tumor (panel C) after the animals received a single IV injection of free oxaliplatin or DACHPt silicasome at identical Pt dose, i.e. 4.95 mg/kg (n=3). PK parameters were calculated by PKSolver software. Pt content was quantified by ICP-MS. Data represent mean±SD; ***, p<0.001 (two-tailed Student's t-test). Panel (D) Comparative efficacy testing of DACHPt silicasome vs free oxaliplatin in the orthotopic KPC model in B6129SF1/J mice. KPC-luc tumor-bearing mice received the free drug or DACHPt silicasomes 8 days after initial tumor implantation. A total of 3 IV administrations were performed. Saline was used as a negative control. In addition to assessing primary tumor size by weight, tumor size and metastases were also assessed by IVIS imaging as shown in the right-hand panel. Data represent mean±SEM; *, p<0.05 (one-way ANOVA followed by a Tukey's test). Panel E) Histological analysis of bone marrow by H&E staining in the efficacy experiment in (panel D). Additional histological analysis to show treatment safety in various organs are shown in FIG. 13.

FIG. 4, panels A-D, shows that DACHPt induces immunogenic cell death (ICD) in the KPC tumor model. Panel A) Schematic to illustrate the action of DACHPt as an inducer of ICD. Select Pt chemo agents, such as DACHPt, induce and immunogenic response in which CRT expression on the dying cancer cell surface provides an “eat-me” signal for APC cells. The response is also accompanied by the release of adjuvant stimuli, such as HMGB1, that promote APC maturation and cross-presentation of endogenous tumor-associated antigens. This can lead to the activation and recruitment of CD8⁺ T cells capable of mediating cytotoxic cancer cell death by the release of perforin. Panel B) Upper panel: Confocal microscopy showing the appearance of CRT on the KPC cell surface treated with oxaliplatin or DACHPt (500 μM) for 24 h. Bar is 20 μm. Green: CRT; Blue: Nuclear. Lower panel: CRT expression was assessed by flow cytometer (left panel) and HMGB1 release was determined by ELSLA (right panel) in KPC cells exposed to oxaliplatin or DACHPt (500 μM) for 24 h. Data are expressed as mean±SD, n=3. *, p<0.05 compared to PBS control (one-way ANOVA followed by a Tukey's test). Panel C) In vivo confirmation of the ICD effect by a vaccination study, in which healthy B6129SF1/J mice first received treatment with the chemo-induced dying KPC cells in one flank on two occasions one week apart, followed by injection of live KPC cells on the contralateral side. Tumors on the contralateral side were collected on day 26. Panel D) The tumor tissue was used for IHC analysis of CD8⁺ and Foxp3⁺ T cell appearance, allowing us to calculate a CD8⁺/Foxp3⁺ ratio. Data are expressed as mean±SEM, n=6. *, p<00.5 (one-way ANOVA followed by a Tukey's test).

FIG. 5, panels A-E, illustrates the immunogenic effects of the DACHPt silicasome in orthotopic PDAC tumors. The data were generated from the same efficacy study described in FIG. 3, panel D. Panels A-C) IHC analysis shows the appearance of ICD biomarkers (CRT and HMGB1) as well as recruitment of activated CD8⁺ and Foxp3⁺ T-cells at the tumor site. A more comprehensive array of panels from the IHC staining data are shown in FIG. 16. Panels D-E) Assessment of perforin (panel D) and granzyme B (panel E) expression in the efficacy study. Data are expressed as mean±SEM, n=3. *, p<0.05; **, p<0.01; ***, p<0.001 (one-way ANOVA followed by a Tukey's test).

FIG. 6 illustrates the results of an animal survival study in the orthotopic KPC model, treated with DACHPt silicasome w/wo anti-PD-1 antibody. KPC tumor-bearing mice were treated with DACHPt silicasome at a Pt dose equivalent of 2 mg/kg IV every 3-4 days, with or without IP administration of 100 μg anti-PD-1 antibody. We also included free oxaliplatin with or without anti-PD-1 for comparison. While chemotherapeutic agent was administrated for 3 times, a total of 4 administrations was given for the antibody, as we outlined on the top panel. Saline and anti-PD-1 antibody alone were also used as controls. Kaplan-Meier plots were used to display the survival rate of the different animal groups (n=5-7, *p<0.05, Log Rank test).

FIG. 7 Common Pt-based antineoplastic molecules that are used in the clinic or in the developmental stage.

FIG. 8, panels A-B, illustrates optimization of Pt drug loading conditions. In addition to the pH effect that was discussed in Example 1, we also compared the method of drug soaking and incubation time on drug loading efficiency (panel A). Moreover, we determined and optimal Pt:MSNP feed ratio (panel B), based on the measurement of loading efficiency and capacity. Preferred conditions are highlighted by arrows.

FIG. 9, panels A-B, illustrates the determination of drug loading % before and after lipid bilayer (LB) coating. Panel A) MSNP was incubated with DACHPt at a weight ratio of 0.2:1 (Pt:MSNP) under weak-basic conditions (pH 8.5). After sonication for 10 min, the particles were used for lipid coating. While the LB coating procedure led to some drug loss, the silicasome (lipid coated MSNP) provided secure cargo trapping, with minimal premature release (see below). Panel B) Moreover, the introduction of a LB on the silica surface dramatically improved the colloidal stability. To illustrate the dispersal characteristics of the suspended particles, photographic images were obtained before and after LB coating. While Pt-laden MSNPs gave a turbid appearance, we discerned an optical transparency in the LB-coated DACHPt laden silicasome. Size, PDI and zeta potential measurements are provided FIG. 3.

FIG. 10 illustrates the drug release profile of the DACHPt-laden silicasome. The drug release of the DACHPt silicasome was tested under abiotic conditions by incubating the particles in H2O, PSF (phagolysosomal simulation fluid, pH 4.5) or 100% FBS (fetal bovine serum) at 37° C., with gentle shaking. Samples obtained at different time points were spun down at 15,000 rpm for 10 min. The Pt content in supernatant was detected by ICP-OES and followed by released drug calculation. Data are expressed as mean±SD. n=3.

FIG. 11, panels A-D, shows cytotoxicity of DACHPt silicasome in different cancer cell lines. The DACHPt silicasome was tested in a list of cancer cell lines to determine the in vitro killing effect, using a MTS assay. These included 2 pancreatic cell lines (panel A, KPC cells and panel B, PANC-1) and 2 colon cancer cells (panel C, MC38 and panel D, CT26). The MTS assay was conducted with free DACHPt and oxaliplatin as controls. Data are expressed as mean±SD. n=3.

FIG. 12 shows the biodistribution of free oxaliplatin and DACHPt laden silicasome in the orthotopic KPC model. Tumor bearing mice received a single IV injection of free oxaliplatin or DACHPt silicasome to deliver the same Pt dose of 4.95 mg/kg. Animals were sacrificed at 48 h. Pt drug content was determined by ICP-MS. Data are expressed as mean±SD. n=3. *, p<0.05 compared to free oxaliplatin (Student's t-test).

FIG. 13 illustrates the results of a safety assessment of the DACHPt silicasome and free drug through histological examination of major organs. H&E staining was performed on main organs collected in the efficacy study described in FIG. 3, panel D. No major histological abnormalities were observed in both treatment groups.

FIG. 14 illustrates use of the MTS assay to find the optimal dose for each ICD stimulus to be used for a vaccination experiment (see FIG. 4, panel C). The MTS assay was performed at 24 h after the treatment with oxaliplatin or DACHPt in KPC cells. Data represents mean±SD, n=3. We decided to use drug dose of 500 μM to ensure ˜70% compromised cell viability in the vaccination experiment.

FIG. 15, panels A-B, shows spaghetti tumor plots to show growth curves (panel A), along with tumor weight determination (panel B) in the vaccination study in FIG. 4, panel C. Data represents mean±SD, n=6. *p<0.05 (1-way ANOVA followed by a Tukey's test). We obtained 1 tumor-free mouse in the treatment using DACHPt.

FIG. 16, panels A-C, shows representative IHC staining to determine (panel A) CRT, (panel B) HMGB1, (panel C) CD8 and FoxP3 expression in tumor tissues collected from the efficacy experiment (FIG. 5). In panel B, the scheme demonstrates the use of Aperio ImageScope software to quantify HMGB1 release in tumor tissues receiving different treatments. High resolution HMGB1 IHC pictures were scanned, followed by a software mediated imaging analysis process, which can discern “pixel density” in the picture. While the strong positive pixel density comes from the nuclear region (non-released HMGB1), the weak- or mid-positive regions come from the released HMGB1. The % of HMGB1 release was calculated by [(weak-positive+mid-positive pixel counts)/(total positive pixel counts)]×100%. Bars represent 50 μm.

### DETAILED DESCRIPTION

As described herein a porous silica nanoparticle (e.g., a mesoporous silica nanoparticle) based platform for the high dose loading and delivery of a range of metal-based therapeutic agents. In certain embodiments, the metal-based therapeutic agents comprise one or more activated platinum chemotherapeutic agents. In various embodiments, illustrative, but non-limiting embodiments, the activated platinum (Pt) chemotherapeutic agents are attached to the silica nanoparticle (e.g., within the pores of a mesoporous silica nanoparticle through the use of electrostatic and coordination chemistry under weak-basic pH conditions). Moreover, in certain embodiments, the nanoparticles are encapsulated in a lipid bilayer thereby forming a “silicasome”. Without being bound to a particular theory, it is believed the presence of the lipid bilayer (LB) improves colloidal stability after intravenous (IV) injection.

The porous silica nanoparticles (e.g., mesoporous silica nanoparticles (MSNPs) have a large interior packaging space for drugs against the walls of the porous interior. This leads to a substantial increase in loading capacity and stable retention until the carrier enters the tumor site to deliver its payload. The presence of a supported lipid bilayer (LB), provides for stable drug encapsulation by an intact surface coat. The LB-coated MSNPs have been labeled “silicasomes” to distinguish them from liposomes, which also contain (a non-supported) LB that encapsulates a fluid space and its content (e.g., a drug).

As described in Example, 1, the PT-loaded silicasomes described herein show improved pharmacokinetics and intratumor delivery of encapsulated oxaliplatin ((1,2-diaminocyclohexane)platinum(II) (DACHPt)), over free drug in an orthotopic Kras-derived pancreatic cancer (PDAC) model. Not only did IV injection of the DACHPt silicasome provide more efficacious cytotoxic tumor cell killing, but could also demonstrate that chemotherapy-induced cell death is accompanied by the features of immunogenic cell death (ICD) as well as a dramatic reduction in bone marrow toxicity. Subsequent performance of a survival experiment demonstrated that the DACHPt silicasome generate a significant improved survival outcome, which could be extended by co-administration of an anti-PD-1 antibody.

In view of the high loading achieved with platinum-based therapeutic agents, it is recognized that the same loading methods can be used with any of a number of other metal-based drugs, e.g., as described herein.

In certain embodiments, the PT-loaded silicasomes described herein can comprise one or more additional therapeutic agents. Such agents can be disposed within the silica nanoparticle or within the lipid bilayer or conjugated to the lipid bilayer. Thus, for example, in certain embodiments the silicasomes described herein can additionally contain one or more inhibitor(s) of the indoleamine 2,3-dioxygenase (IDO) pathway (IDO pathway inhibitor). Without being bound to a particular theory it is believed that such IDO pathway inhibitors can synergize with loaded platinum-based chemotherapeutics.

In certain embodiments, the silicasomes described herein comprise a hydrophobic therapeutic moiety disposed in the lipid bilayer. Thus, for example, in certain embodiments the additional therapeutic moiety can comprise paclitaxel.

Additionally method of making the drug delivery nanoparticles are provided as well as methods of use of the nanoparticles, e.g., in the treatment of a cancer.

### Metal-Based Chemotherapeutic Agents.

The direct loading approach for platinum (Pt)-based drugs typically results in a very low loading capacity. Thus, for example, using the direct drug encapsulation approach, it is only possible to make an oxaliplatin (OX)-laden silicasome with a maximum loading capacity of ˜5% (OX/MSNP w/w) and with a loading efficiency of ˜5% (i.e., 95% of the offered drug was wasted). Without being bound to a particular theory, it is believed that the low loading efficiency/capacity is principally due to the poor water solubility of OX and the lack of specific interaction between OX and the MSNP silica surface.

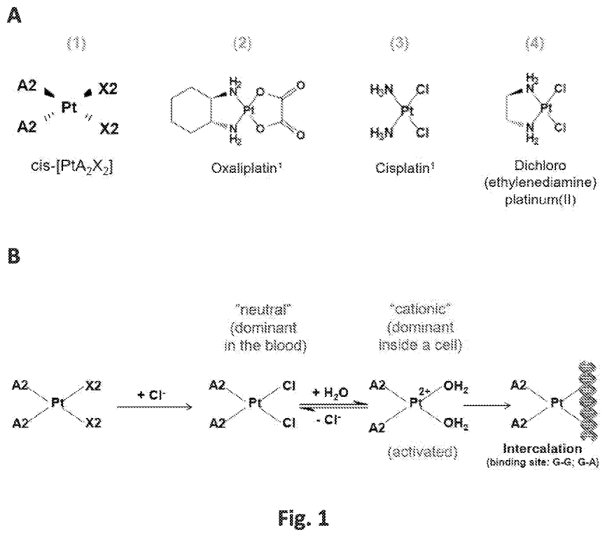

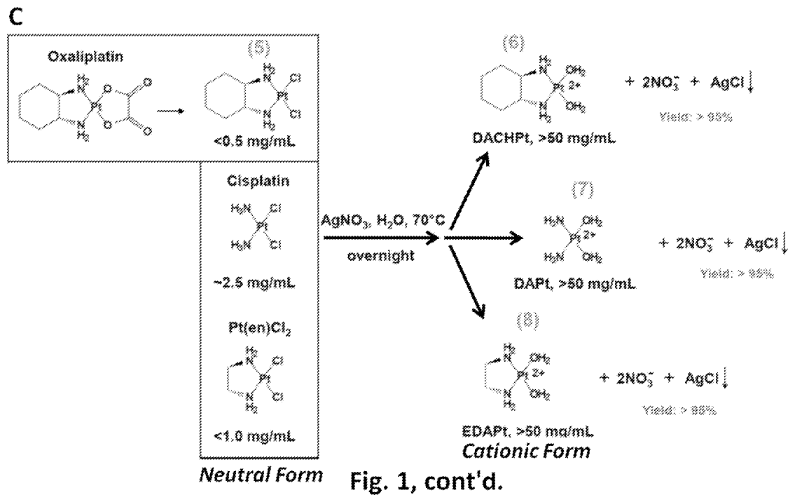

In view of these concerns a novel approach to achieve a high loading capacity for an active version of Pt-based drug (e.g., OX) into the silicasome was developed. Instead of passive encapsulation of the Pt drug, we made use of cationic, activated Pt drugs (e.g., 1,2-cyclohexanediamine platinum (II), a.k.a. DACHPt), for drug loading by interacting with the silanol groups in the walls of the MSNP pores. A conceptualization of the final product is demonstrated in FIG. 1.

The loading of three common Pt drugs is illustrated in Example 1, however, it will be recognized that using these teachings, other Pt-based drugs can readily be loaded. As proof of principle, Example 1 illustrates loading of oxaliplatin, cisplatin and dichloro (ethylenediamine) platinum (Pt(en)Cl₂) (FIG. 1, panel A, structures 2-4)[7b]. It is generally agreed upon that Pt drugs exist as an equilibrium of “neutral” or “cationic” species in an aqueous solution (FIG. 1, panel B)^{[16]}. The binding equilibrium is dependent on the Cl⁻ ion concentration (C_{Cl}_{^{−}}) as well as pH^{[16]}. While the neutral drug version is dominant in the blood circulation due to a high C_{Cl}_{^{−}} concentration (˜150 mM), the formation of an intracellular cationic version is facilitated due to a lower C_{Cl}_{^{−}} concentration (˜30 mM)^{[17]}. Moreover, the cationic formulation is regarded as pharmaceutically active due to the high reactivity of coordinated crosslinking to DNA, which stops cancer growth^{[16]}.

These findings prompted us to consider loading cationic, activated Pt drugs into MSNP rather than working with pristine drugs. This involves the use of “neutral” Pt drugs where the X₂ligand is represented by Cl⁻ ions. Thus, for example, commercially available dichloro(1,2-diaminocyclohexane) platinum(II) (structure 5 in FIG. 1, panel C) was used as the neutral version of oxaliplatin, in addition to the use of cisplatin and Pt(en)Cl₂, which are already in the neutral form. This allowed us to proceed with the synthesis of 3 activated Pt drugs through the introduction of silver nitrate at 70° C., resulting in the formation of DACHPt (structure 6 in FIG. 1, panel C), DAPt (structure 7 in FIG. 1, panel C) and EDAPt (structure 8 in FIG. 1, panel C). These represent the activated versions of oxaliplatin, cisplatin and (Pt(en)Cl₂), respectively, and could be derived from high reaction yields (>95%).

The PT-loaded silicasomes described herein need not be limited to oxaliplatin, cisplatin, and dichloro(ethylenediamine) platinum. Activated cationic versions of numerous other Pt-based therapeutics, can readily be prepared and loaded using the teachings provided herein. Illustrative, but non-limiting additional platinum-based therapeutics include, but are not limited to carboplatin, nedaplatin, heptaplatin, lobaplatin, iproplatin, tetraplatin, satraplatin, triplatin tetranitrate, phenanthriplatin, picoplatin, and setraplatin Illustrative activated cationic versions of these drugs appears in FIG. 1, panel B Cationic forms of carboplatin, nedaplatin, heptaplatin, lobaplatin, iproplatin, tetraplatin, satraplatin, triplatin tetranitrate, phenanthriplatin, picoplatin, and setraplatin are also available.

Additionally, it will be recognized that using the methods described herein, other metal-based drugs can be loaded into the drug delivery vehicles described and provide a high degree of drug loading. In particular, the loading methods are well suited to other metal-based drugs that exhibit similar metal complexation structure. Generally, as long as the metal-based drug can bind to a surface of the nanoparticle through similar electrostatic/coordination interactions it can readily be incorporated into the drug-delivery vehicles described herein.

Numerous metal-based drugs are known and well suited to incorporation into the drug delivery vehicles described herein. For example, such metal-based drugs include, but are not limited to gold-based rugs (e.g., such as auranofin used for rheumatoid arthritis), technetium and rhenium which can be used as radiopharmaceuticals for imaging and radiotherapy, ruthenium which is an anticancer drug. Other possibilities include, but are not limited to metal-based drugs comprising palladium, gadolinium, cobalt, lithium, bismuth, iron, calcium, lanthanum, gallium, tin, arsenic, rhodium, copper, zinc, aluminum, lutetium, vanadium, manganese, and the like (see, e.g., Jurka, et al. (2017) Metal Complexes of Pharmaceutical Substances, Spectroscopic Analyses—Developments and Applications, Eram Sharmin and Fahmina Zafar, IntechOpen, DOI: 10.5772/65390; Sodhi & Paul (2019) Canc. Therapy & Oncol. Int. J. 14(2): 555883. DOI:10.19080/CTOI; and the like).

“Metal containing drugs are important for a few medical applications including diagnosis and treatment. For example, platinum based compounds have been shown to specifically affect head and neck tumors. These coordination complexes are thought to act cross-link DNA in tumor cells. Gold salt complexes have been used to treat Rheumatoid Arthritis. The gold salts are believed to interact with albumin and eventually be taken up by immune cells, triggering anti-mitochondrial effects and eventually cell apoptosis. Lithium (Li₂CO₃) can be used to treat prophylaxis of manic-depression behavior. Zinc can be used topically to heal wounds and Zn+ can be used to treat Herpes and other viruses. Silver has been used to prevent infection at the burn site for burn wound patients. Phosphine ligand compounds containing gold, silver, and/or copper have anti-cancer properties. Lanthanum carbonate often used under the trade name Fosrenol is used as a phosphate binder in patients suffering from chronic kidney disease. Bismuth subsalicylate is used as an antacid. Platinum, Titanium, Vanadium, Iron: cis DDP (cisdiaminedichoroplatinum), titanium, vanadium, and iron have been shown to react with DNA specifically in tumor cells to treat patients with cancer. Barium has been used for X-ray diagnoses, while gadolinium, and manganese are used for magnetic resonance imaging.

Illustrative metal-based drugs that can be incorporated in the nanoparticle drug delivery systems alone or in combination include, but are not limited to the platinum-based drugs described above, as drugs comprising a metal selected from the group consisting of palladim, gold, ruthenium, titanium, technetium and rhenium galdolinium, cobalt, lithium, bismuth, iron, calcium, lanthanum, gallium, tin, arsenic, rhodium, copper, zinc, aluminum, lutetium, vanadium, and manganese. In certain embodiments, the metal-based drug comprises a metal-based drug selected from the group consisting of a palladium complex drug, a gold complex drug, a ruthenium complex drug, and a titanium complex drug.

In certain embodiments, the metal-based drugs include, but are not limited to anti-cancer palladium complexes such as rans-[PdCl₂(2-dqmp)] (2-dqmp=diethyl-2-quinolmethylphosphonate, and glycoconjugated Pd(II) complex, [PdCl₂(L)] (L=2-deoxy-2-[(2-pyridinylmethylene) amino]-a-D-glucopyranose (see, e.g., Table 1, compounds 1-2; Lazarević, et al. (2017) Eur. J. Med. Chem., 142: 8-31; and the like).

In certain embodiments, the metal-based drugs include, but are not limited to anti-cancer gold complexes. These can include for example, a number of Au(III) complexes with multidentate ligands, namely [Au(en)Cl₂][Cl], [Au(dien)Cl][Cl₂], [Au(cyclam)][ClO₄]₂Cl, [Au(terpy)Cl][Cl₂], [Au(phen)Cl₂][Cl], and the like (see, e.g., Table 1, compounds 3-7; Messori, et al. (2000) J. Med. Chem. 43:3541-3548; Eur J Med Chem. (2017) 142:8-31; Lazarević, et al. (2017) Eur. J. Med. Chem., 142: 8-31; and the like).

It is also possible to load Au(III) complexes that contain functionalized bipyridine ligands of the general formula [Au(N—N)Cl₂][PF6] where N—N=2,2′-bipyridine, or 4,4′-dimethyl-2,2′-bipyridine, or 4,4′-dimethoxy-2,2′-bipyridine; or 4,4′-diamino-2,2′-bipyridine) (see, e.g., Table 1, compounds 8-11).

Other possibilities include, but are not limited to Au(III) complexes of the type [Au(dach)(pn)]Cl₃ where dach=cis-; trans-1,2-; and S,S-1,2-diaminocyclohexane and pn=1,3-diaminopropane (see, e.g., Table 1, compounds 12-14).

In certain embodiments, the metal-based drugs include, but are not limited to anti-cancer ruthenium complexes (see, e.g., Table 1, compounds 15-18; Ndagi et al. (2017) Drug Design, Development and Therapy, 11: 599-616; and the like).

In certain embodiments, the metal-based drugs include, but are not limited to anti-cancer titanium complexes, such as titanocenes (see, e.g., Table 1, compounds 19-20; Ndagi et al. (2017) Drug Design, Development and Therapy, 11: 599-616; and the like).

TABLE 1
Illustrative, but non-limiting examples of metal-based therapeutic agents.

| | |
|---|---|
| Metal-based drug | Structure |

Anti-cancer palladium complexes:

| | | |
|---|---|---|
| 1 | trans-[PdCl₂(2-dqmp)] (2- dqmp═diethyl-2- quinolmethylphosphonate | 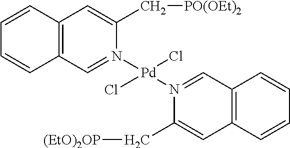 |
| 2 | Glycoconjugated Pd(II) complex, [PdCl₂(L)] (L = 2-deoxy-2-[(2- pyridinylmethylene)amino]-a-D- glucopyranose | 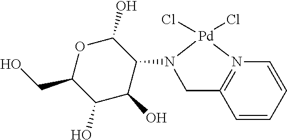 |

Gold complexes:

| | | |
|---|---|---|
| 3 | [Au(en)Cl₂][Cl] | 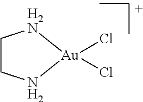 |
| 4 | [Au(dien)Cl][Cl₂] | 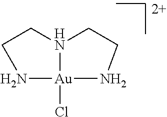 |
| 5 | [Au(cyclam)][ClO₄]₂Cl | 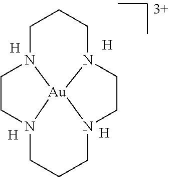 |
| 6 | [Au(terpy)Cl][Cl₂] | 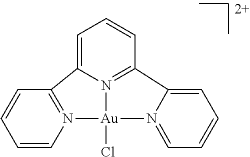 |
| 7 | [Au(phen)Cl₂][Cl] | 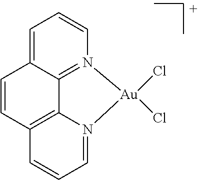 |

Au(III) complex comprising functionalized bipyridine ligands

| | | |
|---|---|---|
| 8 | [Au(N—N)Cl₂][PF6] where N—N═ 2,2′-bipyridine; | 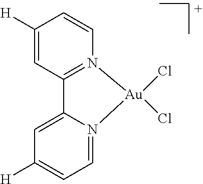 |
| 9 | [Au(N—N)Cl₂][PF6] where N—N═ 4,4′-dimethyl-2,2′-bipyridine | 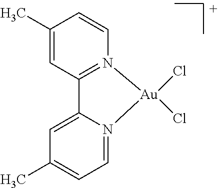 |
| 10 | [Au(N—N)Cl₂][PF6] where N—N═ 4,4′-dimethoxy-2,2′-bipyridine | 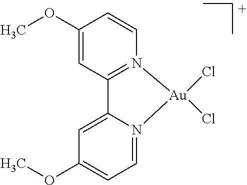 |
| 11 | [Au(N—N)Cl₂][PF6] where N—N═ 4,4′-diamino-2,2′-bipyridine | 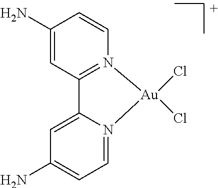 |

Au(III) complexes of the type [Au(dach)(pn)]Cl₃ (where dach = cis-; trans-1,2-; and S,S-
1,2-diaminocyclohexane and pn = 1,3-diaminopropane)

| | | |
|---|---|---|
| 12 | [Au(dach)(pn)]Cl₃ where dach = cis- and pn = 1,3-diaminopropane | 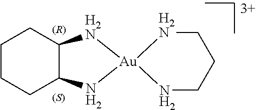 |
| 13 | [Au(dach)(pn)]Cl₃ where dach = trans-1,2- and pn = 1,3- diaminopropane | 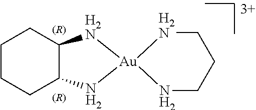 |
| 14 | [Au(dach)(pn)]Cl₃ where dach = diaminocyclohexane and pn = 1,3- diaminopropane | 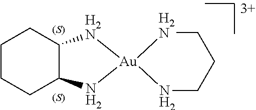 |

Anti-cancer ruthenium complexes

| | | |
|---|---|---|
| 15 | KP1019 | 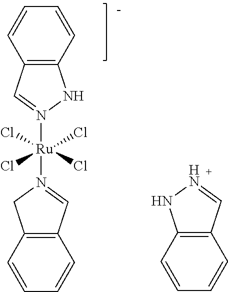 |
| 16 | NAMI-A | 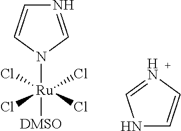 |
| 17 | RAPTA-C | 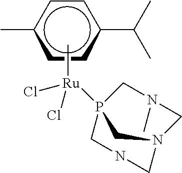 |
| 18 | RAPTA-T | 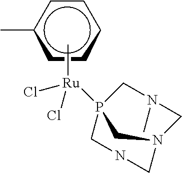 |

Titanocenes:

| | | |
|---|---|---|
| 17 | Titanocene X | 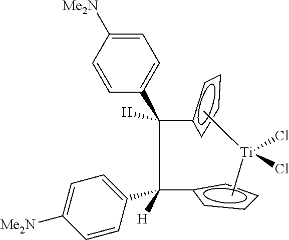 |
| 18 | Titanocene Y | 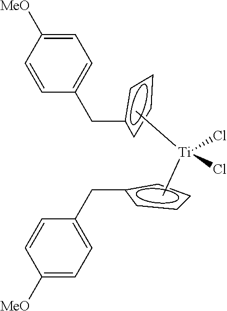 |

The foregoing metal-based drugs are illustrative and non-limiting. Using the teaching provided herein, drug delivery vehicles as described herein carrying numerous other metal-based drugs will be available to one of skill in the art.

### Nanoparticles.

Nanoparticle Fabrication.

In various embodiments the drug delivery vehicles described herein comprise a solid silica nanoparticle or a silica nanoparticle containing one or more cavities where the nanoparticle is disposed within and fully encapsulated by a lipid bilayer.

In certain embodiments the nanoparticle comprise a porous silica nanoparticle. In certain embodiments the porous silica nanoparticle comprises a mesoporous silica nanoparticle (MSN), a mesoporous organosilica nanoparticle (MON), and/or a periodic mesoporous organosilica (PMO) nanoparticle.

MSNs, MONs, and PMOs are commonly fabricated using sol-gel processes in aqueous solutions (Croissant et al. (2015) Nanoscale, 7: 20318-20334; Wu et al. (2013) Chem. Soc. Rev. 42: 3862-3875; Yano & Fukushima (2004) J. Mater. Chem. 14: 1579-1584; Nakamura et al. (2007) J. Phys. Chem. C, 111: 1093-1100). The conventional sol-gel synthesis has been studied extensively and allows precise control of nanoparticle properties such as size, pore size and geometry, particle modification, and/or surface functionalization (see, e.g., Wu et al. (2013) Chem. Soc. Rev. 42: 3862-3875). In one illustrative sol-gel synthesis, silica particles are formed via hydrolysis of various silanes and/or silicates with a subsequent silica condensation:

—Si—O-+HO-Si—→—Si—O—Si-+OH—

In one illustrative, but non-limiting embodiment, synthesis takes place in an aqueous solution and can involve alcohol and ammonia or other catalysts (see, e.g., Yano & Fukushima (2004) J. Mater. Chem. 14: 1579-1584). The speed of the synthesis reaction depends on the pH value with the maximum silica condensation rate at normal pH conditions. The types and concentrations of the synthesis reagents affect the resulting particle size. Tetraethyl orthosilicate (TEOS), tetramethyl orthosilicate (TMOS) and other compounds can be used as silicon sources. To inhibit silica growth and, thus, obtain smaller MSNs, surface-protection agents can be used, such as triethanolamine (TEA), poly (ethylene glycol) (PEG) and/or a second nonionic surfactant (see, e.g., Möller et al. (2007) Adv. Funct. Mater. 17: 605-612). These agents can also be useful for isolation of the growing silica particles from each other, preventing their aggregation and the growth of silica bridges between neighboring particles.

In certain embodiments, to obtain MSNs, micelles can be used as a soft template to form the mesoporous structure. In one illustrative, but non-limiting embodiment, the silica particles are grown on the templates as starting points for the condensation. Surfactants such as cetyltrimethylammonium bromide (CTAB) or cetyltrimethylammonium chloride (CTACl) can be added to the solution as well. At low concentrations just above the critical micellar concentration, the surfactant molecules bind together and form small spherical micelles. At higher concentrations, micelles can have cylindrical or other shapes. These micelles are positively charged and attract negatively charged silanes, facilitating their condensation. Addition of the second surfactant can lead to the formation of the more complicated micellar structures, allowing further modification of the MSNs pore structure. Similar to the micelles, vesicles can be used as templates for the MSN growth (see, e.g., Yeh et al. (2006) Langmuir, 22: 6-9). In certain embodiments, inorganic nanoparticles, such as metal (Au, Pt) or metal oxide (Fe₃O₄) nanoparticles could be incorporated into the structure of MSNs as desired (see, e.g., Kneževi' et al. (2013) RSC Adv. 3: 9584-9593; Timin et al. (2016) Mater. Chem. Phys. 183: 422-429; Ott et al. (2015) Chem. Mater. 2015, 27: 1929-1942). They can be used as the templates for the MSNs growth as well. Such “hybrid” nanoparticles can be capable of both carrying a drug load and acting as contrast agents for bioimaging. In certain embodiments to produce larger pore sizes to accommodate higher quantities of molecules or simply larger molecules (e.g., biomolecules, such as DNA and proteins a swelling agent can be utilized. Several swelling agents can be used to increase the pore sizes, e.g., trimethylbenzene (TMB) (see, e.g., Zhang et al. (2011) J. Colloid Interface Sci. 361: 16-24). Another way to increase the size of the pores is the use of the block-polymers as templates (see, e.g., Han & Ying (2005) Angew. Chem. 117: 292-296).

In one illustrative, but non-limiting synthesis protocol, MSNPs are synthesized by a sol/gel procedure, similar to the method described by Liu et al. (2016) ACS Nano, 10(2): 2702-2715. Thus, for example, to synthesize a batch of ˜100 g MSNP, 17.1 L pure water is added to a 20 L beaker. 0.9 L CTAC solution (25 wt. % in H2O) is gently added while stirring at e.g., 185 rpm, using an overhead shaft for stirring. The solution is heated to 85° C. while stirring and then 72 g triethanolamine in 300 mL H2O is added when the solution reaches a temperature of 85° C. After stirring the solution for another 30 min at 85° C., 600 mL TEOS at 85° C. is gently added, followed by stirring at the same temperature for another ˜4 hr. This yields a milky particle suspension, which is allowed to cool down to room temperature. Six L of ethanol is added to the suspension to precipitate the silica particles, followed by centrifugation at 10,000 rpm for 10 mins. To remove the CTAC, the particles pellets can be resuspended in acidic ethanol (HCl/ethanol, 4:100 v/v) by sonication, followed by repetitive centrifugation (10,000 rpm×60 mins) and resuspension, which is repeated, e.g., 5 times. This is followed by particle washing in pure ethanol, e.g., for 3 times. The purified MSNPs are spun down and resuspended in H2O for the next step of activated PT-drug loading.

The mixture of silane [usually tetraethyl orthosilicate(TEOS)] and an organosilane induces the formation of MONs and PMO. In this case, in certain embodiments, the surfactant templates can be removed with less aggressive extraction procedures, in order not to destroy the inorganic-organic framework of MONs and PMO. In general, harsh pH and temperature conditions are usually employed for the extracting process. The silica-etching chemistry [alkaline or hydrofluoric acid (HF) etching] can be introduced into the synthesis to form the hollow PMO structure (see e.g., Chen et al. (2013) Adv. Mater. 25: 3100-3105). For this, the PMO layer can be directly deposited onto the surface of silica particles in order to form well-defined solid silica core/PMO shell.

The chemical stability of some families of PMOs is higher than for the silica particles under etching. Therefore, the silica core can be selectively removed under alkaline or HF etching conditions, producing hollow periodic mesoporous structure. Illustrative, but non-liming examples of fabrication protocols are described by Wu et al. (2013) Chem. Soc. Rev. 42: 3862-3875 and by Chen et al. (2014) J. Am. Chem. Soc. 136: 16326-16334.

Uniform mesoporous silica particles of different diameters can be prepared using various synthetic conditions (e.g., controlling pH values or time of reaction). For instance, a simple method for tailoring the size of well-ordered and dispersed MSNs by adjusting the pH of the reaction medium, which leads to the series of MSNs with diameter sizes ranging from 30 to 280 nm is described by Lu et al. (2009) Small, 5: 1408-1413. It also possible to control particle growth at different times of the reaction. Smaller particles (140 nm) emerged for 160 s into the reaction process grew to their final size (500 nm) in 600 s.

In one illustrative, but non-limiting embodiment, mesoporous silica nanoparticles (MSNPs) are synthesized as a large batch, as previously described by Liu et al. (2019) ACS Nano. 13(1): 38-53. By way of non-limiting illustration, in certain embodiments, this can involve the addition of 0.9 L of 25 wt % CTAC in water to 17.1 L pure water in a beaker, stirred at 85° C. 72 g triethanolamine is added, followed by 600 mL TEOS. After stirring for 4 hours and cooling to room temperature, the bare MSNPs are precipitated with ethanol and CTAC is removed by washing in acidic ethanol, with sonication. MSNPs at 80 mg/mL in ethanol are centrifuged at 21,000×g for 15 minutes to pellet the nanoparticles. After removal of the ethanol supernatant, the MSNP pellet is resuspended in 123 mM ammonium sulfate in water by bath sonication.

Potential bioaccumulation is one of the biggest limitations for silica nanodrug delivery systems in cancer. Accordingly, in certain embodiments, the porous silica nanoparticles described herein (e.g., mesoporous silica nanoparticles) are modified to improve degradation and clearance. In one illustrative, but non-limiting example, the nanoparticles comprise a mesoporous silica/hydroxyapatite (MSNs/HAP) hybrid drug carrier, that provides enhanced biodegradability of silica. Synthesis of such nanoparticles is described by Hao et al. (2015) ACS Nano, 9(10): 9614-9625.

Other approaches for improving silica nanoparticle degradation include, but are not limited to noncovalent organic doping of silica, covalent incorporation of either hydrolytically stable or redox- and enzymatically cleavable silsesquioxanes, as well as bridged silsesquioxane (BS), and periodic mesoporous organosilica (PMO) NPs. Inorganically doped silica particles such as calcium-, iron-, manganese-, and zirconium-doped NPs, can also be used (see, e.g., Croissant et al. (2017) Adv. Mater., 29: 1604634).

In certain embodiments the mesoporous silica nanoparticles can be imine-doped silica nanoparticles. These nanoparticles contain imine groups embedded within the silica framework (see, e.g., Travaglini et al. (2019) Mater. Chem. Front., 3: 111-119). These methods of increasing degradability of silica nanoparticles are illustrative and non-limiting. Using the teaching provided herein, numerous other porous silica nanoparticles modified for enhanced biodegradation will be available to one of skill in the art.

Illustrative mesoporous silica nanoparticles include, but are not limited to MCM-41, MCM-48, and SBA-15 (see, e.g., Katiyar et al. (2006) J. Chromatog. 1122(1-2): 13-20).

In various embodiments the nanoparticles comprising the drug delivery vehicles described herein (e.g., “core” silica nanoparticles) can include particles as large (e.g., average or median diameter (or other characteristic dimension) as about 1000 nm. However, in various embodiments the nanoparticles are typically less than 500 nm or less than about 300 nm as, in general, particles larger than 300 nm may be less effective in entering living cells or blood vessel fenestrations. In certain embodiments the nanoparticles range in size from about 40 nm, or from about 50 nm, or from about 60 nm up to about 100 nm, or up to about 90 nm, or up to about 80 nm, or up to about 70 nm. In certain embodiments the nanoparticles range in size from about 60 nm to about 70 nm. Some embodiments include nanoparticles having an average maximum dimension between about 50 nm and about 1000 nm. Other embodiments include nanoparticles having an average maximum dimension between about 50 nm and about 500 nm. Other embodiments include nanoparticles having an average maximum dimension between about 50 nm and about 200 nm. In some embodiments, the average maximum dimension is greater than about 20 nm, greater than about 30 nm, greater than 40 nm, or greater than about 50 nm. Other embodiments include nanoparticles having an average maximum dimension less than about 500 nm, less than about 300 nm, less than about 200 nm, less than about 100 nm or less than about 75 nm. As used herein, the size of the nanoparticle refers to the average or median size of the primary particles, as measured by transmission electron microscopy (TEM) or similar visualization technique.

In certain embodiments the drug delivery vehicles (including lipid bilayer) have an average hydrodynamic diameter ranging from about 30 nm up to about 300 nm, or from about 40 nm up to about 200 nm, or from about 50 up to about 100 nm, or from about 60 nm up to about 90 nm, or from about 70 nm up to about 90 nm, or from about 80 nm up to about 90 nm by DLS. In certain embodiments, the drug delivery vehicles have an average hydrodynamic diameter ranging from about 79 nm up to about 86 nm by DLS. In certain embodiments, the drug delivery vehicles have an average diameter ranging from about 30 nm up to about 300 nm, or from about 50 nm up to about 250 nm, or from about 70 nm up to about 200 nm, or from about 90 nm up to about 150 nm, or from about 110 nm up to about 150 nm by cryoEM. In certain embodiments, the vehicles have an average diameter ranging from about 136 nm up to about 139 nm by cryoEM.

Illustrative mesoporous silica nanoparticles include, but are not limited to MCM-41, MCM-48, and SBA-15 (see, e.g., Katiyar et al. (2006) J. Chromatog. 1122(1-2): 13-20).

Using the teachings provided herein, silica nanoparticles are readily available to those of skill in the art and, using the teaching described herein, can be used in the fabrication of the drug delivery vehicles described herein.

Pt-Drug Loading of Nanoparticle.

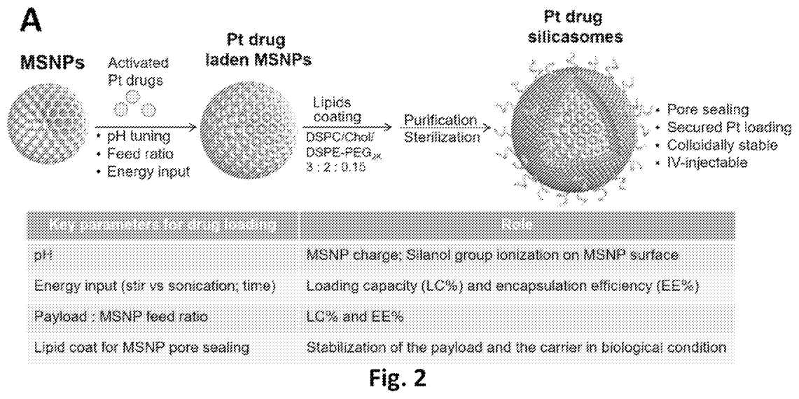

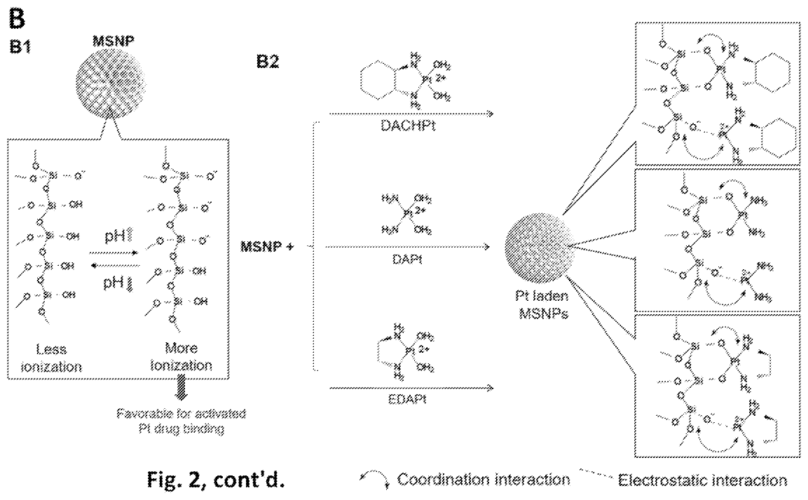

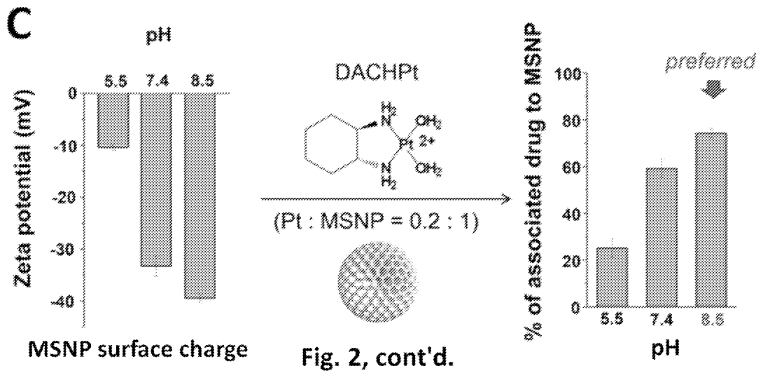

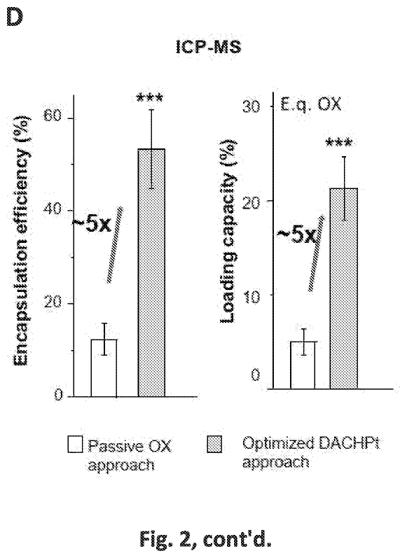

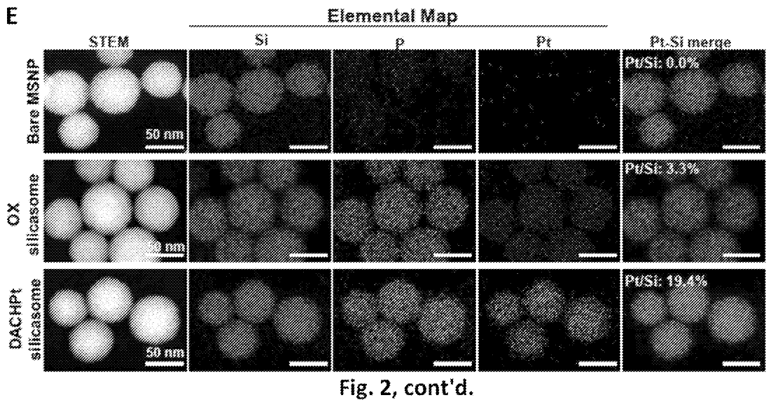

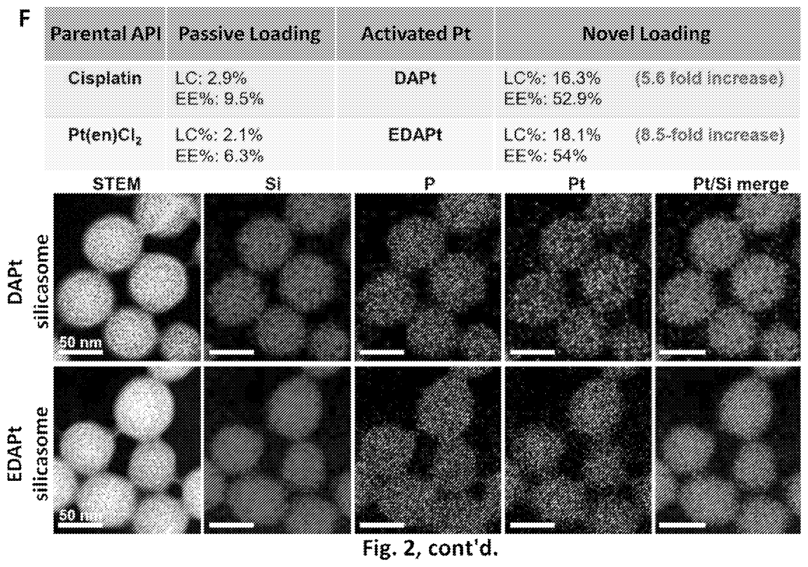

In various embodiments the silica nanoparticles are loaded with platinum-based drugs using a combination of coordination and electrostatic interactions. Since the silanol group density on the silica nanoparticle surface(s) (e.g., pore surfaces) an important role in the surface binding of the activated platinum compound (e.g., DACHPt) (see FIG. 2), it is desirable to control the pH during drug loading. Thus, the loading efficiency and capacity was optimized through control of. pH, incubation time, the soaking process, and variation of the drug/particle ratio, etc. Thus, for example, 20 mg MSNP resuspend in 1 mL HEPES buffer (0.1 M, pH 8.5) was mixed with DACHPt complex (4 mg Pt, equal to 8 mg OX). The mixture was sonicated in water bath sonication for 10 mins and then spun down at 15 K rpm for 10 mins. Non-bound Pt in the supernatant, as detected by ICP-OES was used to determine the loading capacity [%=(total Pt—Pt in supernatant)/amount of MSNP*100} and loading efficiency [%=(total Pt—Pt in supernatant)/total Pt*100].

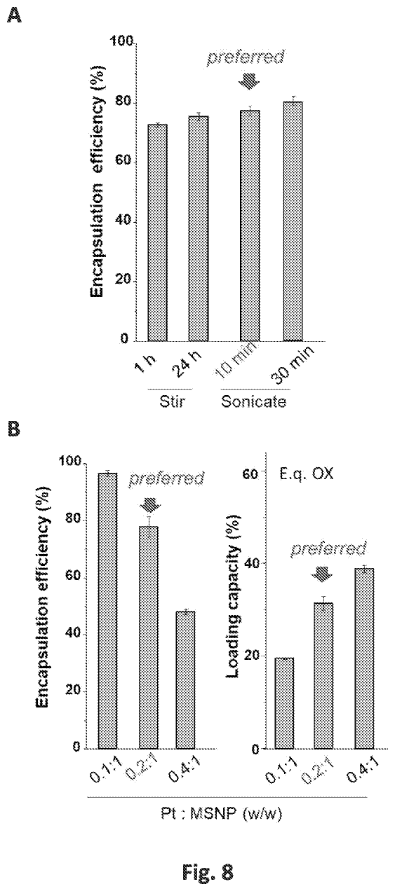

Various key parameters that govern successful drug loading are outlined in FIGS. 2 and 8. At basic pH, silanol groups can be ionized, i.e. ≡Si—OH⇔≡Si—O⁻ (B1), leading to a surface that allows efficient drug attachment via coordination chemistry and electrostatic binding (FIG. 2, panel B, B2). Panel C) Zeta potential value of bare MSNP is pH-dependent. The development of a negative surface charge at pH 8.5 leads to the highest level of Pt drug binding, as exemplified by DACHPt. Other loading parameters, such the sonication condition, feed ratio, incubation time, etc., were systemically optimized, as illustrated in FIG. 8. After experimenting with multiple reaction conditions, it was possible to accomplish a ˜5× increase in terms of EE % and LC %, as determined by ICP-MS analysis of Pt. The same binding level could not be achieved by a passive loading strategy. Ultrastructural in situ STEM-EDS imaging confirmed the improved drug loading by coordination chemistry and electrostatic binding. The Pt/Si ratio (w/w) was determined to be 19.4% for the DACHPt silicasome, which is ˜6× higher than a silicasome passively entrapping oxaliplatin. Additionally, an approximate 5-9× fold improvement in drug loading was also achieved for DAPt and EDAPt, according to ICP-MS analysis; this was also confirmed by STEM-EDS visualization.

This optimization of PT-based drug loading is illustrative and non-limiting. Using the teaching provided herein a combination of electrostatic and coordination interactions can be provided for loading of essentially any cationic, activated platinum drug.

Lipid bilayer (LB)

Bilayer composition.

The drug carrier nanoparticles described herein comprise a silica nanoparticle comprising one or more cavities, e.g., a porous nanoparticle such as a mesoporous silica nanoparticle (MSNP)), coated with a lipid bilayer. In certain embodiments the bilayer composition is optimized to provide a rapid and uniform particle coating, to provide colloidal and circulatory stability, and to provide effective cargo retention, while also permitting a desirable cargo release profile.

In certain embodiments the lipid bilayer comprises a combination of a phospholipid, and cholesterol, and in certain embodiments, a pegylated lipid (e.g., PE-PEG₂₀₀₀, DSPE-PEG₂₀₀₀), or a factionalized pegylated lipid (e.g., DSPE-PEG₂₀₀₀-maleimide) to facilitate conjugation with targeting moieties or other moieties including, for example, a drug.

In certain illustrative, but non-limiting embodiments the lipid bilayer can comprise: 1) one or more saturated fatty acids with C14-C20 carbon chain, such as phosphatidylethanolamine (PE), dimyristoylphosphatidylcholine (DMPC), dipalmitoylphosphatidylcholine (DPPC), distearoylphosphatidylcholine (DSPC), and diactylphosphatidylcholine (DAPC); and/or 2) One or more unsaturated fatty acids with a C14-C20 carbon chain, such as 1,2-dimyristoleoyl-sn-glycero-3-phosphocholine, 1,2-dipalmitoleoyl-sn-glycero-3-phosphocholine,1,2-dioleoyl-sn-glycero-3-phosphocholine (DOPC), 1,2-dieicosenoyl-sn-glycero-3-phosphocholine; and/or 3) natural lipids comprising a mixture of fatty acids with C12-C20 carbon chain, such as Egg PC, and Soy PC, sphingomyelin, and 4) cholesterol (CHOL) and/or a modified cholesterol (e.g., cholesterol hemisuccinate (CHEMS)) the like. It is noted that, in certain embodiments, in order to compensate a positive charge, it is possible to use cholesteryl hemisuccinate (CHEMS) that carries one negative charge at pH >6.5 in the formulation. These lipids are illustrative but non-limiting and numerous other lipids are known and can be incorporated into a lipid bilayer for formation of a drug delivery nanocarrier (e.g., a bilayer-coated nanoparticle).

In certain embodiments the drug carrier comprises bilayer comprising a lipid (e.g., a phospholipid), cholesterol, and a PEG functionalized lipid (e.g., a mPEG phospholipid). In certain embodiments the mPEG phospholipids comprises a C14-C18 phospholipid carbon chain from, and a PEG molecular weight from 350-5000 (e.g., MPEG 5000, MPEG 3000, MPEG 2000, MPEG 1000, MPEG 750, MPEG 550, MPEG 350, and the like). In certain embodiments the mPEG phospholipid comprises DSPE-PEG5000, DSPE-PEG3000, DSPE-PEG2000, DSPE-PEG1000, DSPE-PEG750, DSPE-PEG550, or DSPE-PEG350, PE-PEG5000, PE-PEG3000, PE-PEG2000, PE-PEG1000, PE-PEG750, PE-PEG550, PE-PEG350, and the like. MPEGs are commercially available (see, e.g.,//avantilipids.com/product-category/products/polymers-polymerizable-lipids/mpeg-phospholipids).

In certain embodiments lipid bilayer comprises an mPEG phospholipid with a phospholipid C14-C18 carbon chain, and a PEG. In certain embodiments, the PEG molecular weight ranges from about 350 Da to about 5000 Da. In certain embodiments the lipid bilayer comprises PE-PEG_{2K}. In certain embodiments the lipid bilayer comprises PE-PEG_{5K}.

In certain embodiments, the said lipid bilayer comprises DPSC, cholesterol, and PE-PEG. In certain embodiments, the ratio of DPSC:cholesterol:PE-PEG ranges from 40-90% DSPC:10%-50% Chol:1%-10% PE-PEG (molar ratio). In certain embodiments, the bilayer comprises DSPC:cholesterol:PE-PEG at a molar ratio of about 3:2:0.15 for DSPC, cholesterol, and PE-PEG, respectively.

In certain embodiments, the lipid bilayer comprises a cholesterol derivative selected from the group consisting of cholesterol hemisuccinate (CHEMS), lysine-based cholesterol (CHLYS), and PEGylated cholesterol (Chol-PEG). In certain embodiments, the lipid bilayer comprises CHEMS. In certain embodiments, the bilayer comprises CHEMS ranging from about 5% (mol percent) up to about 30% total lipid. In certain embodiments, the bilayer comprises about 10% or about 20% CHEMS or about 30% CHEMS or about 40% CHEMS. In certain embodiments, the cholesterol derivative is used in place of said cholesterol.

The foregoing lipid bilayer compositions are illustrative, but non-limiting. Using the teachings provided herein numerous other lipid bilayer compositions will be available to one of skill for incorporation into the silicasomes described herein.

Encapsulation of Silica Nanoparticle by Lipid Bilayer.

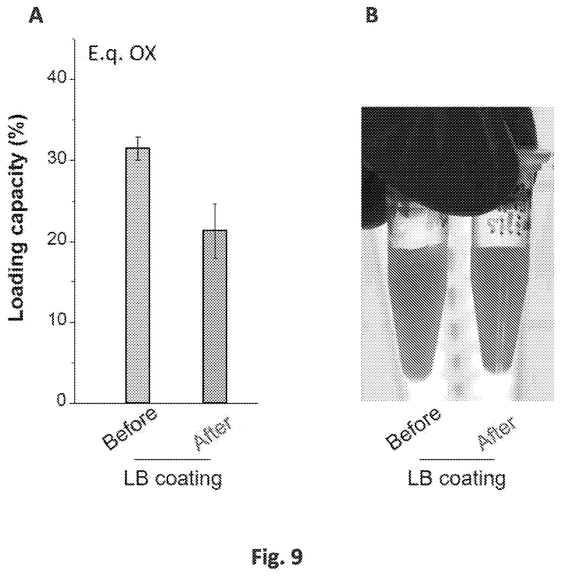

In various embodiments, the silica nanoparticles are coated (encapsulated) with a lipid bilayer by an ethanol exchange method that results in the formation of the bilayer encapsulated nanoparticle. The ethanol exchange s bilayer method provides rapid and uniform pore sealing, capable of entrapping drug payloads of ˜70% into the porous interior (see, e.g., FIG. 9). Notably, in various embodiments methods of applying the lipid bilayer expressly exclude the use of already formed liposomes (a.k.a., liposome fusion), but rather utilize a mixture of lipids and/or a lipid film that is not a component of a liposome.

In one illustrative, but non-limiting embodiment, following the synthesis of the drug-soaked (e.g., DACHPt soaked) in bare particles, the MSNPs are subsequently coated by a lipid bilayer (LB) as follows: Briefly, a mixture of lipids (e.g., 16 mg DSPC, 5.4 mg, cholesterol (Chol) and 2.8 mg DSPE-PEG2000), yielding a DSPC/Chol/DSPE-PEG2000 molar ratio of 3:2:0.15) is dissolved in 50 μL pure ethanol at ˜65° C. The drug-laden MSNPs (e.g., DACH-Pt laden MSNPs), are resuspended in 500 μL preheated (˜65° C.). Dextrose/HEPES buffer (e.g., 5% dextrose, 5 mM HEPES, pH7.4), is added to the lipid solution by pipette mixing. The mixture is treated by probe sonication (e.g., power=52 W) using, e.g., a 15 s/5 s on/off cycle for ˜10 min. The coated DACHPt silicasomes are washed (e.g., 3 times using a HEPES-buffered dextrose solution (5% dextrose, 5 mM HEPES, pH7.4)). The sample is processed by filtration using a 0.2 μm filter for sterilization. Using this method, or minor variations thereof, lipid bilayers of numerous different formulations can readily be formed on drug-containing silica nanoparticles.

In certain embodiments, to attach a surface LB coating, a coated lipid film procedure can be utilized in which nanoparticle (e.g., MSNP) suspensions are added to a large lipid film surface, coated on, e.g., a round-bottom flask. Using different lipid bilayer compositions, a series of experiments can be performed to find a composition and optimal lipid/particle ratio that provides rapid and uniform particle wrapping, coating and effective cargo retention and/or release upon sonication. It is believed that this lipid composition and wrapping cannot be achieved by liposomal fusion to the particle surface under low energy vortexing conditions.

### Combined Drug Delivery

In certain embodiments the drug delivery vehicles described herein can contain an additional cargo (in addition to a platinum-based drug, or other metal-based drug as described above) on the surface and/or in the cavities of the nanoparticle (when such cavities are present). In certain embodiments, the additional cargoes comprise an additional metal-based drug, as described above. In certain embodiments such additional cargoes comprise one or more cancer therapeutic agents. In certain embodiments the additional agents are cancer therapeutic agents capable of being loaded, e.g., according to the methods described herein. In certain embodiments, the additional agents comprise anti-cancer therapeutic agents that can be functionalized to be capable of being loaded, e.g., according to the methods described herein.

Additional illustrative, but non-limiting additional therapeutic agents include, but are not limited to alkaloids (e.g. irinotecan, topotecan, 10-hydroxycamptothecin, belotecan, rubitecan, vinorelbine, LAQ824, vinblastine, vincristine, homoharringtonine, trabectedin), anthracyclines (e.g. doxorubicin, epirubicin, pirarubicin, daunorubicin, rubidomycin, valrubicin, amrubicin), alkaline anthracenediones (e.g. mitoxantrone), alkaline alkylating agents (e.g. cyclophosphamide, mechlorethamine, temozolomide), purine or pyrimidine derivatives (e.g. 5-fluorouracil, 5′-deoxy-5-fluorouridine, gemcitabine, capecitabine) and protein kinase inhibitors (e.g., pazopanib, enzastaurin, vandetanib erlotinib, dasatinib, nilotinib, sunitinib, osimertinib, palbociclib, ribociclib), and the like.

In certain embodiments, embodiments the additional therapeutic agent comprise an inhibitor of the IDO pathway. Without being bound by a particular theory, it is believed that an IDO inhibitor will synergize with an inducer of cell death such as indoximod and the like (see, e.g., PCT Patent Application No: PCT/US2018/033265. In certain embodiments, the IDO pathway inhibitor comprises an agent selected from the group consisting of of D-1-methyl-tryptophan (indoximod, D-1MT), L-1-methyl-tryptophan (L-1MT), a mixture of D-1MT and L-1MT, 1-methyl-L-tryptophan (L-1MT), methylthiohydantoin-dl-tryptophan (MTH-Trp, Necrostatin), β-carbolines (e.g., 3-butyl-p-carboline), Naphthoquinone-based (e.g., annulin-B), S-allyl-brassinin, S-benzyl-brassinin, N-[2-(Indol-3-yl)ethyl]-S-methyl-dithiocarbamate, N-[2-(benzo[b]thiophen-3-yl)ethyl]-S-methyl-dithiocarbamate, N-[3-(Indol-3-yl)propyl]-S-methyl-dithiocarbamate, S-hexyl-brassinin, N-[2-(indol-3-yl)ethyl]-S-benzyl-dithiocarbamate, N-[2-(indol-3-yl)ethyl]-S[(naphth-2-yl)methyl]-dithiocarbamate, N-[2-(indol-3-yl)ethyl]-S-[(pyrid-3-yl)methyl]-dithiocarbamate, N-[2-(indol-3-yl)ethyl]-S-[(pyrid-4-yl)methyl]-dithiocarbamate, 5-bromo-brassinin, Phenylimidazole-based IDO inhibitors (e.g., 4-phenylimidazole), Exiguamine A, imidodicarbonimidic diamide,N-methyl-N′-9-phenanthrenyl-monohydrochloride (NSC401366), INCB024360 (epacadostat), 1-cyclohexyl-2-(5H-imidazo[5,1-a]isoindol-5-yl)ethanol (GDC-0919), IDO1-derived peptide, NLG919, Ebselen, Pyridoxal Isonicotinoyl Hydrazone, Norharmane, CAY10581, 2-Benzyl-2-thiopseudourea hydrochloride, and 4-phenylimidazole. In certain embodiments, the IDO pathway inhibitor comprises 1-methyl-tryptophan. In certain embodiments, the IDO pathway inhibitor comprises a “D” enantiomer of 1-methyl-tryptophan (indoximod, 1-MT). In certain embodiments, the IDO pathway inhibitor comprises an “L” enantiomer of 1-methyl-tryptophan (L-MT).

In certain embodiments, the IDO pathway inhibitor, is disposed in a lipid comprising said vesicle and/or conjugated to a lipid comprising said vesicle. In certain embodiments, the IDO inhibitor is conjugated to a component of the lipid bilayer (e.g., lipid, PHGP, vitamin E, cholesterol, a fatty acid, etc.). In certain embodiments, the IDO inhibitor is conjugated to cholesterol. In certain embodiments, the IDO inhibitor is conjugated to a cholesterol derivative.

Alternatively, or additionally, in certain embodiments, hydrophobic compounds can be incorporated into the lipid bilayer surrounding the nanoparticle. Thus, for example, paclitaxel can be incorporated in the lipid bilayer.

The foregoing compounds are illustrative and non-limiting. Using the teachings provided herein, numerous other additional cargoes can be incorporated in the drug delivery vehicles described herein.

### Targeting Ligands.

In certain embodiments the drug delivery vehicles described herein can be conjugated to one or more targeting ligands, e.g., to facilitate specific delivery in endothelial cells, to cancer cells, to fusogenic ligands, e.g., to facilitate endosomal escape, ligands to promote transport across the blood-brain barrier, and the like.

In one illustrative, but non-limiting embodiment, the delivery vehicles described herein is conjugated to a fusogenic peptide such as histidine-rich H5WYG (H₂N-GLFHAIAHFIHGGWHGLIHGWYG-COOH, (SEQ ID NO:1)) (see, e.g., Midoux et al., (1998) Bioconjug. Chem. 9: 260-267).

In certain embodiments delivery vehicles described herein are conjugated to one or more targeting ligand(s) that can include antibodies as well as targeting peptides. Targeting antibodies include, but are not limited to intact immunoglobulins, immunoglobulin fragments (e.g., F(ab)′2, Fab, etc.) single chain antibodies, diabodies, affibodies, unibodies, nanobodies, and the like. In certain embodiments antibodies will be used that specifically bind a cancer marker (e.g., a tumor associated antigen). A wide variety of cancer markers are known to those of skill in the art. The markers need not be unique to cancer cells, but can also be effective where the expression of the marker is elevated in a cancer cell (as compared to normal healthy cells) or where the marker is not present at comparable levels in surrounding tissues (especially where the chimeric moiety is delivered locally).

Illustrative cancer markers include, for example, the tumor marker recognized by the ND4 monoclonal antibody. This marker is found on poorly differentiated colorectal cancer, as well as gastrointestinal neuroendocrine tumors (see, e.g., Tobi et al. (1998) Cancer Detection and Prevention, 22(2): 147-152). Other important targets for cancer immunotherapy are membrane bound complement regulatory glycoproteins CD46, CD55 and CD59, which have been found to be expressed on most tumor cells in vivo and in vitro. Human mucins (e.g. MUC1) are known tumor markers as are gp100, tyrosinase, and MAGE, which are found in melanoma. Wild-type Wilms' tumor gene WT1 is expressed at high levels not only in most of acute myelocytic, acute lymphocytic, and chronic myelocytic leukemia, but also in various types of solid tumors including lung cancer.

Acute lymphocytic leukemia has been characterized by the TAAs HLA-Dr, CD1, CD2, CD5, CD7, CD19, and CD20. Acute myelogenous leukemia has been characterized by the TAAs HLA-Dr, CD7, CD13, CD14, CD15, CD33, and CD34. Breast cancer has been characterized by the markers EGFR, HER2, MUC1, Tag-72. Various carcinomas have been characterized by the markers MUC1, TAG-72, and CEA. Chronic lymphocytic leukemia has been characterized by the markers CD3, CD19, CD20, CD21, CD25, and HLA-DR. Hairy cell leukemia has been characterized by the markers CD19, CD20, CD21, CD25. Hodgkin's disease has been characterized by the Leu-M1 marker. Various melanomas have been characterized by the HMB 45 marker. Non-Hodgkins lymphomas have been characterized by the CD20, CD19, and Ia marker. And various prostate cancers have been characterized by the PSMA and SE10 markers.

In addition, many kinds of tumor cells display unusual antigens that are either inappropriate for the cell type and/or its environment, or are only normally present during the organisms' development (e.g., fetal antigens). Examples of such antigens include the glycosphingolipid GD2, a disialoganglioside that is normally only expressed at a significant level on the outer surface membranes of neuronal cells, where its exposure to the immune system is limited by the blood-brain barrier. GD2 is expressed on the surfaces of a wide range of tumor cells including neuroblastoma, medulloblastomas, astrocytomas, melanomas, small-cell lung cancer, osteosarcomas and other soft tissue sarcomas. GD2 is thus a convenient tumor-specific target for immunotherapies.

Other kinds of tumor cells display cell surface receptors that are rare or absent on the surfaces of healthy cells, and which are responsible for activating cellular signaling pathways that cause the unregulated growth and division of the tumor cell. Examples include (ErbB2) HER2/neu, a constitutively active cell surface receptor that is produced at abnormally high levels on the surface of breast cancer tumor cells.

Other useful targets include, but are not limited to CD20, CD52, CD33, epidermal growth factor receptor and the like.

An illustrative, but not limiting list of suitable tumor markers is provided in Table 2. Antibodies to these and other cancer markers are known to those of skill in the art and can be obtained commercially or readily produced, e.g. using phage-display technology. Such antibodies can readily be conjugated to the drug delivery vehicles (e.g., LB-coated nanoparticle) described herein, e.g., in the same manner that iRGD peptide is conjugated in Example 3.

TABLE 2
Illustrative cancer markers and associated references, all of which are incorporated
herein by reference for the purpose of identifying the referenced tumor markers.

| Marker | Reference |
|---|---|
| 5 alpha reductase | Délos et al. (1998) Int J Cancer, 75:6 840-846 |
| α-fetoprotein | Esteban et al. (1996) Tumour Biol., 17(5): 299-305 |
| AM-1 | Harada et al. (1996) Tohoku J Exp Med., 180(3): 273-288 |
| APC | Dihlmannet al. (1997) Oncol Res., 9(3) 119-127 |
| APRIL | Sordat et al. (\grave{ }998) J Exp Med., 188(6): 1185-1190 |
| BAGE | Böel et al. (1995) Immunity, 2: 167-175. |
| β-catenin | Hugh et al. (1999) Int J Cancer, 82(4): 504-11 |
| Bc12 | Koty et al. (1999) Lung Cancer, 23(2): 115-127 |
| bcr-abl (b3a2) | Verfaillie et al. (\grave{ }996) Blood, 87(11): 4770-4779 |
| CA-125 | Bast et al. (\grave{ }998) Int J Biol Markers, 13(4): 179-187 |
| CASP-8/FLICE | Mandruzzato et al. (1997) J Exp Med., 186(5): 785-793. |
| Cathepsins | Thomssen et al. (1995) Clin Cancer Res., 1(7): 741-746 |
| CD19 | Scheuermann et al. (1995) Leuk Lymphoma, 18(5-6): 385-397 |
| CD20 | Knox et al. (1996) Clin Cancer Res., 2(3): 457-470 |
| CD21, CD23 | Shubinsky et al. (1997) Leuk Lymphoma, 25(5-6): 521-530 |
| CD22, CD38 | French et al. (1995) Br J Cancer, 71(5): 986-994 |
| CD33 | Nakase et al. (1996) Am J Clin Pathol., 105(6): 761-768 |
| CD35 | Yamakawa et al. Cancer, 73(11): 2808-2817 |
| CD44 | Naot et al. (1997) Adv Cancer Res., 71: 241-319 |
| CD45 | Buzzi et al. (1992) Cancer Res., 52(14): 4027-4035 |
| CD46 | Yamakawa et al. (1994) Cancer, 73(11): 2808-2817 |
| CD5 | Stein et al. (1991) Clin Exp Immunol., 85(3): 418-423 |
| CD52 | Ginaldi et al. (1998) Leuk Res., 22(2): 185-191 |
| CD55 | Spendlove et al. (1999) Cancer Res., 59: 2282-2286. |
| CD59 (791Tgp72) | Jarvis et al. (1997) Int J Cancer, 71(6): 1049-1055 |
| CDC27 | Wang et al. (1999) Science, 284(5418): 1351-1354 |
| CDK4 | Wölfel et al. (1995) Science, 269(5228): 1281-1284 |
| CEA | Kass et al. (1999) Cancer Res., 59(3): 676-683 |
| c-myc | Watson et al. (1991) Cancer Res., 51(15): 3996-4000 |
| Cox-2 | Tsujii et al. (1998) Cell, 93: 705-716 |
| DCC | Gotley et al. (1996) Oncogene, 13(4): 787-795 |
| DcR3 | Pitti et al. (1998) Nature, 396: 699-703 |
| E6/E7 | Steller et al. (1996) Cancer Res., 56(21): 5087-5091 |
| EGFR | Yang et al. (1999) Cancer Res., 59(6): 1236-1243. |
| EMBP | Shiina et al. (1996) Prostate, 29(3): 169-176. |
| Ena78 | Arenberg et al. (1998) J. Clin. Invest., 102: 465-472. |
| FGF8b and FGF8a | Dorkin et al. (1999) Oncogene, 18(17): 2755-2761 |
| FLK-1/KDR | Annie and Fong (1999) Cancer Res., 59: 99-106 |
| Folic Acid Receptor | Dixon et al. (1992) J Biol Chem., 267(33): 24140-72414 |
| G250 | Divgi et al. (1998) Clin Cancer Res., 4(11): 2729-2739 |
| GAGE-Family | De Backer et al. (1999) Cancer Res., 59(13): 3157-3165 |
| gastrin 17 | Watson et al. (1995) Int J Cancer, 61(2): 233-240 |
| Gastrin-releasing | Wang et al. (1996) Int J Cancer, 68(4): 528-534 |
| hormone (bombesin) | |
| GD2/GD3/GM2 | Wiesner and Sweeley (1995) Int J Cancer, 60(3): 294-299 |
| GnRH | Bahk et al. (1998) Urol Res., 26(4): 259-264 |
| GnTV | Hengstler et al. (1998) Recent Results Cancer Res., 154: 47-85 |
| gp100/Pmel17 | Wagner et al. (1997) Cancer Immunol Immunother., 44(4): 239- |
| | 247 |
| gp-100-in4 | Kirkin et al. (1998) APMIS, 106(7): 665-679 |
| gp15 | Maeurer et al. (1996) Melanoma Res., 6(1): 11-24 |
| gp75/TRP-1 | Lewis et al. (1995) Semin Cancer Biol., 6(6): 321-327 |
| hCG | Hoermann et al. (1992) Cancer Res., 52(6): 1520-1524 |
| Heparanase | Vlodavsky et al. (1999) Nat Med., 5(7): 793-802 |
| Her2/neu | Lewis et al. (1995) Semin Cancer Biol., 6(6): 321-327 |
| Her3 | |
| HMTV | Kahl et al. (1991) Br J Cancer, 63(4): 534-540 |
| Hsp70 | Jaattela et al. (1998) EMBO J., 17(21): 6124-6134 |
| hTERT | Vonderheide et al. (1999) Immunity, 10: 673-679. 1999. |
| (telomerase) | |
| IGFR1 | Ellis et al. (1998) Breast Cancer Res. Treat., 52: 175-184 |
| IL-13R | Murata et al. (1997) Biochem Biophys Res Commun., 238(1): 90-94 |
| iNOS | Klotz et al. (1998) Cancer, 82(10): 1897-1903 |
| Ki 67 | Gerdes et al. (1983) Int J Cancer, 31: 13-20 |
| KIAA0205 | Guéguen et al. (1998) J Immunol., 160(12): 6188-6194 |
| K-ras, H-ras, | Abrams et al. (1996) Semin Oncol., 23(1): 118-134 |
| N-ras | |
| KSA | Zhang et al. (1998) Clin Cancer Res., 4(2): 295-302 |
| (CO17-1A) | |
| LDLR-FUT | Caruso et al. (1998) Oncol Rep., 5(4): 927-930 |
| MAGE Family | Marchand et al. (1999) Int J Cancer, 80(2): 219-230 |
| (MAGE1, | |
| MAGE3, etc.) | |
| Mammaglobin | Watson et al. (1999) Cancer Res., 59: 13 3028-3031 |
| MAP17 | Kocher et al. (1996) Am J Pathol., 149(2): 493-500 |
| Melan-A/ | Lewis and Houghton (1995) Semin Cancer Biol., 6(6): 321-327 |
| MART-1 | |
| mesothelin | Chang et al. (1996) Proc. Natl. Acad. Sci., USA, 93(1): 136-140 |
| MIC A/B | Groh et al. (1998) Science, 279: 1737-1740 |
| MT-MMP's, such as | Sato and Seiki (1996) J Biochem (Tokyo), 119(2): 209-215 |
| MMP2, MMP3, | |
| MMP7, MMP9 | |
| Mox1 | Candia et al. (1992) Development, 116(4): 1123-1136 |
| Mucin, such as MUC- | Lewis and Houghton (1995) Semin Cancer Biol., 6(6): 321-327 |
| 1, MUC-2, MUC-3, | |
| and MUC-4 | |
| MUM-1 | Kirkin et al. (1998) APMIS, 106(7): 665-679 |
| NY-ESO-1 | Jager et al. (1998) J. Exp. Med., 187: 265-270 |
| Osteonectin | Graham et al. (1997) Eur J Cancer, 33(10): 1654-1660 |
| p15 | Yoshida et al. (1995) Cancer Res., 55(13): 2756-2760 |
| P170/MDR1 | Trock et al. (1997) J Natl Cancer Inst., 89(13): 917-931 |
| p53 | Roth et al. (1996) Proc. Natl. Acad. Sci., USA, 93(10): 4781-4786. |
| p97/melanotransferrin | Furukawa et al. (1989) J Exp Med., 169(2): 585-590 |
| PAI-1 | Grøndahl-Hansen et al. (1993) Cancer Res., 53(11): 2513-2521 |
| PDGF | Vassbotn et al. (1993) Mol Cell Biol., 13(7): 4066-4076 |
| Plasminogen (uPA) | Naitoh et al. (1995) Jpn J Cancer Res., 86(1): 48-56 |
| PRAME | Kirkin et al. (1998) APMIS, 106(7): 665-679 |
| Probasin | Matuo et al. (1985) Biochem Biophys Res Commun., 130(1): 293- |
| | 300 |
| Progenipoietin | — |
| PSA | Sanda et al. (1999) Urology, 53(2): 260-266. |
| PSM | Kawakami et al. (1997) Cancer Res., 57(12): 2321-2324 |
| RAGE-1 | Gaugler et al. (1996) Immunogenetics, 44(5): 323-330 |
| Rb | Dosaka-Akita et al. (1997) Cancer, 79(7): 1329-1337 |
| RCAS1 | Sonoda et al. (1996) Cancer, 77(8): 1501-1509. |
| SART-1 | Kikuchi et al. (1999(Int J Cancer, 81(3): 459-466 |
| SSX gene | Gure et al. (1997) Int J Cancer, 72(6): 965-971 |
| Family | |
| STAT3 | Bromberg et al. (1999) Cell, 98(3): 295-303 |
| STn | Sandmaier et al. (1999) J Immunother., 22(1): 54-66 |
| (mucin assoc.) | |
| TAG-72 | Kuroki et al. (1990)Cancer Res., 50(16): 4872-4879 |
| TGF-α | Imanishi et al. (1989) Br J Cancer, 59(5): 761-765 |
| TGF-β | Picon et al. (1998) Cancer Epidemiol Biomarkers Prey, 7(6): 497- |
| | 504 |
| Thymosin β 15 | Bao et al. (1996) Nature Medicine. 2(12), 1322-1328 |
| IFN-α | Moradi et al. (1993) Cancer, 72(8): 2433-2440 |
| TPA | Maulard et al. (1994) Cancer, 73(2): 394-398 |
| TPI | Nishida et al. (1984) Cancer Res 44(8): 3324-9 |
| TRP-2 | Parkhurst et al. (1998) Cancer Res., 58(21) 4895-4901 |
| Tyrosinase | Kirkin et al. (1998) APMIS, 106(7): 665-679 |
| VEGF | Hyodo et al. (1998) Eur J Cancer, 34(13): 2041-2045 |
| ZAG | Sanchez et al. (1999) Science, 283(5409): 1914-1919 |
| p16INK4 | Quelle et al. (1995) Oncogene Aug. 17, 1995; 11(4): 635-645 |
| Glutathione | Hengstler (1998) et al. Recent Results Cancer Res., 154: 47-85 |
| S-transferase | |

Any of the foregoing markers can be used as targets for the targeting moieties comprising delivery vehicles described herein. In certain embodiments the target markers include, but are not limited to members of the epidermal growth factor family (e.g., HER2, HER3, EGF, HER4), CD1, CD2, CD3, CD5, CD7, CD13, CD14, CD15, CD19, CD20, CD21, CD23, CD25, CD33, CD34, CD38, 5E10, CEA, HLA-DR, HM 1.24, HMB 45, 1a, Leu-M1, MUC1, PMSA, TAG-72, phosphatidyl serine antigen, and the like.

The foregoing markers are intended to be illustrative and not limiting. Other tumor associated antigens will be known to those of skill in the art.

Where the tumor marker is a cell surface receptor, a ligand to that receptor can function as targeting moieties. Similarly, mimetics of such ligands can also be used as targeting moieties. Thus, in certain embodiments peptide ligands, and other ligands, can be used in addition to or in place of various antibodies. An illustrative, but non-limiting list of suitable targeting ligands is shown in Table 3. In certain embodiments any one or more of these peptides can be conjugated to a drug delivery vehicle described herein.

TABLE 3
Illustrative, but non-limiting ligands that target membrane
receptors expressed or overexpressed by various cancer cells.

| Target Membrane | Targeting | SEQ ID |
|---|---|---|
| Receptor | ligand | NO |
| Integrin receptor Aᵥβ₃ | c(RGDfK) | 2 |
| | c(RGDfC) | 3 |
| | c(RGDyC) | 4 |
| | RGD | |
| GFR | Peptide GE11 | 5 |
| | (YHWYGYTPQNVI) | |
| GFR | GSG-KCCYSL | 6 |
| SSTR2 | Ostreotide | |
| GRP | QWAVGHML | 7 |
| CCK | DYMGWMDF | 8 |
| NT | RRPYIL | 9 |
| | RRPYILQLYENKPRRPYIL | 10 |
| LHRH | Gondaorelin | |
| GPRC family members | Antagonist G | |
| Tumor Cell | Targeting | |
| Receptor | Ligand | |
| TfR | Tf | |
| EGFR | EGF | |
| FAR (FR-α) | FA | |
| FR-α | Methotrexate | |
| Sigma receptor | Anisamide | |
| Importing α and β receptors | TAT peptides | |
| IL-13Rα2 | IL-13 peptide | |
| HER2 | Anti-herceptin | |
| HER2/neu | Anti-HER2/neu | |
| ErbB2 | Anti-ErbB2 | |
| Mesothelin | Anti-ME1 | |
| CD105/endoglin | Anti-TRC105 | |
| NET | MABG | |
| NRP-1 | RGD-type peptide (RDGRC) | 11 |
| SA | ConA | |
| CD44 | HA | |
| αᵥβ₃ integrins | c(RGDyK) | |
| αᵥβ₃ integrins | cRGD | |
| αᵥβ₃ integrins | K₇RGD | 12 |
| | c-RGDFK | 13 |
| αᵥβ₃ integrins | K₈(RGD)₂ | 14 |
| αᵥβ₃ integrins | N₃GPLGRGRGDK-Ad | 15 |
| αᵥβ₃ integrins | N₃RGDFFFFC | 16 |
| αᵥβ₃ integrins | Thiolated-RGD | |
| (VCAM-1)R | Anti-(VCAM-1) | |
| VEGFR | VEGF | |

Tf: Transferrin;
FA: Folic acid;
EGFR: Epidermal growth factor;
TAT: Transactivator of transcription;
IL-13: Interleukin-13;
MABG: metaaminobenzyl guanidine (meta-iodobenzylguanidine analogue);
ConA: concanavalin A;
c(RGD): Cyclic RGD (Arg-Gly-Asp);
c(RGDyK): Cyclo(Arg-Gly-Asp-D-Phe-Lys);
K₇RGD: linear RGD peptide sequence with 7 consecutive lysine residues;
K₈(RGD)₂ cationic peptide containing 2 RGD sequences;
VCAM-1: vascular cell adhesion molecule 1;
VEGFR: Vascular endothelial growth factor;
TfR: transferrin receptor;
EGFR: epidermal growth factor receptor;
FAR (FR-α): Folic acid receptor;
IL-13Rα2: interleukin-13 receptor subunit alpha-2;
HER2: epidermal growth factor receptor;
ErbB2: Receptor tyrosine-protein kinase 2;
NET: norepinephrine transporter;
NRP-1: neuropilin receptors;
SA: sialic acid;
(VCAM-1)R: vascular cell adhesion molecule 1 receptor;
VEGFR: Vascular endothelial growth factor receptor
c( ) indicates cyclopeptide.
Lower case indicates “D” amino acid.

In certain embodiments the nanoparticle drug delivery vehicles described herein can be conjugated to moieties that facilitate stability in circulation and/or that hide the drug delivery vehicle from the reticuloendothelial system (RES) and/or that facilitate transport across a barrier (e.g., a stromal barrier, the blood brain barrier, etc.), and/or into a tissue. In certain embodiments the drug delivery vehicle is conjugated to transferrin or ApoE to facilitate transport across the blood brain barrier. In certain embodiments the drug delivery vehicle is conjugated to folate.

Methods of coupling the nanoparticle drug delivery vehicle to targeting (or other) agents are well known to those of skill in the art. Examples include, but are not limited to the use of biotin and avidin or streptavidin (see, e.g., U.S. Pat. No. 4,885,172 A), by traditional chemical reactions using, for example, bifunctional coupling agents such as glutaraldehyde, diimide esters, aromatic and aliphatic diisocyanates, bis-p-nitrophenyl esters of dicarboxylic acids, aromatic disulfonyl chlorides and bifunctional arylhalides such as 1,5-difluoro-2,4-dinitrobenzene; p,p′-difluoro m,m′-dinitrodiphenyl sulfone, sulfhydryl-reactive maleimides, and the like. Appropriate reactions which may be applied to such couplings are described in Williams et al. Methods in Immunology and Immunochemistry Vol. 1, Academic Press, New York 1967.

In one illustrative but non-limiting approach a peptide (e.g., iRGD) is coupled to the nanoparticle drug delivery vehicle by a lipid coupled to a linker (e.g., DSPE-PEG₂₀₀₀-maleimide), allowing thiol-maleimide coupling to the cysteine-modified peptide. It will also be recognized that in certain embodiments the targeting (and other) moieties can be conjugated to other moieties comprising the lipid bilayer. In certain embodiments possible to improve tumor delivery of the Pt-based drug loaded nanoparticle through co-administration (not conjugated) of the iRGD peptide to enhance particle transcytosis.

The former conjugates and coupling methods are illustrative and non-limiting. Using the teachings provided herein, numerous other moieties can be conjugated to the nanoparticle drug delivery vehicles described herein by any of a variety of methods.

### Pharmaceutical Formulations, Administration and Therapy

In some embodiments, the nanoparticle drug delivery vehicles described herein are administered alone or in a mixture with a physiologically-acceptable carrier (such as physiological saline or phosphate buffer) selected in accordance with the route of administration and standard pharmaceutical practice. For example, when used as an injectable, the nanoparticle drug delivery vehicles can be formulated as a sterile suspension, dispersion, or emulsion with a pharmaceutically acceptable carrier. In certain embodiments normal saline can be employed as the pharmaceutically acceptable carrier. Other suitable carriers include, e.g., water, buffered water, 0.4% saline, 0.3% glycine, 5% glucose and the like, including glycoproteins for enhanced stability, such as albumin, lipoprotein, globulin, etc. In compositions comprising saline or other salt-containing carriers, the carrier is preferably added following nanoparticle drug delivery vehicle formation. Thus, after the nanoparticle drug delivery vehicle is formed and loaded with suitable drug(s), the vehicles can be diluted into pharmaceutically acceptable carriers such as normal saline.

The pharmaceutical compositions may be sterilized by conventional, well-known sterilization techniques. The resulting aqueous solutions, suspensions, dispersions, emulsions, etc., may be packaged for use or filtered under aseptic conditions. In certain embodiments the nanoparticle drug delivery vehicles described herein are lyophilized, the lyophilized preparation being combined with a sterile aqueous solution prior to administration. The compositions may also contain pharmaceutically acceptable auxiliary substances as required to approximate physiological conditions, such as pH-adjusting and buffering agents, tonicity adjusting agents and the like, for example, sodium acetate, sodium lactate, sodium chloride, potassium chloride, calcium chloride, etc.

Additionally, in certain embodiments, the pharmaceutical formulation may include lipid-protective agents that protect lipids against free-radical and lipid-peroxidative damage on storage. Lipophilic free-radical quenchers, such as alpha-tocopherol and water-soluble iron-specific chelators, such as ferrioxamine, are suitable.

The concentration of the nanoparticle drug delivery vehicles in the pharmaceutical formulations can vary widely, e.g., from less than approximately 0.05%, usually at least approximately 2 to 5% to as much as 10 to 50%, or to 40%, or to 30% by weight and are selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, nanoparticle drug delivery vehicles composed of irritating lipids may be diluted to low concentrations to lessen inflammation at the site of administration. The amount of nanoparticle drug delivery vehicles administered will depend upon the particular drug used, the disease state being treated and the judgment of the clinician but will generally be between approximately 0.01 and approximately 50 mg per kilogram of body weight, preferably between approximately 0.1 and approximately 5 mg per kg of body weight.

In some embodiments, it is desirable to include polyethylene glycol (PEG)-modified phospholipids in the LB-coated nanoparticles or vesicles. Alternatively, or additionally, in certain embodiments, PEG-ceramide, or ganglioside G_{MI}-modified lipids can be incorporated in the nanoparticle drug delivery vehicles described herein. Addition of such components helps prevent delivery vehicle aggregation and provides for increasing circulation lifetime and increasing the delivery of the loaded delivery vehicles to the target tissues.

In some embodiments, overall nanoparticle drug delivery vehicle charge is an important determinant in clearance of the vehicle from the blood. It is believed that highly charged delivery vehicles (e.g., zeta potential >+35 mV) will be typically taken up more rapidly by the reticuloendothelial system (see, e.g., Juliano (1975), Biochem. Biophys. Res. Commun. 63: 651-658 discussing liposome clearance by the RES). Drug delivery vehicles with prolonged circulation half-lives are typically desirable for therapeutic uses. For instance, in certain embodiments, drug delivery nanoparticle drug delivery vehicles that are maintained from 8 hrs, or 12 hrs, or 24 hrs, or greater are desirable.

In another example of their use, the nanoparticle drug delivery vehicles can be incorporated into a broad range of topical dosage forms including but not limited to gels, oils, emulsions, and the like, e.g., for the treatment of a topical cancer. For instance, in some embodiments the suspension containing the drug delivery vehicles is formulated and administered as a topical cream, paste, ointment, gel, lotion, and the like.

In some embodiments, pharmaceutical formulations comprising the nanoparticle drug delivery vehicles described herein additionally incorporate a buffering agent. The buffering agent may be any pharmaceutically acceptable buffering agent. Buffer systems include, but are not limited to citrate buffers, acetate buffers, borate buffers, and phosphate buffers. Examples of buffers include, but are not limited to citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate, benzoic acid, and the like.

In some embodiments, pharmaceutical formulations comprising the nanoparticle drug delivery vehicles described herein additionally incorporate a chelating agent. The chelating agent may be any pharmaceutically acceptable chelating agent. Chelating agents include, but are not limited to ethylene diaminetetraacetic acid (also synonymous with EDTA, edetic acid, versene acid, and sequestrene), and EDTA derivatives, such as dipotassium edetate, disodium edetate, edetate calcium disodium, sodium edetate, trisodium edetate, and potassium edetate. Other chelating agents include citric acid (e.g., citric acid monohydrate) and derivatives thereof. Derivatives of citric acid include anhydrous citric acid, trisodiumcitrate-dihydrate, and the like. Still other chelating agents include, but are not limited to, niacinamide and derivatives thereof and sodium deoxycholate and derivatives thereof.

In some embodiments, pharmaceutical formulations comprising the nanoparticle drug delivery vehicles described herein additionally incorporate an antioxidant. The antioxidant may be any pharmaceutically acceptable antioxidant. Antioxidants are well known to those of ordinary skill in the art and include, but are not limited to, materials such as ascorbic acid, ascorbic acid derivatives (e.g., ascorbylpalmitate, ascorbylstearate, sodium ascorbate, calcium ascorbate, etc.), butylated hydroxy anisole, buylated hydroxy toluene, alkylgallate, sodium meta-bisulfate, sodium bisulfate, sodium dithionite, sodium thioglycollic acid, sodium formaldehyde sulfoxylate, tocopherol and derivatives thereof, (d-alpha tocopherol, d-alpha tocopherol acetate, dl-alpha tocopherol acetate, d-alpha tocopherol succinate, beta tocopherol, delta tocopherol, gamma tocopherol, and d-alpha tocopherol polyoxyethylene glycol 1000 succinate) monothioglycerol, sodium sulfite and N-acetyl cysteine. In certain embodiments such materials, when present, are typically added in ranges from 0.01 to 2.0%.

In some embodiments, pharmaceutical formulations comprising the nanoparticle drug delivery vehicles described herein are formulated with a cryoprotectant. The cryoprotecting agent may be any pharmaceutically acceptable cryoprotecting agent. Common cryoprotecting agents include, but are not limited to, histidine, polyethylene glycol, polyvinyl pyrrolidine, lactose, sucrose, mannitol, polyols, and the like.

In some embodiments, pharmaceutical formulations comprising the nanoparticle drug delivery vehicles described herein are formulated with an isotonic agent. The isotonic agent can be any pharmaceutically acceptable isotonic agent. This term is used in the art interchangeably with iso-osmotic agent, and is known as a compound that is added to the pharmaceutical preparation to increase the osmotic pressure, e.g., in some embodiments to that of 0.9% sodium chloride solution, which is iso-osmotic with human extracellular fluids, such as plasma. Illustrative isotonicity agents include, but are not limited to, sodium chloride, mannitol, sorbitol, lactose, dextrose and glycerol.

In certain embodiments pharmaceutical formulations of the the nanoparticle drug delivery vehicles described herein may optionally comprise a preservative. Common preservatives include, but are not limited to, those selected from the group consisting of chlorobutanol, parabens, thimerosol, benzyl alcohol, and phenol. Suitable preservatives include but are not limited to: chlorobutanol (e.g., 0.3-0.9% w/v), parabens (e.g., 0.01-5.0%), thimerosal (e.g., 0.004-0.2%), benzyl alcohol (e.g., 0.5-5%), phenol (e.g., 0.1-1.0%), and the like.

In some embodiments, pharmaceutical formulations comprising the nanoparticle drug delivery vehicles described herein are formulated with a humectant, e.g., to provide a pleasant mouth-feel in oral applications. Humectants known in the art include, but are not limited to, cholesterol, fatty acids, glycerin, lauric acid, magnesium stearate, pentaerythritol, and propylene glycol.

In some embodiments, an emulsifying agent is included in the formulations, for example, to ensure complete dissolution of all excipients, especially hydrophobic components such as benzyl alcohol. Many emulsifiers are known in the art, e.g., polysorbate 60.

For some embodiments related to oral administration, it may be desirable to add a pharmaceutically acceptable flavoring agent and/or sweetener. Compounds such as saccharin, glycerin, simple syrup, and sorbitol are useful as sweeteners.

Administration

The nanoparticle drug delivery vehicles described herein can be administered to a subject (e.g., patient) by any of a variety of techniques.

In certain embodiments the nanoparticle drug delivery vehicles and/or pharmaceutical formulations thereof are administered parenterally, e.g., intraarticularly, intravenously, intraperitoneally, subcutaneously, or intramuscularly. In some embodiments, the pharmaceutical compositions are administered intravenously, intraarterially, or intraperitoneally by a bolus injection (see, e.g., U.S. Pat. Nos. 3,993,754; 4,145,410; 4,235,871; 4,224,179; 4,522,803; and 4,588,578 describing administration of liposomes). Particular pharmaceutical formulations suitable for this administration are found in Remington's Pharmaceutical Sciences, Mack Publishing Company, Philadelphia, Pa., 17th ed. (1985). Typically, the formulations comprise a solution of the nanoparticle drug delivery vehicles suspended in an acceptable carrier, preferably an aqueous carrier. As noted above, suitable aqueous solutions include, but are not limited to physiologically compatible buffers such as Hanks solution, Ringer's solution, or physiological (e.g., 0.9% isotonic) saline buffer and/or in certain emulsion formulations. The solution(s) can contain formulatory agents such as suspending, stabilizing and/or dispersing agents. In certain embodiments the active agent(s) can be provided in powder form for constitution with a suitable vehicle, e.g., sterile pyrogen-free water, before use. For transmucosal administration, and/or for blood/brain barrier passage, penetrants appropriate to the barrier to be permeated can be used in the formulation. These compositions may be sterilized by conventional, well-known sterilization techniques, or may be sterile filtered. The resulting aqueous solutions may be packaged for use as is, or lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc., e.g., as described above.

In other methods, the pharmaceutical formulations containing the nanoparticle drug delivery vehicles described herein may be contacted with the target tissue by direct application of the preparation to the tissue. The application may be made by topical, “open” or “closed” procedures. By “topical” it is meant the direct application of the pharmaceutical preparation to a tissue exposed to the environment, such as the skin, oropharynx, external auditory canal, and the like. Open procedures are those procedures that include incising the skin of a patient and directly visualizing the underlying tissue to which the pharmaceutical formulations are applied. This is generally accomplished by a surgical procedure, such as a thoracotomy to access the lungs, abdominal laparotomy to access abdominal viscera, or other direct surgical approaches to the target tissue. Closed procedures are invasive procedures in which the internal target tissues are not directly visualized, but accessed via inserting instruments through small wounds in the skin. For example, the preparations may be administered to the peritoneum by needle lavage. Likewise, the pharmaceutical preparations may be administered to the meninges or spinal cord by infusion during a lumbar puncture followed by appropriate positioning of the patient as commonly practiced for spinal anesthesia or metrizamide imaging of the spinal cord. Alternatively, the preparations may be administered through endoscopic devices. In certain embodiments the pharmaceutical formulations are introduced via a cannula.

In certain embodiments the pharmaceutical formulations comprising the nanoparticle drug delivery vehicles described herein are administered via inhalation (e.g., as an aerosol). Inhalation can be a particularly effective delivery route for administration to the lungs and/or to the brain. For administration by inhalation, the nanoparticle drug delivery vehicles are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator may be formulated containing a powder mix of the compound and a suitable powder base such as lactose or starch.

In certain embodiments, the nanoparticle drug delivery vehicles described herein are formulated for oral administration. For oral administration, suitable formulations can be readily formulated by combining the drug delivery vehicles with pharmaceutically acceptable carriers suitable for oral delivery well known in the art. Such carriers enable the active agent(s) described herein to be formulated as tablets, pills, dragees, caplets, lozenges, gelcaps, capsules, liquids, gels, syrups, slurries, suspensions and the like, for oral ingestion by a patient to be treated. For oral solid formulations such as, for example, powders, capsules and tablets, suitable excipients can include fillers such as sugars (e.g., lactose, sucrose, mannitol and sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose), synthetic polymers (e.g., polyvinylpyrrolidone (PVP)), granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques. The preparation of enteric-coated particles is disclosed for example in U.S. Pat. Nos. 4,786,505 and 4,853,230.

In various embodiments the nanoparticle drug delivery vehicles described herein can be formulated in rectal or vaginal compositions such as suppositories or retention enemas, e.g., containing conventional suppository bases such as cocoa butter or other glycerides. Methods of formulating active agents for rectal or vaginal delivery are well known to those of skill in the art (see, e.g., Allen (2007) Suppositories, Pharmaceutical Press) and typically involve combining the active agents with a suitable base (e.g., hydrophilic (PEG), lipophilic materials such as cocoa butter or Witepsol W45), amphiphilic materials such as Suppocire AP and polyglycolized glyceride, and the like). The base is selected and compounded for a desired melting/delivery profile.

The route of delivery of the nanoparticle drug delivery vehicles described herein can also affect their distribution in the body. Passive delivery of the drug delivery vehicles involves the use of various routes of administration e.g., parenterally, although other effective administration forms, such as intraarticular injection, inhalant mists, orally active formulations, transdermal iontophoresis, or suppositories are also envisioned. Each route produces differences in localization of the drug delivery vehicle.

Because dosage regimens for pharmaceutical agents are well known to medical practitioners, the amount of the liposomal pharmaceutical agent formulations that is effective or therapeutic for the treatment of a disease or condition in mammals and particularly in humans will be apparent to those skilled in the art. The optimal quantity and spacing of individual dosages of the formulations herein will be determined by the nature and extent of the condition being treated, the form, route and site of administration, and the particular patient being treated, and such optima can be determined by conventional techniques. It will also be appreciated by one of skill in the art that the optimal course of treatment, e.g., the number of doses given per day for a defined number of days, can be ascertained by those skilled in the art using conventional course of treatment determination tests.

Typically, the nanoparticle drug delivery vehicles described herein and/or pharmaceutical formations thereof described herein are used therapeutically in animals (including man) in the treatment of various cancers. In certain embodiments the drug delivery vehicles and/or pharmaceutical formations thereof described herein are particularly well suited in conditions that require: (1) repeated administrations; and/or (2) the sustained delivery of the drug in its bioactive form; and/or (3) the decreased toxicity with suitable efficacy compared with the free drug(s) in question. In various embodiments the nanoparticle drug delivery vehicles and/or pharmaceutical formations thereof are administered in a therapeutically effective dose. The term “therapeutically effective” as it pertains to the nanoparticle drug delivery vehicles described herein and formulations thereof means that the metal-based drug(s) (e.g., platinum-based chemotherapeutic agents) inhibitor contained therein, alone or in combination with other drugs, produces a desirable effect on the cancer. Such desirable effects include, but are not limited to slowing and/or stopping tumor growth and/or proliferation and/or slowing and/or stopping proliferation of metastatic cells, reduction in size and/or number of tumors, and/or elimination of tumor cells and/or metastatic cells, and/or prevention of recurrence of the cancer following remission.

Exact dosages will vary depending upon such factors as the particular metal-based drug and the desirable medical effect, as well as patient factors such as age, sex, general condition, and the like. Those of skill in the art can readily take these factors into account and use them to establish effective therapeutic concentrations without resort to undue experimentation.

For administration to humans (or to non-human mammals) in the curative, remissive, retardive, or prophylactic treatment of diseases the prescribing physician will ultimately determine the appropriate dosage of the drug for a given human (or non-human) subject, and this can be expected to vary according to the age, weight, and response of the individual as well as the nature and severity of the patient's disease. In certain embodiments the dosage of the drug provided by the nanoparticle drug delivery vehicles can be approximately equal to that employed for the free drug. However as noted above, the nanoparticle drug delivery vehicles described herein can significantly reduce the toxicity of the drug(s) administered thereby and significantly increase a therapeutic window. Accordingly, in some cases dosages in excess of those prescribed for the free drug(s) will be utilized.

In certain embodiments, the dose of each of the drug(s) (e.g., PT-based drugs)) administered at a particular time point will be in the range from about 1 to about 1,000 mg/m²/day, or to about 800 mg/m²/day, or to about 600 mg/m²/day, or to about 400 mg/m²/day. For example, in certain embodiments a dosage (dosage regiment) is utilized that provides a range from about 1 to about 350 mg/m²/day, 1 to about 300 mg/m²/day, 1 to about 250 mg/m²/day, 1 to about 200 mg/m²/day, 1 to about 150 mg/m²/day, 1 to about 100 mg/m²/day, from about 5 to about 80 mg/m²/day, from about 5 to about 70 mg/m²/day, from about 5 to about 60 mg/m²/day, from about 5 to about 50 mg/m²/day, from about 5 to about 40 mg/m²/day, from about 5 to about 20 mg/m²/day, from about 10 to about 80 mg/m²/day, from about 10 to about 70 mg/m²/day, from about 10 to about 60 mg/m²/day, from about 10 to about 50 mg/m²/day, from about 10 to about 40 mg/m²/day, from about 10 to about 20 mg/m²/day, from about 20 to about 40 mg/m²/day, from about 20 to about 50 mg/m²/day, from about 20 to about 90 mg/m²/day, from about 30 to about 80 mg/m²/day, from about 40 to about 90 mg/m²/day, from about 40 to about 100 mg/m²/day, from about 80 to about 150 mg/m²/day, from about 80 to about 140 mg/m²/day, from about 80 to about 135 mg/m²/day, from about 80 to about 130 mg/m²/day, from about 80 to about 120 mg/m²/day, from about 85 to about 140 mg/m²/day, from about 85 to about 135 mg/m²/day, from about 85 to about 135 mg/m²/day, from about 85 to about 130 mg/m²/day, or from about 85 to about 120 mg/m²/day. In certain embodiments the does administered at a particular time point may also be about 130 mg/m²/day, about 120 mg/m²/day, about 100 mg/m²/day, about 90 mg/m²/day, about 85 mg/m²/day, about 80 mg/m²/day, about 70 mg/m²/day, about 60 mg/m²/day, about 50 mg/m²/day, about 40 mg/m²/day, about 30 mg/m²/day, about 20 mg/m²/day, about 15 mg/m²/day, or about 10 mg/m²/day.

In certain embodiments, the dose administered may be higher or lower than the dose ranges described herein, depending upon, among other factors, the bioavailability of the composition, the tolerance of the individual to adverse side effects, the mode of administration and various factors discussed above. Dosage amount and interval may be adjusted individually to provide plasma levels of the composition that are sufficient to maintain therapeutic effect, according to the judgment of the prescribing physician. Skilled artisans will be able to optimize effective local dosages without undue experimentation in view of the teaching provided herein.

Multiple doses (e.g., continuous or bolus) of the compositions as described herein may also be administered to individuals in need thereof of the course of hours, days, weeks, or months. For example, but not limited to, 1, 2, 3, 4, 5, or 6 times daily, every other day, every 10 days, weekly, monthly, twice weekly, three times a week, twice monthly, three times a month, four times a month, five times a month, every other month, every third month, every fourth month, etc.

### Methods of Treatment.

In various embodiments methods of treatment using the PT-drug loaded nanoparticle drug delivery vehicles described herein and/or pharmaceutical formulation(s) comprising the nanoparticle drug delivery vehicles described herein are provided. In certain embodiments the method(s) comprise a method of treating a cancer. In certain embodiments the method can comprise administering to a subject in need thereof an effective amount of a nanoparticle drug delivery vehicle described herein, and/or a pharmaceutical formulation comprising the nanoparticle drug delivery vehicles.

In certain embodiments the nanoparticle drug delivery vehicles described herein (containing one or more platinum-based drug(s)) and/or pharmaceutical formulation is a primary therapy in a chemotherapeutic regimen. In certain embodiments the nanoparticle drug delivery vehicle and/or pharmaceutical formulation is a component in an adjunct therapy in addition to chemotherapy using one or more other chemotherapeutic agents, and/or surgical resection of a tumor mass, and/or radiotherapy.

In certain embodiments the nanoparticle drug delivery vehicles and/or pharmaceutical formulation thereof is a component in a multi-drug chemotherapeutic regimen. In certain embodiments the multi-drug chemotherapeutic regimen comprises at least two drugs selected from the group consisting of irinotecan (IRIN), oxaliplatin (OX), 5-fluorouracil (5-FU), and leucovorin (LV). In certain embodiments the multi-drug chemotherapeutic regimen comprises at least three drugs selected from the group consisting of irinotecan (IRIN), oxaliplatin (OX), 5-fluorouracil (5-FU), and leucovorin (LV). In certain embodiments the multi-drug chemotherapeutic regimen comprises at least irinotecan (IRIN), oxaliplatin (OX), 5-fluorouracil (5-FU), and leucovorin (LV).

In various embodiments the nanoparticle drug delivery vehicles and/or pharmaceutical formulation(s) thereof described herein are effective for treating any of a variety of cancers. In certain embodiments the cancer is pancreatic ductal adenocarcinoma (PDAC). In certain embodiments the cancer is a cancer selected from the group consisting of acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, glioblastoma, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, chronic myeloid leukemia (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

In various embodiments, the Pt-drug loaded drug delivery vehicles (silicasomes) described herein are effective to treat a cancer that is routinely treated using a platinum-based therapeutic. Illustrative cancers include, but are not limited to pancreatic cancer, colorectal cancer, and cervical cancer.

In certain embodiments the nanoparticle drug delivery vehicles described herein are not conjugated to an iRGD peptide and the drug delivery vehicles are administered in conjunction with an iRGD peptide (e.g., the drug delivery vehicle and the iRGD peptide are co-administered as separate formulations).

In certain embodiments, the drug delivery vehicles described herein are administered as a component FOLFIRINOX protocol that additionally includes folinic acid, 5-fluorouracil, and irinotecan.

In certain embodiments, the drug delivery vehicles described herein are administered in conjunction with a checkpoint inhibitor (e.g., a PD-L1 inhibitor, a PD-1 inhibitor, a CTLA-4 inhibitor, etc.). In certain embodiments, the checkpoint inhibitor comprises one or more PD-L1 inhibitors. In certain embodiments, the checkpoint inhibitor comprises an anti-PD-L1 antibody. In certain embodiments, the checkpoint inhibitor comprises an anti-PD-L1 antibody selected from the group consisting of Atezolizumab, Avelumab, Durvalumab, BMS-936559, RG-7446. MPDL3280A, MEDI-4736, and MSB0010718C. In certain embodiments, the checkpoint inhibitor comprises a peptidic PD-L1 inhibitor. In certain embodiments, the PD-L1 inhibitor comprise a moiety selected from the group consisting of AUNP12, CA-170, and BMS-986189.

In certain embodiments, the checkpoint inhibitor comprises a PD1 inhibitor. In certain embodiments, the checkpoint inhibitor comprises an anti-PD1 antibody. In certain embodiments, the checkpoint inhibitor comprises an anti-PD1 antibody selected from the group consisting of Nivolumab, Pembrolizumab, Cemiplimab, avelumab, durvalumab, and atezolizumab.

In certain embodiments, the checkpoint inhibitor comprises an fc fusion with PD-L2. In certain embodiments, the checkpoint inhibitor comprises AMP224.

In certain embodiments, the checkpoint inhibitor comprises CTLA-4 inhibitor. In certain embodiments, the CTLA-4 inhibitor comprises Ipilimumab.

In certain embodiments, the checkpoint inhibitor comprises a bispecific antibody that binds to two checkpoint inhibitors, or an antibody that binds to a checkpoint inhibitor attached to a cytokine. In certain embodiments, the checkpoint inhibitor comprises a bispecific antibody that binds to two checkpoint inhibitors. In certain embodiments, the bispecific antibody comprises an antibody that binds to PD-1 attached to an antibody that binds to PD-L1, or an antibody that binds to PD-1 attached to an antibody that binds to CTLA4, or an antibody that binds to PD-L1 attached to an antibody that binds to CTLA4. In certain embodiments, the bispecific antibody comprises an antibody that binds to PD-1 attached to an antibody that binds to CTLA4. In certain embodiments, the checkpoint inhibitor comprises a cytokine attached to an antibody that binds to a checkpoint inhibitor. In certain embodiments, the checkpoint inhibitor comprises a cytokine attached to an antibody selected from the group consisting of anti-PD-1, anti-PD-L1, and CTLA4. In certain embodiments, the checkpoint inhibitor comprises cytokine attached to an anti-PD-1 antibody. In certain embodiments, the checkpoint inhibitor comprises an IL-7 attached to an anti-PD-1 antibody.

In various embodiments of these treatment methods, the Pt-based drug loaded nanoparticle drug delivery vehicles described herein and/or pharmaceutical formulations are administered via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition. In certain embodiments the drug delivery vehicles and/or pharmaceutical formulations thereof are administered as an injection, from an IV drip bag, or via a drug-delivery cannula. In various embodiments the subject is a human and in other embodiments the subject is a non-human mammal.

While the drug delivery vehicles described herein are often used in the treatment of cancer, depending on the metal-based drug(s) loaded into the vehicle the drug delivery vehicles find utility in a number of other indications such as autoimmune disease (e.g., rheumatoid arthritis), systemic bacterial, fungal, or viral infection, as imaging reagents, and the like.

### Kits.

In certain embodiments, kits are provided containing reagents for the practice of any of the methods described herein. In certain embodiments the kit comprises a container containing a drug delivery vehicle described herein.

Additionally, in certain embodiments, the kits can include instructional materials disclosing the means of the use of the nanoparticle drug delivery vehicles described herein as a cancer therapeutic.

In addition, the kits optionally include labeling and/or instructional materials providing directions (e.g., protocols) for the use of the materials described herein, e.g., alone or in combination for the treatment of various cancers. Instructional materials can also include recommended dosages, description(s) of counter indications, and the like.

While the instructional materials in the various kits typically comprise written or printed materials they are not limited to such. Any medium capable of storing such instructions and communicating them to an end user is contemplated by this invention. Such media include but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Development of Facile And Versatile Platinum Drug Delivering Silicasome Nanocarriers for Efficient Pancreatic Cancer Chemo-Immunotherapy

In this example we describe the development of a mesoporous silica nanoparticle (MSNP) based platform for high-dose loading of a range of activated platinum (Pt) chemo agents that could be attached to the porous interior through the use of electrostatic and coordination chemistry under weak-basic pH conditions. In addition to the design feature for improving drug delivery, the MSNP could also be encapsulated in a coated lipid bilayer (silicasome), to improve the colloidal stability after intravenous (IV) injection. We demonstrate improved pharmacokinetics and intratumor delivery of encapsulated oxaliplatin (DACHPt) over free drug in an orthotopic Kras-derived pancreatic cancer (PDAC) model. Not only did IV injection of the DACHPt silicasome provide more efficacious cytotoxic tumor cell killing, but could also demonstrate that chemotherapy-induced cell death is accompanied by the features of immunogenic cell death (ICD) as well as a dramatic reduction in bone marrow toxicity. The added features of an immunogenic response were reflected by calreticulin and HMGB1 expression, along with increased CD8⁺/FoxP3⁺ T-cell ratios and evidence of perforin and granzyme B release at the tumor site. Subsequent performance of a survival experiment demonstrated that the DACHPt silicasome generate a significant improved survival outcome, which could be extended by co-administration of an anti-PD-1 antibody.

### Results and Discussion

Three common Pt drug payloads were used in this study to develop our drug loading strategy, namely oxaliplatin, cisplatin and dichloro (ethylenediamine) platinum (Pt(en)Cl₂) (FIG. 1, panel A, structures 2-4)^{[7b]}. It is generally agreed upon that Pt drugs exist as an equilibrium of “neutral” or “cationic” species in an aqueous solution (FIG. 1, panel B)^{[16]}. The binding equilibrium is dependent on the Cl⁻ ion concentration (C_{Cl}_{^{−}}) as well as pH^{[16]}. While the neutral drug version is dominant in the blood circulation due to a high C_{Cl}_{^{−}} concentration (˜150 mM), the formation of an intracellular cationic version is facilitated due to a lower C_{Cl}_{^{−}} concentration (˜30 mM)^{[17]}. Moreover, the cationic formulation is regarded as pharmaceutically active due to the high reactivity of coordinated crosslinking to DNA, which stops cancer growth^{[16]}. These findings prompted us to consider loading cationic, activated Pt drugs into MSNP rather than working with pristine drugs. This requires the use of “neutral” Pt drugs where the X₂ligand is represented by Cl-ions. This required the purchasing of commercially available dichloro(1,2-diaminocyclohexane) platinum(II) (structure 5 in FIG. 1, panel C) as the neutral version of oxaliplatin, in addition to the use of cisplatin and Pt(en)Cl₂, which are already in the neutral form. This allowed us to proceed with the synthesis of 3 activated Pt drugs through the introduction of silver nitrate at 70° C., resulting in the formation of DACHPt (structure 6 in FIG. 1, panel C), DAPt (structure 7 in FIG. 1, panel C) and EDAPt (structure 8 in FIG. 1, panel C). These represent the activated versions of oxaliplatin, cisplatin and (Pt(en)Cl₂), respectively, and could be derived from high reaction yields (>95%). The reaction also generated silver chloride precipitates, which can be easily removed through centrifugation and filtration.

The synthesis procedure for deriving the Pt-silicasomes nanocarriers is schematically outlined in FIG. 2, panel A, which illustrates the following steps: (1) synthesis of bare MSNP, (2) soak-in of the activated Pt drugs under controlled pH conditions, (3) lipid coating, and (4) purification and sterilization. In order to successfully implement the design process shown in FIG. 2, panel A, it was necessary to develop a multi-parameter design process that takes into consideration pH, energy input, feed ratio and lipid coating (FIG. 2, panel A, inserted table). Sixty-five nm bare MSNP was synthesized using a sol-gel reaction, as previously reported^{[4b]}. MSNP is an amorphous silica nanoparticle in which the silica surface display of silanol groups^{[18]}. While our previous studies have delineated the possible types of silanol groups on MSNP surface^{[19]}, it is known that these silanol groups can be ionized at high pH condition, i.e. ≡Si—OH (low pH)⇔≡Si—O⁻ (high pH) (FIG. 2, panel B (B1)). Use of the ionized silanols, which exert most of the negative charge, are advantageous to accomplish attachment of the cationic Pt drugs via a combination of coordination and electrostatic interactions (FIG. 2, panel B (B2)). Thus, we asked whether simple pH fine-tuning could increase the abundance and efficiency of the soaked-in drug in MSNP. This demonstrated that incremental increase of the negative charge by pH adjustment from 5.5 to 8.5 was able to show increased drug binding efficiency on MSNPs (FIG. 2, panel C). During incubation of DACHPt with the MSNP to achieve a w/w coupling ratio of 1:5 for Pt:MSNP, we observed a significant improvement in encapsulation efficiency (EE %), to achieve ˜75% at pH 8.5. This was considerably higher than the EE at a weak acidic (pH 5.5) or neutral pH (7.4), which could only accomplish values of 25% and 60%, respectively (FIG. 2, panel C). In addition to pH, we also assessed the effect of stirring vs sonication, the soak-in time, and Pt:MSNP feed ratios to identify the optimal design parameters, as outlined in FIG. 8.

Following the synthesis of the DACHPt soaked-in bare particles, the MSNPs were subsequently coated by a lipid bilayer (LB), which were accomplished by an ethanol exchange method that results in the formation of a bilayer with a molar ratio of 3:2:0.15 for DSPC:cholesterol:PE-PEG2K, respectively^{[4b]}. The LB provided rapid and uniform pore sealing, capable of entrapping drug payloads of ˜70% into the porous interior (FIG. 9). Following experimentation with multi-parameter adjustment, it was possible through the use of ICP-MS Pt elemental analysis, to accomplish an EE % of ˜53% and a LC % of ˜21 wt % (e.q. oxaliplatin) for our optimized DACHPt silicasome formulation. We also prepared a control silicasome carrier that encapsulated oxaliplatin by using a passive loading approach. This demonstrated a ˜5× higher LC % and EE % values using the coordination approach, versus passive loading (FIG. 2, panel D).

In order to confirm the LC % analysis, we conducted elemental mapping at nanostructural level through performance of energy-dispersive X-ray spectroscopy (EDS) in combination with scanning transmission electron microscope (STEM) (FIG. 2, panel E). While STEM allowed visualization of the silicasome morphology, complementary EDS spectrum imaging of the same regions of interest (ROI) allowed high resolution visualization of the compositional variations. EDS, revealed a strong signature for Pt in the DACHPt silicasome compared to a weaker signal for the same element in the oxaliplatin silicasome (FIG. 2, panel E). This is in agreement with the EDS spectrum that showed a Pt/Si element ratio (w/w) of 19.4% for the DACHPt silicasome compared to a ratio of 3.3% for the in oxaliplatin silicasome. We also observed a phosphorus (P) signal in the EDS, which confirmed particle coating by a phospholipid containing bilayer.

In order to show the broader application of our loading approach, we also carried out drug loading studies for DAPt (activated cisplatin) and EDAPt (activated Pt(en)Cl₂) at pH 8.5, followed by lipid coating. ICP-MS analysis confirmed that the LC % and EE % were improved 5.6 to 8.5-fold, respectively, compared to passive drug loading (FIG. 2, panel F, upper panel). Improved drug loading was also confirmed by in situ STEM-EDS visualization (FIG. 2, panel F, bottom panel).

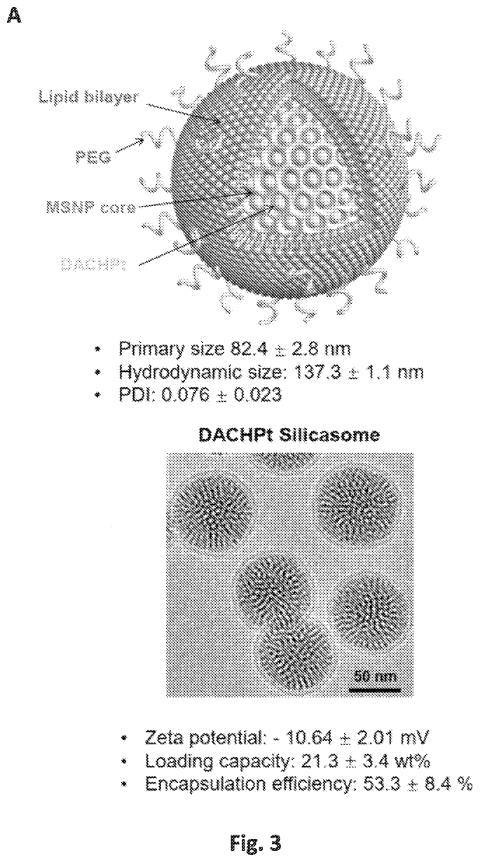

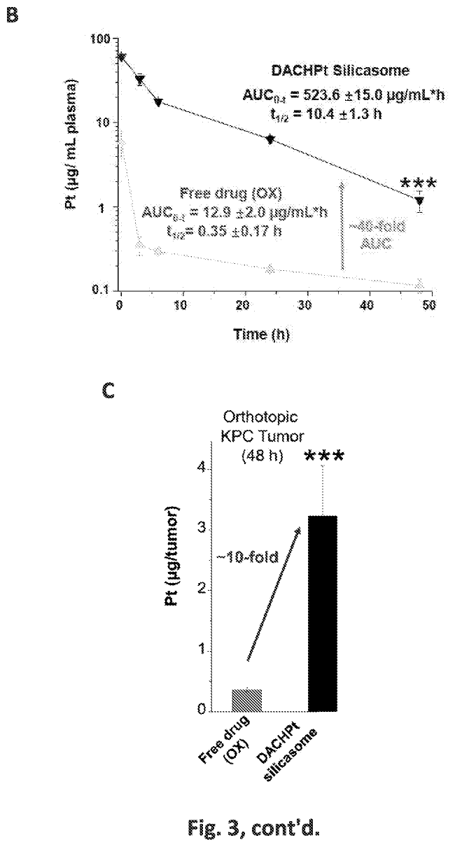

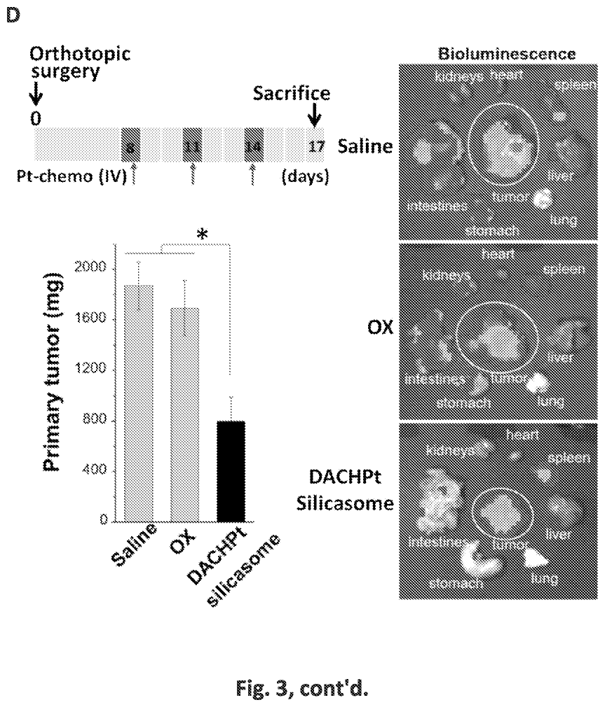

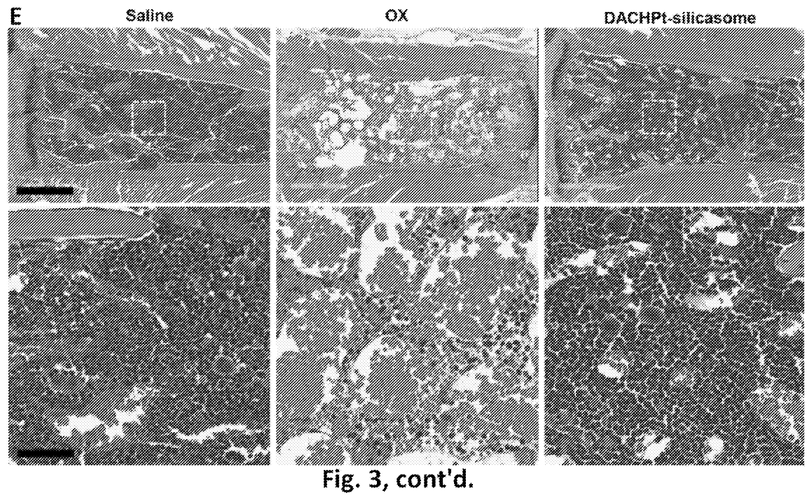

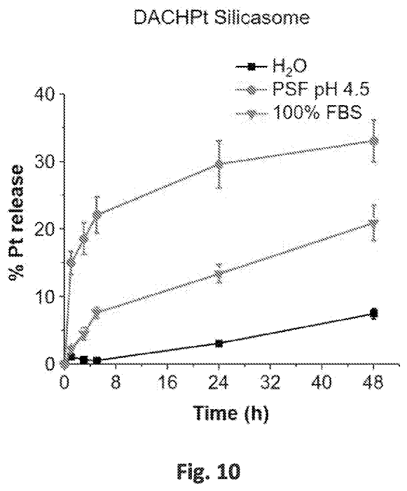

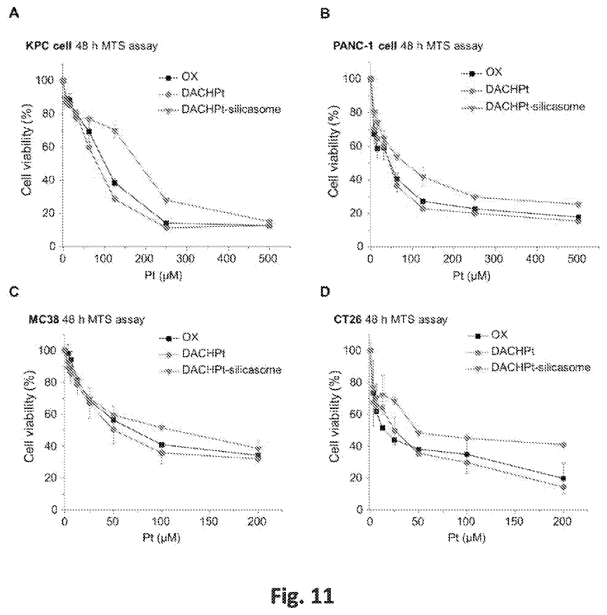

In order to investigate the in vivo relevance of improved DACHPt delivery by silicasomes in an animal tumor model, a freshly prepared batch of DACHPt silicasomes were prepared and characterized, as shown in FIG. 3, panel A. This includes assessment of the primary and hydrodynamic particle sizes by cryoEM and DLS, which yielded a particle sizes of 82.4±2.8 nm and 137.3±1.1 nm (PDI 0.076), respectively. High-magnification cryoEM images confirmed that there was uniform coating on the MSNP surface by an intact 6-7 nm thick lipid bilayer (FIG. 3, panel A). The particles were also used to determine drug release by incubation in H₂O, acidified phagolysosomal simulation fluid (PSF) buffer (pH 4.5) and 100% serum (FIG. 10). This showed ˜6% drug release from the carrier in H₂O at 48 h, compared to ˜20% and ˜35% by serum and PSF buffer, respectively (FIG. 10). Before conducting animal experiment, we also assessed the impact of the carriers on the viability of human PANC-1 cells as well as murine KPC cells. The latter cell line was derived from a spontaneous Kras^{LSL-G12D/+}; Trp53^{LSL-R172H/+}; Pdx-1-Cre PDAC tumor^{[20]}. A MTS assay was used to demonstrate that the DACHPt silicasome resulted in roughly the same decrease in cell viability, as the free drug over 48 h (FIG. 11, panels A and B). Since oxaliplatin is also useful for the treatment of colon cancer, similar cell viability experiments were carried out in MC38 and CT26 colon cancer cells. This demonstrated a dose-dependent cytotoxic effect (FIG. 11, panels C and D).

To further investigate the impact of the silicasome carrier in an orthotopic PDAC model, KPC cells were surgically implanted into the tail of the pancreas to establish a primary cancer that develops metastatic spread and resembles human PDAC in the expression of a robust dysplastic stroma and poor anti-PDAC immunity^{[20]}. For ease of tumor visualization, the KPC cells were stably transfected with a luciferase vector, as previously described^{[4a, 21]} In order to assess the pharmacokinetics (PK) of the DACHPt silicasome, the plasma Pt content was quantitatively assessed by ICP-MS in animals receiving a single IV injection of 50 mg/kg MSNPs that contain Pt drug equal to 10 mg/kg oxaliplatin. IV injection of non-encapsulated oxaliplatin (at identical Pt molar dose) served as the free drug control because free DACHPt leads to drug precipitates when interacting with Cl ions in the blood^{[7b]}. Blood collection was performed at 5 mins, 3 h, 6 h, 24 h and 48 h after IV injection, followed by ICP-MS quantification. Circulatory half-life (t_{1/2}) was calculated to be 10.4±1.3 h and 0.35±0.17 h for the silicasome vs. the free drug, respectively, in making use of a one-compartment model^{[4]} (FIG. 3, panel B). The area-under-the-curve (AUC) for the encapsulated DACHPt was determined to be 523.6±15.0 μg/mL·h, which was 40-fold higher than that of free drug (labeled as “OX”). To assess Pt drug content in tumor, orthotopic KPC tumor-bearing mice received a single IV injection of drug-laden particles as in the PK study, followed by sacrifice at 48 h. Intratumoral drug content improved ˜10-fold for encapsulated vs. free drug delivery (FIG. 3, panel C), which is highly significant (p<0.001). The DACHPt silicasome also showed abundant distribution to the liver and spleen, while the kidney also showed a relatively high Pt level for the encapsulated vs. the free drug (FIG. 12).

An efficacy study was performed by IV injection of the silicasome carrier eight days after orthotopic KPC implantation in the pancreas of B6129SF1/J mice (FIG. 3, panel D). This corresponds to beginning the treatment at a primary tumor size of 5-10 mm in the absence of macro-metastases^{[21]}. IV injections were repeated every 3 days for a total of 3 repeats. The control groups included animals receiving IV saline or free oxaliplatin. The tumor growth at the primary and metastatic sites was followed by IVIS bioluminescence imaging up to conclusion of the experiment on day 17 (FIG. 3, panel D, right). This demonstrated a lower tumor burden and metastatic spread in response to treatment with the DACHPt silicasome compared to saline and free drug (p<0.05). We also assessed the primary tumor weight, which demonstrated ˜60% tumor inhibition by the DACHPt-loaded silicasome compared to ˜5% inhibition by the free drug (FIG. 3, panel D, left). This difference was statistically significant (p<0.05).

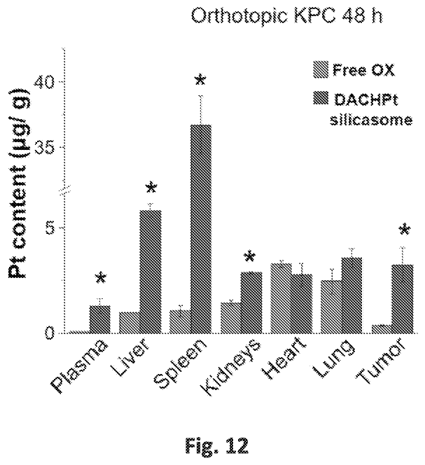

Overcoming chemotherapy side effects is an important objective of nano-based chemo drug delivery^{[22]}. In order to determine if treatment safety also applies to the DACHPt silicasome, histological analysis was performed on all the main organs and the bone marrow. In contrast to the major (>50%) reduction in cellularity of the hemopoetic cells by the free drug in the bone marrow, there was no noticeable hypocellularity during treatment with the DACHPt silicasome, similar to the effect of saline administration (FIG. 3, panel E). This finding could be of major significance to PDAC patients receiving oxaliplatin therapy and who frequently develop grade 3-4 neutropenia^{[5]}. Histological examination of other major organs, such as liver, spleen, kidney, lung and heart, did not show any gross pathology in any of the treatment groups (FIG. 13).

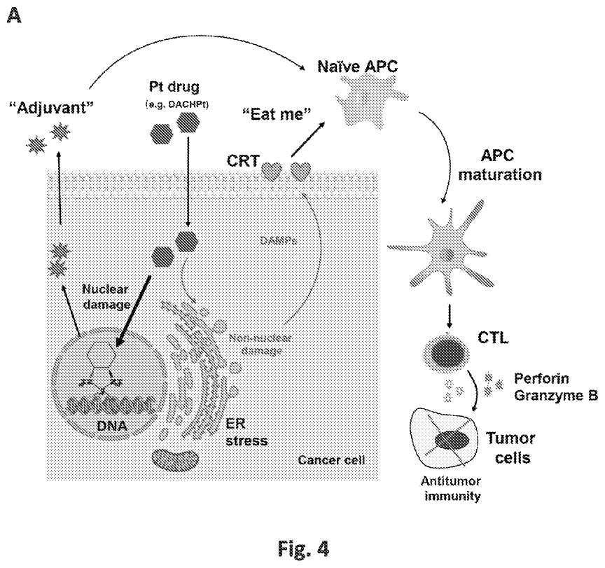

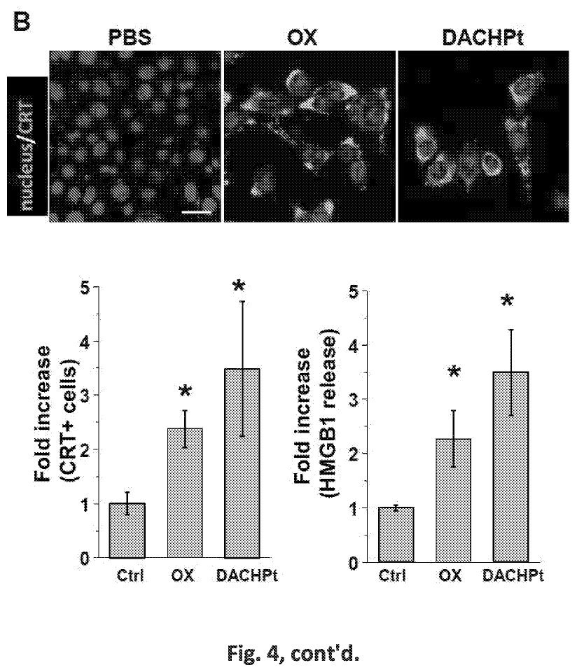

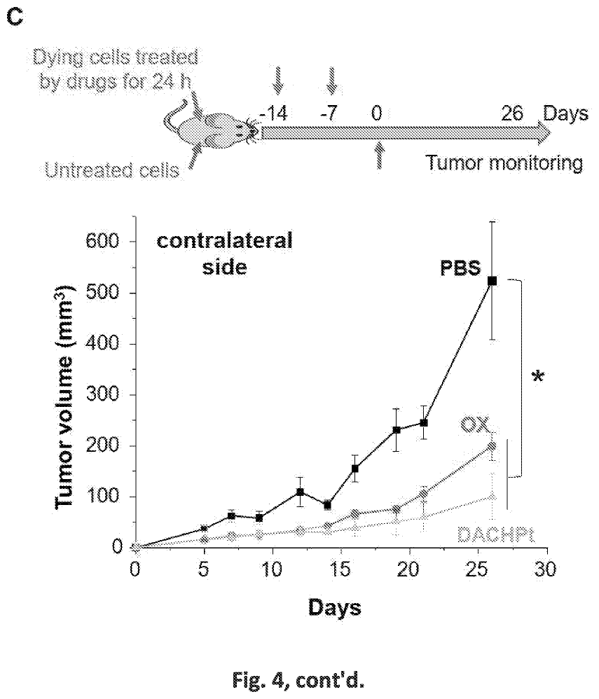

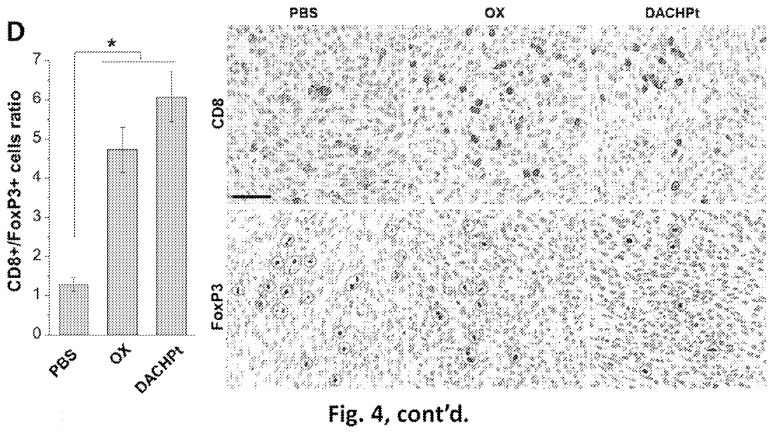

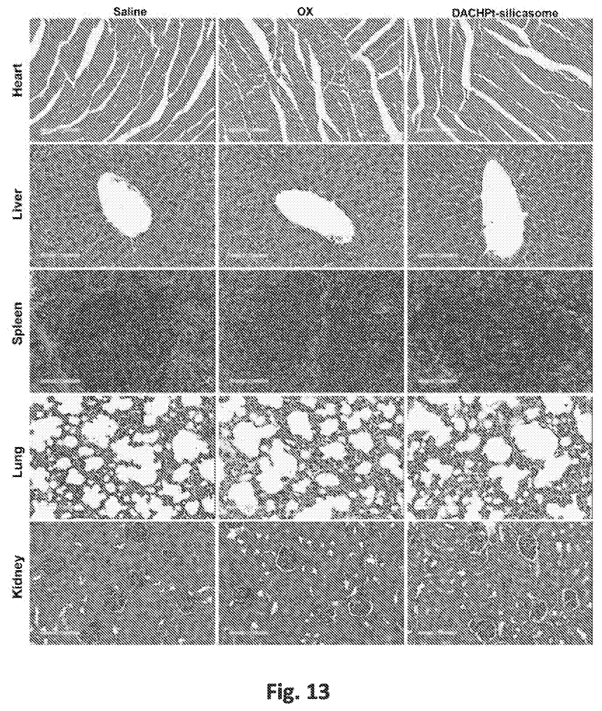

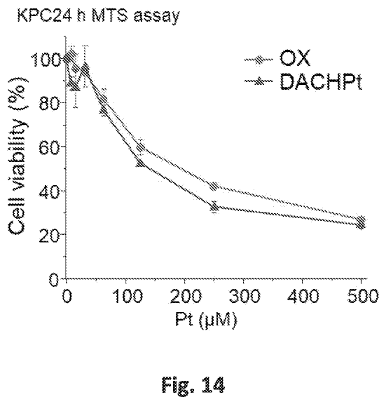

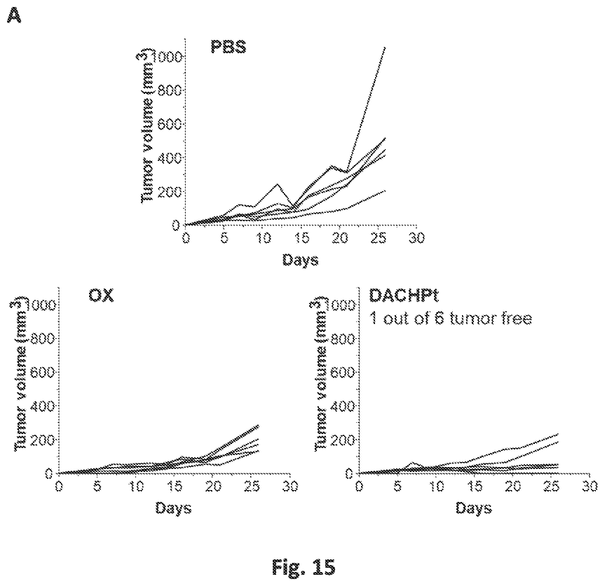

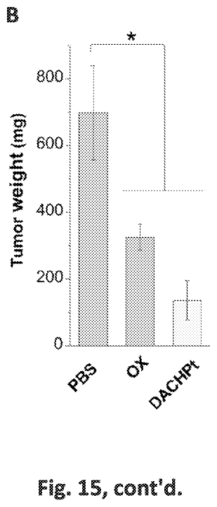

While clearly efficacious for chemotherapy delivery in the orthotopic PDAC model, we were also interested to see whether encapsulated DACHPt exerts an immunogenic cell death (ICD) effect, similar to what was described for oxaliplatin^{[23]}. ICD represents a unique form of apoptotic cell death that is accompanied by a chemo-induced cell stress response that is characterized by the expression of calreticulin (CRT) and the release of adjuvant stimuli^{[23a, 24]}. While CRT expression on the dying tumor cell surface provides an “eat-me” signal to antigen-presenting cells (APC)^{[23]}, the subsequent release of high-mobility group box 1 (HMGB1) from the dying tumor cells provide an adjuvant stimulus for APC maturation (FIG. 4, panel 4A)^{[23a, 23c-f, 25]}. Ample evidence also suggest that it is possible to combine an ICD stimulus with immune checkpoint inhibitors (ICI) such as anti-PD-1 for combination immunotherapy^{[25]}. In order to determine whether DACHPt induces CRT expression and HMGB1 release in KPC cells, an in vitro experiment was performed in cultured cells that were exposed to the DACHPt as well as oxaliplatin (FIG. 14). This demonstrated effective killing of 70% of the cells by both treatments after 24 h. Immunofluorescence staining with an anti-CRT primary antibody, followed by Alexa Fluor® 488 conjugated secondary antibody, demonstrated a significant increase cell surface fluorescence of CRT, as determined by flow cytometry (FIG. 4, panel B, upper panel). We also performed an ELISA assay to measure HMGB1 release into the supernatant, which demonstrated increase of released HMGB1 from treated cells (FIG. 4, panel B, lower panel). In order to determine whether these in vitro ICD markers actually represent an in vivo immunogenic effect as specified by a consensus ICD document^{[26]}, we also performed a vaccination study in a syngeneic KPC animal model. Suspensions of dying tumor cells, generated by oxaliplatin or DACHPt treatment for 24 h, were subcutaneously injected on 2 occasions (7 days apart) in left flank of immunocompetent B6129SF1/J mice. This was followed by tumor re-challenge on the contralateral side using untreated KPC cells (FIG. 4, panel C). The magnitude of the growth inhibition on the contralateral side demonstrated growth retardation for both treatments, implying immunogenic effects (p<0.05). Notably, 1 (out of 6) mouse in the DACHPt-treated group, remained tumor free (see “spaghetti curves” in FIG. 15, panel A). Tumor weight measurement (FIG. 15, panel B) confirmed the reduction in tumor volume. Importantly, immunohistochemistry (IHC) analysis of the remove tumor tissue showed an increase in infiltration by CD8⁺ T-cells in parallel with a reduced number of Foxp3⁺ regulatory T cell (Treg) for both oxaliplatin and DACHPt treatment (FIG. 4, panel D). This amounted to a respective increase of 4.8- and 6-fold in the CD8⁺/Tregs cell ratios compared to PBS control (FIG. 4, panel D).

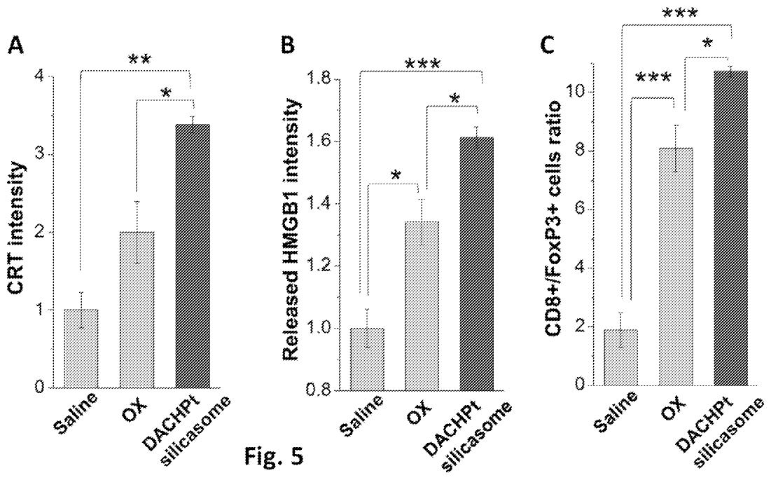

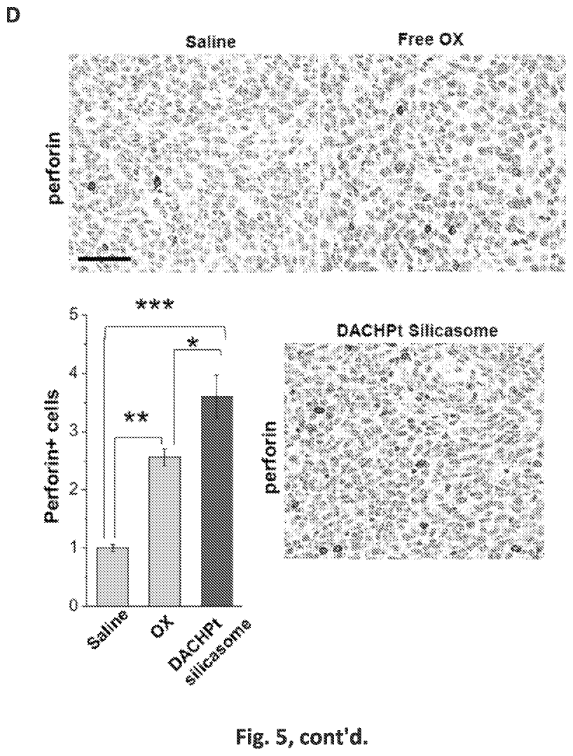

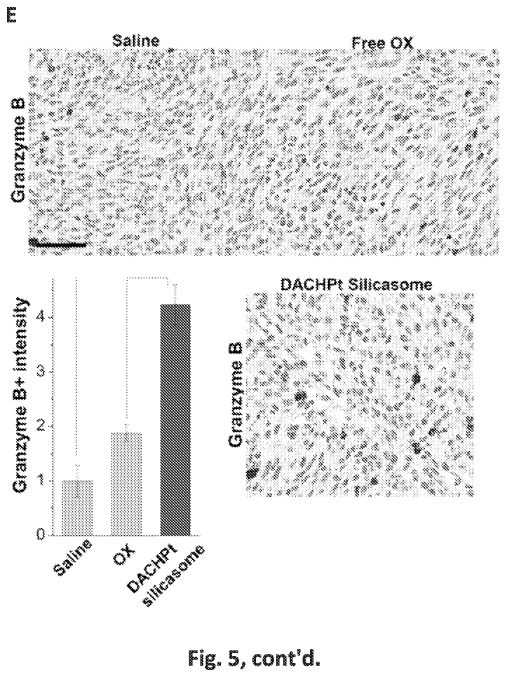

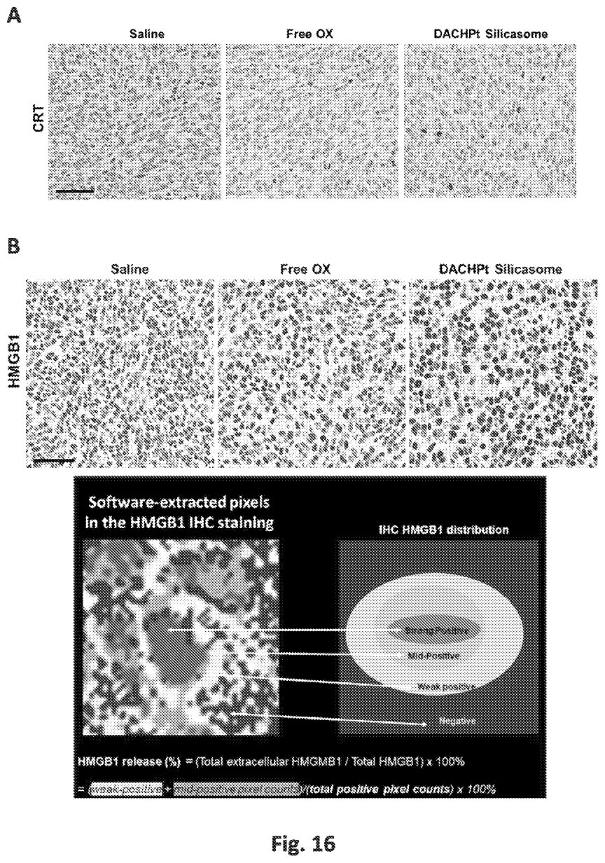

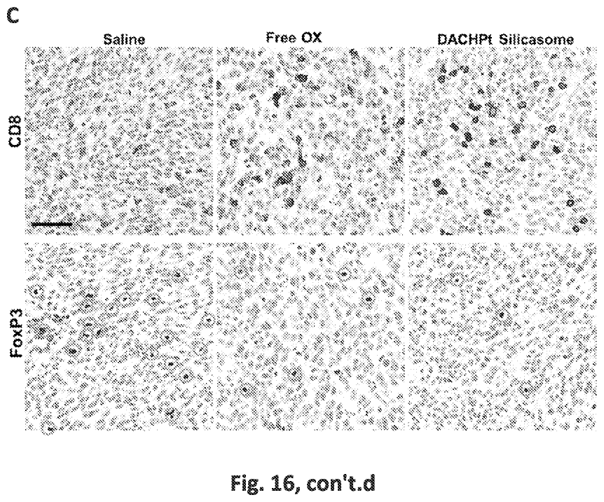

Given this background, we proceeded to the performance of IHC staining for ICD markers, and immune activation in the tumor tissues harvested from the animals treated with the DACHPt silicasomes and oxaliplatin in FIG. 3, panel D. This demonstrated that encapsulated DACHPt was capable of inducing significantly higher CRT expression compared to animals treated with free drug (p<0.05), or saline (p<0.01) (FIG. 5, panel A). Representative IHC pictures for CRT staining appeared online (FIG. 16, panel A). A similar finding was made during performance of IHC staining for HMGB1 release (FIG. 5, panel B); this required additional software analysis to quantify the release of the protein from damaged nuclei (FIG. 16, panel B). Similar to the vaccination study, we also evaluated the number of CD8⁺ T cells and FoxP3⁺ Tregs at the tumor site. This resulted in a significant increase in the CD8⁺/Treg ratio compared to free drug (p<0.05) or the saline control (p<0.001) (FIG. 5, panel C). Representative IHC images appear online (FIG. 16). Moreover, we observed significantly increased staining for perforin and granzyme B at the tumor site of animals treated with the DACHPt silicasome compared to free drug (p<0.01) or the saline control (p<0.001) (FIG. 5, panels D and E). Representative IHC images appear on the right side of each panel.

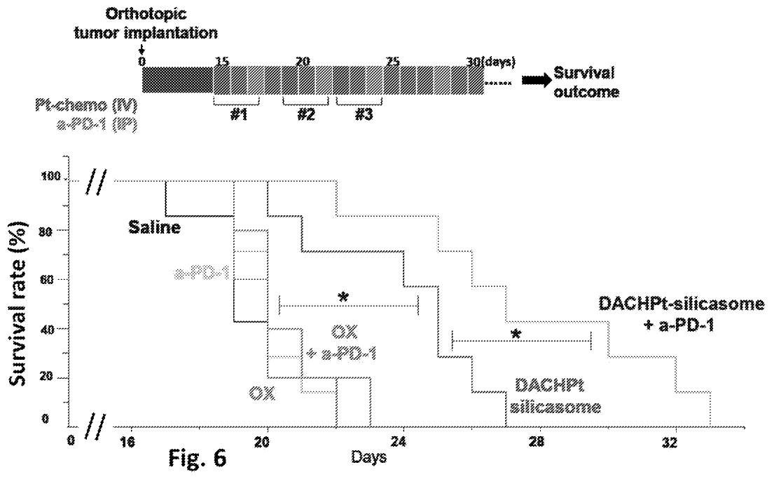

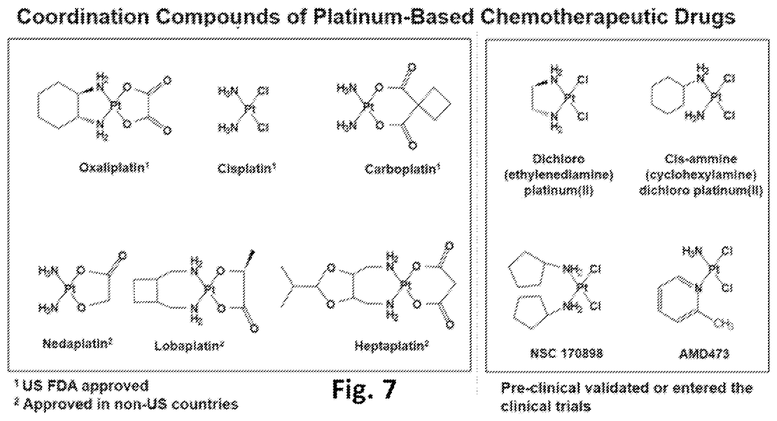

To confirm the therapeutic benefit of the DACHPt silicasome in the efficacy study, we also performed a survival outcome study in the KPC orthotopic model, using the same dosimetry, and frequency of DACHPt silicasome administration as for FIG. 3, panel D, with minor modifications (FIG. 6). We also included the use of the treatment with anti-PD-1 in this analysis to determine whether the generation of a “hot” immune environment (as a result of the ICD effect by encapsulated DACHPt) could affect the survival outcome through a chemo-immunotherapy effect. Consistent with the efficacy study, the DACHPt silicasome significantly improved the survival outcome compared to saline and free oxaliplatin in the syngeneic orthotopic KPC model (p<0.05). Moreover, the performance of the already efficacious DACHPt silicasome was further enhanced by anti-PD-1 antibody, leading to a significant prolongation of the animal life span (p<0.05). No obvious benefit was observed in the treatment using free oxaliplatin alone or anti-PD-1 alone. This likely reflects inadequate oxaliplatin access to the tumor site^{[27]}, as well as low immunogenicity of the PDAC tumor as a result of the low mutational load and a relatively “cold” tumor microenvironment in the absence of an immunogenic stimulus^{[28]}.

### Conclusions

In this study, we developed a facile and effective drug loading approach to allow Pt drug delivery by a silicasome nanocarrier. This required the use of activated Pt drugs for efficient loading, and electrostatic/coordination attachment to the porous interior under weak basic conditions. Additional coating of the MSNPs surface by a lipid bilayer allowed secured entrapment of the Pt drug molecules led to providing colloidal stability and successful systemic biodistribution. This was demonstrated by the improved PK profile and intratumoral drug delivery by the DACHPt laden silicasome over free drug in an orthotopic KPC model. IV injection of DACHPt silicasome also demonstrated efficacy in the chemotherapy response of the tumor along with a significant reduction in bone marrow toxicity. In addition, the tumor killing response was associated with an immunogenic cell death response that was reflected by increased biomarker for ICD and cytotoxic T-cell generated tumor cell death. In a separate survival experiment, IV injection of DACHPt silicasome led to a significantly improved survival benefit compared to the free drug. Moreover, the efficacy of the chemo-immunotherapy response was further enhanced by the co-administration of anti-PD-1 antibody. Collectively, the successful development of a facile and versatile Pt silicasome offers great promise in improving the therapeutic index of Pt-based chemotherapy agents, both as a monotherapy as well as for combination therapy with the new checkpoint inhibitors, based on the additional dimension of an immunogenic effect of the delivered drugs.

### Experimental

Materials

Tetraethylorthosicate (TEOS), triethanolamine (TEA-ol), triethylamine (TEA) cetyltrimethylammonium chloride solution (CTAC, 25 wt % in water), silver nitrate(AgNO₃), nitric acid (HNO₃), 4-(2-Hydroxyethyl)piperazine-1-ethanesulfonic acid (HEPES), dextrose, dichloro(1,2-diaminocyclohexane)platinum(II) (DACHPtCl₂), cis-diammineplatinum(II) dichloride (cisplatin), and dichloro(ethylenediamine)platinum(II) (Pt(en)Cl₂) were purchased from Sigma-Aldrich, USA. 1,2-Distearoyl-sn-glycero-3-phosphocholine (DSPC), 1,2-distearoyl-sn-glycero-3-phospho-ethanol amine-N-[methoxy(polyethylene glycol)-2000] (ammonium salt) (DSPE-PEG₂₀₀₀), and cholesterol (Chol) were purchased from Avanti Polar Lipids, USA. Oxaliplatin was purchased from LC Laboratories, USA. Murine anti-PD-1 antibody (#BE0146) and InVivoPure pH 7.0 dilution buffer (#IP0070) were purchased Bio X Cell (New Hampshire, USA). Penicillin, streptomycin, Dulbecco's modified Eagle medium (DMEM) and Roswell Park Memorial Institute (RPMI) 1640 Medium were purchased from Invitrogen. Fetal bovine serum (FBS) was purchased from Gemini Bio Products. Matrigel™ Matrix Basement Membrane was purchased from BD Bioscience.

Preparation of Cationic and Activated Pt Drugs: DACHPt, DAPt and EDAPt

To achieve efficient drug loading, a list of cationic and activated Pt drugs were prepared as detailed in the literature, with minor modification^{[29]}. Taking DACHPt for example, DACHPtCl₂ (506 mg, 1.33 mmol) and AgNO₃ (406 mg, 2.39 mmol) were added in 9.37 mL DI H₂O. The molar ratio of AgNO₃:DACHPtCl₂ was 1.8:1. Subsequently, 93.7 μL of a 5% HNO₃ solution was added to the mixture to achieve an acidic pH of <2. The suspension was wrapped in aluminum foil and placed in a 70° C. oil bath, with stirring overnight (˜16 h). The mixture was cooled on ice and stored in a 4° C. refrigerator overnight. The sample was spun down at 4,000 rpm for 10 min and the supernatant filtrated through a 0.22 μm syringe filter to obtain the final product, DACHPt. The DAPt and EDAPt aqueous complexes were prepared in similar fashion from commercially available cisplatin and Pt(en)Cl₂, respectively. The ratio of AgNO₃:cisplatin (or Pt(en)Cl₂) remained the same (1.8:1), corresponding to 51 mg AgNO₃ plus 50 mg cisplatin or 47 mg AgNO₃ plus 50 mg Pt(en)Cl₂, respectively. The Pt concentration was determined by ICP-MS (NexION 2000, PerkinElmer). We also used ICP-MS to measure the Ag ion concentration to ensure the removal of AgCl from our samples.

Synthesis, Purification, and Characterization of Cationic and Activated Pt Drugs Laden Silicasomes

Sixty-five nm bare MSNPs were synthesized at 18 L scale and purified by extensive acidic ethanol washing to remove the CTAC detergent, as reported previously^{[4b]}. In order to determine the optimal loading condition, we experimented with multiple rounds of drug loading to find the optimal pH, incubation time, sonication condition, and particle/drug feed ratio, etc. Details of the protocol optimization appear online (FIG. 8). In the case of DACHPt loading, purified MSNP (20 mg) from ethanol stock solution was spun at 15,000 rpm for 15 min, and resuspend in 1 mL HEPES buffer (200 mM, pH 8.5). The MSNP suspension was spun down and resuspend in 0.5 mL HEPES buffer (200 mM, pH 8.5). Then, 160 μL DACHPt solution (25 mg/mL, e.q. Pt) and 340 μL pure H₂O were added to the MNSP suspension (40 mg/mL, 0.5 mL). The mixture was sonicated for 10 min. In a sonication water bath. The DACHPt-laden MSNPs were spun down at 15,000 rpm for 12 min, followed by collecting the supernatant for Pt elemental analysis by ICP-MS. This allowed us to calculate the amount of drug associated with MSNP. Drug-soaked MSNPs were further used to introduce surface lipid coating. Briefly, the DACHPt-laden MSNP pellet was resuspended in 1 mL 5% Dextrose 5 mM HEPES buffer (pH 7.4, absence of Cl⁻ ion) and added to the lipid suspended in an ethanol solution (32 mg DSPC, 10.8 mg Chol and 5.6 mg PE-PEG_{2K}, in 100 μL ethanol) at 65° C. The mixture was sonicated by probe sonication (Ultrasonic Processor Model VCX130, 40% amplitude) at a 10 s/5 s on/off cycle for 5˜10 mins. After sonication, the particles were purified by washing with 5% Dextrose 5 mM HEPES buffer (pH 7.4) through two rounds of centrifugation at 15,000 rpm for 15 mins. The washed DACHPt silicasome sample was re-suspended in 5% Dextrose 5 mM HEPES buffer (pH 7.4) and filtered across a 0.22 μm filter for sterilization. Similarly, the DAPt and EDAPt silicasomes were prepared by the similar procedure as above. In order to compare the complexation synthesis of Pt silicasomes to silicasomes that passively encapsulate oxaliplatin, cisplatin and Pt(en)Cl₂, 20 mg bare MSNPs were mixed with a 4 mg equivalent Pt of the drugs dissolved in DI water during vigorously stirring for 24 h. The drug soaked MSNPs were subsequently used for applying the lipid coating, as described above.

The Pt drug content of the final synthesized products was determined by ICP-OES or ICP-MS by diluting the sample in 2% HNO₃. The encapsulation efficiency was defined as EE %=[the total amount of encapsulated Pt drug (m₁)]/[the total amount of Pt drug (m₀)]×100%. Drug loading capacity was defined as LC %=[the total amount of encapsulated Pt drug (m_{drug})]/[the total amount of particle (m_{MSNP})]×100%. Particle hydrodynamic size and zeta potential were measured by a ZETAPALS instrument (Brookhaven Instruments Corporation). The final product was visualized by cryoEM (TF20 FEI Tecnai-G2) to confirm the uniformity and integrity of the coated lipid bilayer. The energy-dispersive X-ray spectroscopy (EDS) and element mapping were performed by scanning transmission electron microscopy (STEM) in a FEI Titan 80-300 kV TEM.

Cell Culture

The KPC pancreatic adenocarcinoma cell line, which was derived from a spontaneous tumor originating in a transgenic Kras^{LSL-G12D/+}; Trp53^{LSL-R172H/+}; Pdx-1-Cre mouse (B6/129 background)^{[4, 20b, 21]}, was cultured in DMEM, containing 10% FBS, 100 U/mL penicillin, 100 μg/mL streptomycin, 2 mM L-glutamine and 1 mM sodium pyruvate. To allow bioluminescence imaging, the KPC cells were permanently transfected with a luciferase-based lentiviral vector in the UCLA vector core facility, followed by a limiting dilution cloning as we previously described^{[4a]}.

Cytotoxicity MTS Assay

Cytotoxicity testing of free Pt drugs or drug-laden silicasomes was performed by using a standard MTS assay (CellTiter 96© AQueous One Solution Cell Proliferation Assay, Promega). PDAC cells were plated at a density of 5×10³ cells per well in a 96-well plate and cultured for 24 h before the medium was replaced with fresh medium containing free OX, free DACHPt or DACHPt laden silicasome at indicated concentrations. Non-treated cells were used as control. After treatment for 48 h, the medium was replaced with 100 μL of fresh medium containing MTS solution (5:1, v/v medium/CellTiter 96© Aqueous stock solution), and the cells were further cultured at 37° C. for 1 h. The absorbance of the culture wells at 490 nm was recorded by a microplate reader (M5e, Molecular Device, USA). Wells receiving the MTS solution without cells were used as blank. The relative cell viability (%) is [(the absorption of treated well−blank)/(the absorption of control well−blank)]×100%.

Animal Purchase and Study Permission

Female B6129SF1/J mice (JAX 101043) were purchased from The Jackson Laboratory, and maintained under pathogen-free conditions. All animal experiments were performed according to protocols (#2009-134) approved by the UCLA Animal Research Committee.

PK Study

The PK study was performed on 10-12-week-old healthy female B6129SF1/J mice. The animals received a single IV injection of free OX or DACHPt silicasome at a Pt dose of 4.95 mg/kg (equal to oxaliplatin dose of 10 mg/kg), followed by collection of blood samples at 5 min, 3, 6, 24, and 48 hrs. After separation of the plasma fraction, the plasma samples were digested with HCl:HNO₃ 3:1, v/v) in a hot-block, before replenishment in 2% HNO₃ for ICP-MS analysis of the Pt content. The PK data were analyzed by PKSolver software, using a one-compartment model^{[4]}.

Tumor Drug Content and Biodistribution Study

An orthotopic KPC tumor model in immunocompetent B6129SF1/J mouse was established as described previously^{[4, 21]}. Briefly, 30 μL of DMEM/Matrigel (1:1 v/v), containing ˜1×10⁶ KPC-luc cells, was injected into the tail of the pancreas in female B6129SF1/J mice (8˜10 weeks) by a sort surgical survival procedure^{[4, 21]}. To determine the tumor drug content and biodistribution, tumor bearing mice received a single IV injection of free OX or DACHPt silicasome at a Pt dose of 4.95 mg/kg. Animals were sacrificed 48 h post-injection, followed by tumor and tissue collections. These samples were accurately weighed, and followed by digestion using aqua regia in a hot-block and reconstructed in 2% HNO₃ for ICP-MS measurement to determine the Pt content.

Assessment of Anti-PDAC Efficacy by the DACHPt Silicasome in the Orthotopic KPC Model Described Above

KPC-luc cells (˜1×10⁶) were orthotopically injected into the pancreas in mice. Eight-day post-surgery, the tumor-bearing mice received IV injections of DACHPt silicasome at Pt dose of 2 mg/kg. The control includes saline as well as free oxaliplatin. This dose arrangement is in agreement with the literature^{[27]}. Tumor-bearing mice received IV injection of the indicated therapy every 3 days for a total of 3 administrations. Before animal sacrifice (72 h post the last IV injection), the mice received intraperitoneal injection of D-luciferin, followed by ex vivo bioluminescence imaging using an IVIS imaging system. Primary tumor and major organs (e.g. sternum, heart, liver, spleen, lung and kidneys) were harvested and fixed in 10% formalin, followed by paraffin embedding and sectioning to provide 4 μm slices for histological analysis in the UCLA Translational Pathology Core Laboratory (TPCL). H&E staining was performed to look at the pathological abnormality in mice receiving different treatments. The H&E slides for toxicity assessment were read in a blinded fashion by an experienced veterinary pathologist.

Identification of DACHPt as an ICD-Inducing Agent

Surface CRT expression was visualized by immunofluorescence (IF) staining using an anti-CRT primary antibody, followed by the incubation with Alex488 conjugated secondary antibody. Briefly, ˜1.5×10⁴ KPC cells were seeded into an 8-well confocal chamber slide. After 24 h, the cell culture medium was replaced with fresh medium containing the chemo agents, following which the cells were incubated for another 24 h. The cells were washed twice in cold PBS and fixed with 4% paraformaldehyde (PFA) at room temperature (r.t.) for 15 mins. After fixation, the cells were washed twice with cold PBS and blocked with 1% BSA in PBS for 0.5 h. The cells were incubated with anti-CRT primary antibody (ab2907, 1:200) in 200 μL blocking solution at 4° C. overnight, followed by washing with PBS and staining with secondary antibody (Alex488 conjugated goat anti-rabbit secondary antibody, A-11008, 1:1000) together with the nuclear dye, Hoechst 33342, at r.t. for 1 h. The cells were washed with PBS, then imaged by using a Leica SP8-MD confocal microscope under the 100× objective lens.

Surface CRT expression was measured by flow cytometry as previously described^{[24a]}. Briefly, 7.5×10⁴ KPC cells were seeded into 24-well plates. After cell attachment, KPC cells were treated with free oxaliplatin and DACHPt (500 μM), for 24 h. The loosely attached cells were combined with trypsin-treated at adherent cells. The cells were washed in cold PBS and then stained on ice with a primary anti-CRT antibody (Abcam, ab2907, 1:140) in 200 μL BD staining buffer for 0.5 h. The cells were washed in cold PBS and stained with an Alexa Fluor® 680-conjugated secondary antibody (LifeScience Technologies #A21244) for 30 min on ice. After washing in cold PBS, the cells were assessed in a LSRII flow cytometer (BD Biosciences). In the same experiment, the cell culture media were spun down to collect the supernatants for HMGB1 detection by an ELISA kit (Catalog #ST51011, IBL International GmbH), according to the manufacture's instruction.

Moreover, we also validated the ICD effect of DACHPt in a vaccination experiment, using a published protocol^{[26]}. Briefly, eight million KPC cells were seeded in a tissue culture dish. After cellular attachment, DACHPt or free oxaliplatin (500 μM) were added for 24 h. Cells were collected and washed before being resuspended in 0.8 mL cold PBS. For vaccination, each mouse received subcutaneous injection (SC) of a 100 μL suspension of chemo-treated cells in the right flank. Control animals received PBS only. The vaccination was repeated after 7 days. Fourteen days after the 1^{st} vaccination, the same mice received the SC injection of healthy KPC cells (1×10⁶ cells) in the contralateral side. Tumor growth was measured by a digital caliper every 2-3 days. At the conclusion of the vaccination experiment (Day 26), animals were sacrificed and the tumors collected for IHC immunophenotyping of CD8⁺ T cells and FoxP3⁺ Treg cells. Primary antibodies to CD8 (#14-0808-82, 1:100) and FoxP3 (#13-5773-82, 1:200) were purchased from ThermoFisher. IHC staining was performed in the UCLA Translational Pathology Core Laboratory (TPCL). The slides were scanned and images were assessed by using Aperio ImageScope software (Leica).

Investigation of ICD and Immune Activation by Silicasome Encapsulated DACHPt in the Orthotopic KPC Model

The tumor tissues in the efficacy study (FIG. 3, panel D) were used for further immunophenotyping with the focus of ICD induction and immune activation. Primary antibodies that recognizes ICD biomarkers include CRT (ab2907, 1:200) and HMGB1 (ab18256, 1:200). We also measured immune activation markers, such as CD8⁺ T cells (#14-0808-82, 1:100), FoxP3⁺ Treg (#13-5773-82, 1:200), granzyme B (ab4059, 1: 100), and perforin (ab16074, 1:100) through IHC staining. The slides were scanned and images were analyzed by using Aperio ImageScope software (Leica).

Assessment of the Survival Outcome Using DACHPt Silicasome w/Wo Anti-PD-1 in an Orthotopic KPC Tumor Model

Tumor-bearing mice were randomly assigned into 6 groups (n=5-7) and received IV injection of Pt drug formulations twice per week as designed in FIG. 6. Anti-PD-1 antibody was injected at 100 μg/animal intraperitoneally two days later after each Pt chemo injection. To assess survival rate, animals were monitored daily up to the stage of spontaneous death or approaching moribund status based on the criteria of extensive abdominal ascites, severe dehydration, significant weight loss (>20%), extreme weakness or inactivity^{[4, 21, 30]} The survival data were plotted as Kaplan-Meier curves, followed by statistical analysis by Log Rank testing (Mantel-Cox), using GraphPad Prism 7.00 software.

Statistical Analysis

Comparative analysis of differences between groups was performed using the 2-tailed Student's t-test (Excel software, Microsoft) for two-group comparison. One-way ANOVA followed by a Tukey's test (Origin software, OriginLab) was performed for multiple group comparisons. Data were expressed as mean±SD or SEM, as stated in the figure legends. The survival analysis was performed by Log Rank testing (Mantel-Cox), using GraphPad Prism 7.00 software. A statistically significant difference was considered at *p<0.05.

### REFERENCES

[1] R. L. Siegel, K. D. Miller, A. Jemal, CA Cancer J Clin 2019, 69, 7.
[2] a) I. Garrido-Laguna, M. Hidalgo, Nat Rev Clin Oncol 2015, 12, 319; b) J. P. Neoptolemos, J. Kleeff, P. Michl, E. Costello, W. Greenhalf, D. H. Palmer, Nature reviews. Gastroenterology & hepatology 2018, 15, 333.
[3] a) T. Conroy, F. Desseigne, M. Ychou, O. Bouche, R. Guimbaud, Y. Becouarn, A. Adenis, J. L. Raoul, S. Gourgou-Bourgade, C. de la Fouchardiere, J. Bennouna, J. B. Bachet, F. Khemissa-Akouz, D. Pere-Verge, C. Delbaldo, E. Assenat, B. Chauffert, P. Michel, C. Montoto-Grillot, M. Ducreux, U. Groupe Tumeurs Digestives of, P. Intergroup, N Engl J Med 2011, 364, 1817; b) M. Suker, B. R. Beumer, E. Sadot, L. Marthey, J. E. Faris, E. A. Mellon, B. F. El-Rayes, A. Wang-Gillam, J. Lacy, P. J. Hosein, S. Y. Moorcraft, T. Conroy, F. Hohla, P. Allen, J. Taieb, T. S. Hong, R. Shridhar, I. Chau, C. H. van Eijck, B. G. Koerkamp, The Lancet. Oncology 2016, 17, 801; c) T. Conroy, P. Hammel, M. Hebbar, M. Ben Abdelghani, A. C. Wei, J. L. Raoul, L. Chone, E. Francois, P. Artru, J. J. Biagi, T. Lecomte, E. Assenat, R. Faroux, M. Ychou, J. Volet, A. Sauvanet, G. Breysacher, F. Di Fiore, C. Cripps, P. Kavan, P. Texereau, K. Bouhier-Leporrier, F. Khemissa-Akouz, J. L. Legoux, B. Juzyna, S. Gourgou, C. J. O'Callaghan, C. Jouffroy-Zeller, P. Rat, D. Malka, F. Castan, J. B. Bachet, G. Canadian Cancer Trials, G. I. P. G. the Unicancer, N Engl J Med 2018, 379, 2395.
[4] a) X. S. Liu, A. Situ, Y. A. Kang, K. R. Villabroza, Y. P. Liao, C. H. Chang, T. Donahue, A. E. Nel, H. Meng, Acs Nano 2016, 10, 2702; b) X. Liu, J. Jiang, R. Chan, Y. Ji, J. Lu, Y. P. Liao, M. Okene, J. Lin, P. Lin, C. H. Chang, X. Wang, I. Tang, E. Zheng, W. Qiu, Z. A. Wainberg, A. E. Nel, H. Meng, ACS Nano 2019, 13, 38.
[5] a) J. Cassidy, S. Clarke, E. Diaz-Rubio, W. Scheithauer, A. Figer, R. Wong, S. Koski, M. Lichinitser, T. S. Yang, F. Rivera, F. Couture, F. Sirzeen, L. Saltz, Journal of Clinical Oncology 2008, 26, 2006; b) D. G. Haller, Oncology 2000, 14, 15.
[6] S. V. Hato, A. Khong, I. J. de Vries, W. J. Lesterhuis, Clin Cancer Res 2014, 20, 2831.
[7] a) M. Galanski, M. A. Jakupec, B. K. Keppler, Curr Med Chem 2005, 12, 2075; b) N. J. Wheate, S. Walker, G. E. Craig, R. Oun, Dalton Trans 2010, 39, 8113.
[8] H. S. Oberoi, N. V. Nukolova, A. V. Kabanov, T. K. Bronich, Adv Drug Deliv Rev 2013, 65, 1667.
[9] D. Wang, S. J. Lippard, Nat Rev Drug Discov 2005, 4, 307.
[10] a) T. C. Johnstone, K. Suntharalingam, S. J. Lippard, Chem Rev 2016, 116, 3436; b) P. A. Ma, H. H. Xiao, C. X. Li, Y. L. Dai, Z. Y. Cheng, Z. Y. Hou, J. Lin, Materials Today 2015, 18, 554; c) Z. Hang, M. A. Cooper, Z. M. Ziora, Biochemical Compounds 2016, 4, 1; d) H. H. Xiao, L. S. Yan, E. M. Dempsey, W. T. Song, R. G. Qi, W. L. Li, Y. B. Huang, X. B. Jing, D. F. Zhou, J. X. Ding, X. S. Chen, Progress in Polymer Science 2018, 87, 70; e) Y. Mochida, H. Cabral, K. Kataoka, Expert Opin Drug Deliv 2017, 14, 1423.
[11] C. O. Arean, M. J. Vesga, J. B. Parra, M. R. Delgado, Ceramics International 2013, 39, 7407.
[12] J. Gu, S. Su, Y. Li, Q. He, J. Zhong, J. Shi, The Journal of Physical Chemistry Letters 2010, 1, 3446.
[13] C. H. Lin, S. H. Cheng, W. N. Liao, P. R. Wei, P. J. Sung, C. F. Weng, C. H. Lee, Int J Pharm 2012, 429, 138.
[14] H. He, H. Xiao, H. Kuang, Z. Xie, X. Chen, X. Jing, Y. Huang, Colloids and Surfaces B: Biointerfaces 2014, 117, 75.
[15] Y. Zheng, C. D. Fahrenholtz, C. L. Hackett, S. Ding, C. S. Day, R. Dhall, G. S. Marrs, M. D. Gross, R. Singh, U. Bierbach, Chemistry-A European Journal 2017, 23, 3386.
[16] a) N. Martinho, T. C. B. Santos, H. F. Florindo, L. C. Silva, Frontiers in Physiology 2019, 9, 1898; b) E. Raymond, S. Faivre, S. Chaney, J. Woynarowski, E. Cvitkovic, Molecular cancer therapeutics 2002, 1, 227.
[17] a) M. Salerno, D. Yahia, S. Dzamitika, E. de Vries, E. Pereira-Maia, A. Gamier-Suillerot, JBIC Journal of Biological Inorganic Chemistry 2009, 14, 123; b) M. Murakami, H. Cabral, Y. Matsumoto, S. Wu, M. R. Kano, T. Yamori, N. Nishiyama, K. Kataoka, Sci Transl Med 2011, 3, 64ra2.
[18] D. Tam, C. E. Ashley, M. Xue, E. C. Carnes, J. I. Zink, C. J. Brinker, Acc Chem Res 2013, 46, 792.
[19] H. Zhang, D. R. Dunphy, X. Jiang, H. Meng, B. Sun, D. Tam, M. Xue, X. Wang, S. Lin, Z. Ji, R. Li, F. L. Garcia, J. Yang, M. L. Kirk, T. Xia, J. I. Zink, A. Nel, C. J. Brinker, J Am Chem Soc 2012, 134, 15790.
[20] a) S. R. Hingorani, L. Wang, A. S. Multani, C. Combs, T. B. Deramaudt, R. H. Hruban, A. K. Rustgi, S. Chang, D. A. Tuveson, Cancer Cell 2005, 7, 469; b) W. W. Tseng, D. Winer, J. A. Kenkel, O. Choi, A. H. Shain, J. R. Pollack, R. French, A. M. Lowy, E. G. Engleman, Clin Cancer Res 2010, 16, 3684.
[21] X. Liu, P. Lin, I. Perrett, J. Lin, Y. P. Liao, C. H. Chang, J. Jiang, N. Wu, T. Donahue, Z. Wainberg, A. E. Nel, H. Meng, J Clin Invest 2017, 127, 2007.
[22] a) D. Peer, J. M. Karp, S. Hong, O. C. FaroKHzad, R. Margalit, R. Langer, Nature Nanotechnology 2007, 2, 751; b) X. Liu, I. Tang, Z. A. Wainberg, H. Meng, Small 2020, 2000673.
[23] a) A. D. Garg, D. Nowis, J. Golab, P. Vandenabeele, D. V. Krysko, P. Agostinis, Biochimica et biophysica acta 2010, 1805, 53; b) X. Zhao, K. Yang, R. Zhao, T. Ji, X. Wang, X. Yang, Y. Zhang, K. Cheng, S. Liu, J. Hao, H. Ren, K. W. Leong, G. Nie, Biomaterials 2016, 102, 187; c) J. Pol, E. Vacchelli, F. Aranda, F. Castoldi, A. Eggermont, I. Cremer, C. Sautes-Fridman, J. Fucikova, J. Galon, R. Spisek, E. Tartour, L. Zitvogel, G. Kroemer, L. Galluzzi, Oncoimmunology 2015, 4, e1008866; d) L. Galluzzi, A. Buque, O. Kepp, L. Zitvogel, G. Kroemer, Cancer Cell 2015, 28, 690; e) L. Bezu, L. C. Gomes-da-Silva, H. Dewitte, K. Breckpot, J. Fucikova, R. Spisek, L. Galluzzi, O. Kepp, G. Kroemer, Frontiers in immunology 2015, 6, 187; f) G. Kroemer, L. Galluzzi, O. Kepp, L. Zitvogel, Annu Rev Immunol 2013, 31, 51.
[24] a) M. Obeid, A. Tesniere, F. Ghiringhelli, G. M. Fimia, L. Apetoh, J. L. Perfettini, M. Castedo, G. Mignot, T. Panaretakis, N. Casares, D. Metivier, N. Larochette, P. van Endert, F. Ciccosanti, M. Piacentini, L. Zitvogel, G. Kroemer, Nat Med 2007, 13, 54; b) L. Zitvogel, O. Kepp, L. Senovilla, L. Menger, N. Chaput, G. Kroemer, Clin Cancer Res 2010, 16, 3100; c) D. V. Krysko, A. D. Garg, A. Kaczmarek, O. Krysko, P. Agostinis, P. Vandenabeele, Nature reviews. Cancer 2012, 12, 860.
[25] C. Pfirschke, C. Engblom, S. Rickelt, V. Cortez-Retamozo, C. Garris, F. Pucci, T. Yamazaki, V. Poirier-Colame, A. Newton, Y. Redouane, Y. J. Lin, G. Wojtkiewicz, Y. Iwamoto, M. Mino-Kenudson, T. G. Huynh, R. O. Hynes, G. J. Freeman, G. Kroemer, L. Zitvogel, R. Weissleder, M. J. Pittet, Immunity 2016, 44, 343.
[26] O. Kepp, L. Senovilla, I. Vitale, E. Vacchelli, S. Adjemian, P. Agostinis, L. Apetoh, F. Aranda, V. Barnaba, N. Bloy, Oncoimmunology 2014, 3, e955691.
[27] H. Cabral, M. Murakami, H. Hojo, Y. Terada, M. R. Kano, U. I. Chung, N. Nishiyama, K. Kataoka, Proc Natl Acad Sci USA 2013, 110, 11397.
[28] a) C. Feig, J. O. Jones, M. Kraman, R. J. Wells, A. Deonarine, D. S. Chan, C. M. Connell, E. W. Roberts, Q. Zhao, O. L. Caballero, Proceedings of the National Academy of Sciences 2013, 110, 20212; b) J. W. Lee, C. A. Komar, F. Bengsch, K. Graham, G. L. Beatty, Current protocols in pharmacology 2016, 73, 14.39. 1; c) R. Winograd, K. T. Byrne, R. A. Evans, P. M. Odorizzi, A. R. Meyer, D. L. Bajor, C. Clendenin, B. Z. Stanger, E. E. Furth, E. J. Wherry, R. H. Vonderheide, Cancer Immunol Res 2015, 3, 399.
[29] P. Sood, K. B. Thurmond, J. E. Jacob, L. K. Waller, G. O. Silva, D. R. Stewart, D. P. Nowotnik, Bioconjugate Chemistry 2006, 17, 1270.
[30] K. P. Olive, M. A. Jacobetz, C. J. Davidson, A. Gopinathan, D. McIntyre, D. Honess, B. Madhu, M. A. Goldgraben, M. E. Caldwell, D. Allard, K. K. Frese, G. Denicola, C. Feig, C. Combs, S. P. Winter, H. Ireland-Zecchini, S. Reichelt, W. J. Howat, A. Chang, M. Dhara, L. Wang, F. Ruckert, R. Grutzmann, C. Pilarsky, K. Izeradjene, S. R. Hingorani, P. Huang, S. E. Davies, W. Plunkett, M. Egorin, R. H. Hruban, N. Whitebread, K. McGovern, J. Adams, C. Iacobuzio-Donahue, J. Griffiths, D. A. Tuveson, Science 2009, 324, 1457.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A drug delivery vehicle for the delivery of a metal-based drug, wherein said drug delivery vehicle comprises:
a silica nanoparticle wherein:
i) said silica nanoparticle is a solid silica nanoparticle; or:
ii) said silica nanoparticle comprises one or more cavities disposed within said nanoparticle and an outside surface where said one or more cavities are in fluid communication the outside surface of said nanoparticle;
a metal-based drug disposed on the surface of said nanoparticle and/or within said one or more cavities; and
a lipid bilayer disposed on the surface of said nanoparticle where said lipid bilayer fully encapsulates and seals said nanoparticle.

2. The drug delivery vehicle of claim 1, wherein said metal-based drug comprises a metal selected from the group consisting of platinum, palladim, gold, ruthenium, titanium, technetium and rhenium galdolinium, cobalt, lithium, bismuth, iron, calcium, lanthanum, gallium, tin, arsenic, rhodium, copper, zinc, aluminum, lutetium, vanadium, and manganese.

3. The drug delivery vehicle of claim 2, wherein said metal-based drug comprises a metal-based drug selected from the group consisting of a palladium complex drug, a gold complex drug, a ruthenium complex drug, and a titanium complex drug.

4. The drug delivery vehicle of claim 3, wherein said metal-based drug comprises a platinum based chemotherapeutic drug disposed on the surface of said nanoparticle and/or within said one or more cavities of said nanoparticle where said drug comprises a cationic, activated Pt drug.

5. The drug delivery vehicle of claim 4, wherein said metal-based drug comprises a drug selected from the group consisting of 1,2-diaminocyclohexane)platinum(II) (DACHPt), diaminoplatinum(II) (DAPt), ethylenediamine platinum (EDAPt), a cationic form of carboplatin, a cationic form of nedaplatin, a cationic form of heptaplatin, a cationic form of lobaplatin, a cationic form of iproplatin, a cationic form of tetraplatin, a cationic form of satraplatin, a cationic form of triplatin tetranitrate, a cationic form of phenanthriplatin, a cationic form of picoplatin, and a cationic form of setraplatin.

6. The drug delivery vehicle of claim 4, wherein said metal-based drug comprises a drug selected from the group consisting of 1,2-diaminocyclohexane)platinum(II) (DACHPt), diaminoplatinum(II) (DAPt), and ethylenediamine platinum (EDAPt).

7. The drug delivery vehicle of claim 6, wherein said platinum based chemotherapeutic drug comprises 1,2-diaminocyclohexane)platinum(II) (DACHPt).

8. The drug delivery vehicle of claim 6, wherein said platinum based chemotherapeutic drug comprises diaminoplatinum(II) (DAPt).

9. The drug delivery vehicle of claim 6, wherein said platinum based chemotherapeutic drug comprises ethylenediamine platinum (EDAPt).

10. The drug delivery vehicle of claim 3, wherein said metal-based drug comprises a palladium complex.

11. The drug delivery vehicle of claim 10, wherein said metal-based drug comprises trans-[PdCl₂(2-dqmp)] (2-dqmp=diethyl-2-quinolmethylphosphonate or glycoconjugated Pd(II) complex, ([PdCl₂(L) where L=2-deoxy-2-[(2-pyridinylmethylene) amino]-a-D-glucopyranose).

12. The drug delivery vehicle of claim 3, wherein said metal-based drug comprises a gold complex.

13. The drug delivery vehicle of claim 12, wherein said gold complex comprises an Au(III) complex with multidentate ligands.

14. The drug delivery vehicle of claim 13, wherein said Au(III) complex is selected from the group consisting of [Au(en)Cl₂][Cl], [Au(dien)Cl][Cl₂], [Au(cyclam)][ClO₄]₂C1, [Au(terpy)Cl][Cl₂], and [Au(phen)Cl₂][Cl].

15. The drug delivery vehicle of claim 12, wherein said gold complex comprises an Au(III) complex that contains a functionalized bipyridine ligand of the general formula [Au(N—N)Cl₂][PF₆], where N—N is elected from the group consisting of 2,2′-bipyridine; 4,4′-dimethyl-2,2′-bipyridine, 4,4′-dimethoxy-2,2′-bipyridine, and 4,4′-diamino-2,2′-bipyridine.

16. The drug delivery vehicle of claim 12, wherein said gold complex comprises an Au(III) complex of the type [Au(dach)(pn)]Cl₃ where dach is cis-, or trans-1,2-; and S,S-1,2-diaminocyclohexane and pn=1,3-diaminopropane.

17. The drug delivery vehicle of claim 3, wherein said metal-based drug comprises a ruthenium complex.

18. The drug delivery vehicle of claim 17, wherein said ruthenium complex is selected from the group consisting of KP1019, NAMI-A, RAPTA-C, and RAPTA-T.

19. The drug delivery vehicle of claim 3, wherein said metal-based drug comprises a titanocene.

20. The drug delivery vehicle of claim 19, wherein said metal-based drug comprises a titanocene selected from the group consisting of titanocene X, and titanocene Y.

21. The drug delivery vehicle according to any one of claims 1-20, wherein said nanoparticle is a solid nanoparticle.

22. The drug delivery vehicle according to any one of claims 1-20, wherein said nanoparticle comprises one or more cavities disposed within said nanoparticle and an outside surface where said one or more cavities are in fluid communication the outside surface of said nanoparticle.

23. The drug delivery vehicle according to any one of claims 1-22, wherein said drug is disposed on the surface of said nanoparticle.

24. The drug delivery vehicle according to any one of claims 1-23, wherein said drug is disposed within a cavity in said nanoparticle.

25. The drug delivery vehicle according to any one of claims 22-24, wherein said nanoparticle comprise a single cavity.

26. The drug delivery vehicle of claim 25, wherein said nanoparticle comprises a nanobowl.

27. The drug delivery vehicle of claim 25, wherein said nanoparticle comprises a hollow nanosphere.

28. The drug delivery vehicle according to any one of claims 1-24, wherein said nanoparticle comprises a plurality of cavities.

29. The drug delivery vehicle according to any one of claims 1-28, wherein said drug is disposed on the surface of said nanoparticle.

30. The drug delivery vehicle according to any one of claims 1-23, wherein said drug is disposed within a cavity in said nanoparticle.

31. The drug delivery vehicle according to any one of claims 1-30, wherein said nanoparticle comprises a mesoporous silica nanoparticle (MSN), a mesoporous organosilica nanoparticle (MONs), a periodic mesoporous organosilica (PMO) nanoparticle, a solid silica nanoparticle, or a silica thin layer.

32. The drug delivery vehicle of claim 31, wherein said nanoparticle comprises a mesoporous silica nanoparticle (MSN).

33. The drug delivery vehicle according to any one of claims 1-32, wherein said nanoparticle comprises an inorganically doped silica.

34. The drug delivery vehicle of claim 33, wherein said nanoparticle comprises a calcium-, iron-, manganese-, or zirconium-doped silica.

35. The drug delivery vehicle according to any one of claims 1-34, wherein said nanoparticle comprises an imine-doped silica.

36. The drug delivery vehicle according to any one of claims 1-35, wherein said nanoparticle comprises a mesoporous silica/hydroxyapatite (MSNs/HAP) hybrid nanoparticle.

37. The drug delivery vehicle according to any one of claims 1-36, wherein said nanoparticle comprises a cleavable silsesquioxane, or a bridged silsesquioxane (BS).

38. The drug delivery vehicle according to any one of claims 1-32, wherein said nanoparticle is undoped and silica comprising said nanoparticle is not functionalized with a moiety other than a silanol group.

39. The drug delivery vehicle according to any one of claims 1-38, wherein said nanoparticle is functionalized with silanol groups.

40. The drug delivery vehicle according to any one of claims 1-39, wherein said lipid bilayer comprises a phospholipid, and cholesterol (CHOL) and/or a cholesterol derivative.

41. The drug delivery vehicle of claim 40, wherein said lipid bilayer comprises a phospholipid and cholesterol (CHOL).

42. The drug delivery vehicle according to any one of claims 40-41, wherein said phospholipid comprises a saturated fatty acid with a C14-C20 carbon chain, and/or an unsaturated fatty acid with a C14-C20 carbon chain, and/or a natural lipid comprising a mixture of fatty acids with C12-C20 carbon chains.

43. The drug delivery vehicle of claim 42, wherein said phospholipid comprises one or more phospholipids selected from the group consisting of distearoylphosphatidylcholine (DSPC), phosphatidylcholine (DPPC), 1,2-dimyristoleoyl-sn-glycero-3-phosphocholine (DMPC), 1,2-dioleoyl-sn-glycero-3-phosphocholine (DOPC), 1,2-distearoyl-sn-glycero-3-phospho-rac-glycerol (DSPG), 1,2-dipalmitoyl-sn-glycero-3-phosphoglycerol (DPPG), 1,2-dieicosenoyl-sn-glycero-3-phosphocholine, and diactylphosphatidylcholine (DAPC), dipalmitoyl phosphatidylethanolamine, Dioleoyl-N-Glutaryl-L-α-phosphatidylethanolamine (DOPE-Glu), 1-Palmitoyl-2-oleoyl-N-Glutaryl-L-α-phosphatidylethanolamine (POPE-Glu), Dipalmitoyl-N-Glutaryl-L-α-phosphatidylethanolamine (DPPE-Glu), and Distearoyl-N-Glutaryl-L-α-phosphatidylethanolamine (DSPE-Glu).

44. The drug delivery vehicle of claim 42, wherein said phospholipid comprises a natural lipid selected from the group consisting of egg phosphatidylcholine (egg PC), and soy phosphatidylcholine (soy PC).

45. The drug delivery vehicle of claim 42, wherein said phospholipid comprises distearoylphosphatidylcholine (DSPC).

46. The drug delivery vehicle according to any one of claims 40-45, wherein said lipid bilayer comprises an mPEG phospholipid with a phospholipid C14-C18 carbon chain, and a PEG molecular weight ranging from about 350 Da to 5000 Da.

47. The drug delivery vehicle of claim 46, wherein said lipid bilayer comprises dipalmitoyl phosphatidylethanolamine grafted poly(ethylene glycol) (PE-PEG).

48. The drug delivery vehicle of claim 47, wherein said PE-PEG comprises PE-PEG_{2K}.

49. The drug delivery vehicle of claim 47, wherein said PE-PEG comprises PE-PEG_{5K}.

50. The drug delivery vehicle according to any one of claims 45-49, wherein said lipid bilayer comprises DPSC, cholesterol, and PE-PEG.

51. The drug delivery vehicle of claim 50, wherein the ratio of DPSC:cholesterol:PE-PEG ranges from 40-90% DSPC:10%-50% Chol:1%-10% PE-PEG (molar ratio).

52. The drug delivery vehicle of claim 51, wherein said bilayer comprises DSPC:cholesterol:PE-PEG at a molar ratio of about 3:2:0.15 for DSPC, cholesterol, and PE-PEG, respectively.

53. The drug delivery vehicle according to any one of claims 40-52, wherein said lipid bilayer comprises a cholesterol derivative selected from the group consisting of cholesterol hemisuccinate (CHEMS), lysine-based cholesterol (CHLYS), and PEGylated cholesterol (Chol-PEG).

54. The drug delivery vehicle of claim 53, wherein said lipid bilayer comprises CHEMS.

55. The drug delivery vehicle of claim 54, wherein said bilayer comprises CHEMS ranging from about 5% (mol percent) up to about 30% total lipid.

56. The drug delivery vehicle of claim 55, wherein said bilayer comprises about 10% or about 20% CHEMS or about 30% CHEMS or about 40% CHEMS.

57. The drug delivery vehicle of claim 53, wherein said cholesterol derivative is used in place of said cholesterol.

58. The drug delivery vehicle according to any one of claims 1-39, wherein said drug delivery vehicle has an average hydrodynamic diameter ranging from about 30 nm up to about 300 nm, or from about 40 nm up to about 200 nm, or from about 50 up to about 100 nm, or from about 60 nm up to about 90 nm, or from about 70 nm up to about 90 nm, or from about 80 nm up to about 90 nm by DLS.

59. The drug delivery vehicle of claim 58, wherein said drug delivery vehicles have an average hydrodynamic diameter ranging from about 79 nm up to about 86 nm by DLS.

60. The drug delivery vehicle according to any one of claims 1-59, wherein said drug delivery vehicle has an average hydrodynamic diameter ranging from about 30 nm up to about 300 nm, or from about 50 nm up to about 250 nm, or from about 70 nm up to about 200 nm, or from about 90 nm up to about 150 nm, or from about 110 nm up to about 150 nm by cryoEM.

61. The drug delivery vehicle of claim 60, wherein said drug delivery vehicle has an average hydrodynamic diameter ranging from about 136 nm up to about 139 nm by cryoEM.

62. The drug delivery vehicle according to any one of claims 1-61, wherein a plurality of said drug delivery vehicles, in suspension, has a PDI ranging from about 0.050 up to about 0.20, or from about 0.050 up to about 0.1.

63. The drug delivery vehicle of claim 62, wherein a plurality of said drug delivery vehicles, in suspension, has a PDI of about 0.076.

64. The drug delivery vehicle according to any one of claims 1-63, wherein said lipid bilayer ranges in thickness from about 5 to about 12 nm.

65. The drug delivery vehicle of claim 64, wherein said lipid bilayer ranges in thickness from 6 nm to about 7 nm.

66. The drug delivery vehicle according to any one of claims 1-65, wherein said vehicle entraps at least about 50%, or at least about 60%, or at least about 70% said metal-based drug.

67. The drug delivery vehicle of claim 66, wherein said drug delivery provides an EE % of at least about 40%, or at least about 50%, or about 53%.

68. The drug delivery vehicle according to any one of claims 66-67, wherein said drug delivery provides an LC % of at least about 15 wt %, or at least about 20 wt %, or about 21 wt %.

69. The drug delivery vehicle according to any one of claims 1-68, wherein said drug carrier comprises an additional therapeutic agent disposed inside of the nanoparticle or associated with the lipid bilayer.

70. The nanoparticle of claim 69, wherein said drug carrier comprises an additional therapeutic agent disposed inside of the nanoparticle.

71. The nanoparticle of claim 69, wherein said drug carrier comprises an additional therapeutic agent disposed inside of the nanoparticle or disposed within the lipid bilayer or conjugated to the lipid bilayer.

72. The drug delivery vehicle of claim 69, wherein said additional therapeutic agent comprises a second metal-based drug.

73. The drug delivery vehicle of claim 72, wherein said second metal-based drug comprises a metal selected from the group consisting of platinum, palladim, gold, ruthenium, titanium, technetium and rhenium galdolinium, cobalt, lithium, bismuth, iron, calcium, lanthanum, gallium, tin, arsenic, rhodium, copper, zinc, aluminum, lutetium, vanadium, and manganese.

74. The drug delivery vehicle of claim 73, wherein said second metal-based drug comprises a metal-based drug selected from the group consisting of a palladium complex drug, a gold complex drug, a ruthenium complex drug, and a titanium complex drug.

75. The drug delivery vehicle of claim 74, wherein said second metal-based drug comprises a metal-based drug selected from the group consisting trans-[PdCl₂(2-dqmp)] (2-dqmp=diethyl-2-quinolmethylphosphonate, glycoconjugated Pd(II) complex, [PdCl₂(L)] (L=2-deoxy-2-[(2-pyridinylmethylene) amino]-a-D-glucopyranose, [Au(en)Cl₂][Cl], [Au(dien)Cl][Cl₂], [Au(cyclam)][ClO₄]₂C1, [Au(terpy)Cl][Cl₂], [Au(phen)Cl₂][Cl], [Au(N—N)Cl₂][PF₆] where N—N is 2,2′-bipyridine, 4,4′-dimethyl-2,2′-bipyridine, 4,4′-dimethoxy-2,2′-bipyridine, or 4,4′-diamino-2,2′-bipyridine, [Au(dach)(pn)]Cl₃ where dach is cis-, or trans-1,2-, or S,S-1,2-diaminocyclohexane and pn is 1,3-diaminopropane, KP1019, NAMI-A, RAPTA-C, RAPTA-T, titanocene X, and titanocene Y.

76. The drug delivery vehicle of claim 69, wherein said additional therapeutic agent comprises an agent selected from the group consisting of doxorubicin, irinotecan, topotecan, 10-hydroxycamptothecin, belotecan, rubitecan, vinorelbine, LAQ824, vinblastine, vincristine, homoharringtonine, trabectedin, anthracyclines, epirubicin, pirarubicin, daunorubicin, rubidomycin, valrubicin, amrubicin, mitoxantrone, cyclophosphamide, mechlorethamine, temozolomide, 5-fluorouracil, 5′-deoxy-5-fluorouridine, gemcitabine, capecitabine, pazopanib, enzastaurin, vandetanib erlotinib, dasatinib, nilotinib, sunitinib, osimertinib, palbociclib, and ribociclib.

77. The drug delivery vehicle of claim 69, wherein said additional therapeutic agent comprises an inhibitor of the indoleamine 2,3-dioxygenase (IDO) pathway (IDO pathway inhibitor).

78. The drug delivery vehicle of claim 77, wherein said drug carrier, when administered systemically, delivers an amount of an IDO pathway inhibitor to partially or fully inhibit the IDO enzyme or IDO pathway at a cancer site.

79. The drug delivery vehicle according to any one of claims 77-78, wherein said IDO pathway inhibitor comprises an inhibitor of the IDO enzyme.

80. The drug delivery vehicle according to any one of claims 77-79, wherein said IDO pathway inhibitor comprises an inhibitor of the IDO pathway downstream from said IDO enzyme.

81. The drug delivery vehicle according to any one of claims 77-80, wherein said IDO pathway inhibitor comprises an agent selected from the group consisting of of D-1-methyl-tryptophan (indoximod, D-1MT), L-1-methyl-tryptophan (L-1MT), a mixture of D-1MT and L-1MT, 1-methyl-L-tryptophan (L-1MT), methylthiohydantoin-dl-tryptophan (MTH-Trp, Necrostatin), β-carbolines (e.g., 3-butyl-β-carboline), Naphthoquinone-based (e.g., annulin-B), S-allyl-brassinin, S-benzyl-brassinin, N-[2-(Indol-3-yl)ethyl]-S-methyl-dithiocarbamate, N-[2-(benzo[b]thiophen-3-yl)ethyl]-S-methyl-dithiocarbamate, N-[3-(Indol-3-yl)propyl]-S-methyl-dithiocarbamate, S-hexyl-brassinin, N-[2-(indol-3-yl)ethyl]-S-benzyl-dithiocarbamate, N-[2-(indol-3-yl)ethyl]-S[(naphth-2-yl)methyl]-dithiocarbamate, N-[2-(indol-3-yl)ethyl]-S-[(pyrid-3-yl)methyl]-dithiocarbamate, N-[2-(indol-3-yl)ethyl]-S-[(pyrid-4-yl)methyl]-dithiocarbamate, 5-bromo-brassinin, Phenylimidazole-based IDO inhibitors (e.g., 4-phenylimidazole), Exiguamine A, imidodicarbonimidic diamide,N-methyl-N′-9-phenanthrenyl-monohydrochloride (NSC401366), INCB024360 (epacadostat), 1-cyclohexyl-2-(5H-imidazo[5,1-a]isoindol-5-yl)ethanol (GDC-0919), IDO1-derived peptide, NLG919, Ebselen, Pyridoxal Isonicotinoyl Hydrazone, Norharmane, CAY10581, 2-Benzyl-2-thiopseudourea hydrochloride, and 4-phenylimidazole.

82. The drug delivery vehicle of claim 81, wherein said IDO pathway inhibitor comprises 1-methyl-tryptophan.

83. The drug delivery vehicle of claim 82, wherein said IDO pathway inhibitor comprises a “D” enantiomer of 1-methyl-tryptophan (indoximod, 1-MT).

84. The drug delivery vehicle of claim 82, wherein said IDO pathway inhibitor comprises an “L” enantiomer of 1-methyl-tryptophan (L-MT).

85. The drug delivery vehicle according to any one of claims 77-84, wherein said IDO pathway inhibitor, is disposed in a lipid comprising said vesicle and/or conjugated to a lipid comprising said vesicle.

86. The drug delivery vehicle according to any one of claims 77-84, wherein said IDO pathway inhibitor, wherein said IDO inhibitor is conjugated to a component of the lipid bilayer.

87. The drug delivery vehicle of claim 86, wherein said component of a lipid bilayer comprises a moiety selected from the group consisting of a lipid, PHGP, vitamin E, cholesterol, and a fatty acid.

88. The drug delivery vehicle of claim 87, wherein said component of a lipid bilayer comprises cholesterol or a cholesterol derivative.

89. The drug delivery vehicle of claim 69, wherein said drug delivery vehicle comprises a hydrophobic therapeutic agent disposed in the lipid bilayer.

90. The drug delivery vehicle of claim 89, wherein said hydrophobic therapeutic agent comprises paclitaxel.

91. The drug delivery vehicle according to any one of claims 1-90, wherein said drug carrier is conjugated to a moiety selected from the group consisting of a targeting moiety, a fusogenic peptide, and a transport peptide.

92. The drug delivery vehicle of claim 91, wherein said drug carrier is conjugated to a peptide that binds a receptor on a cancer cell or tumor blood vessel.

93. The drug delivery vehicle of claim 92, wherein said drug carrier is conjugated to an iRGD peptide.

94. The drug delivery vehicle of claim 92, wherein said drug carrier is conjugated to a targeting ligand shown in Table 3.

95. The drug delivery vehicle according to any one of claims 91-94, wherein said drug carrier is conjugated to transferrin, and/or ApoE, and/or folate.

96. The drug delivery vehicle according to any one of claims 91-95, wherein said drug carrier is conjugated to a targeting moiety that comprises an antibody that binds to a cancer marker.

97. The drug delivery vehicle of claim 96, wherein said drug carrier is conjugated to a targeting moiety that comprises an antibody that binds a cancer marker shown in Table 2.

98. The drug delivery vehicle according to any one of claims 96-97, wherein said antibody is selected from the group consisting of an intact immunoglobulin, an F(ab)′₂, a Fab, a single chain antibody, a diabody, an affibody, a unibody, and a nanobody.

99. The drug delivery vehicle according to any one of claims 1-98, wherein said drug carriers in suspension are stable for at least 1 month, or at least 2 months, or at least 3 months, or at least 4 months, or at least 5 months, or at least 6 months when stored at 4° C.

100. The drug delivery vehicle according to any one of claims 1-99, wherein said drug delivery vehicle forms a stable suspension on rehydration after lyophilization.

101. The drug delivery vehicle according to any one of claims 1-100, wherein said drug delivery vehicles, show reduced drug toxicity as compared to the corresponding free platinum-based drug.

102. The drug delivery vehicle according to any one of claims 1-101, wherein said drug delivery vehicle has colloidal stability in physiological fluids with pH 7.4 and remains monodisperse to allow systemic biodistribution and is capable of entering a disease site by vascular leakage (EPR effect) or transcytosis.

103. The drug delivery vehicle according to any one of claims 1-102, wherein said carrier is colloidally stable.

104. A pharmaceutical formulation comprising:
drug delivery vehicle according to any one of claims 1-103; and
a pharmaceutically acceptable carrier.

105. The pharmaceutical formulation of claim 104, wherein said formulation is an emulsion, dispersion, or suspension.

106. The pharmaceutical formulation of claim 105, wherein said suspension, emulsion, or dispersion is stable for at least 1 month, or at least 2 months, or at least 3 months, or at least 4 months, or at least 5 months, or at least 6 months when stored at 4° C.

107. The pharmaceutical formulation according to any one of claims 104-106, wherein the nanovesicle drug carriers, and/or the a drug delivery vehicles, and/or the a nanomaterial carriers in said formulation show a substantially unimodal size distribution; and/or show a PDI less than about 0.2, or less than about 0.1.

108. The pharmaceutical formulation according to any one of claims 104-107, wherein said formulation is formulated for administration via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.

109. The pharmaceutical formulation according to any one of claims 104-107, wherein said formulation is a sterile injectable.

110. The pharmaceutical formulation according to any one of claims 104-109, wherein said formulation is a unit dosage formulation.

111. A method of treating a cancer, said method comprising:
administering to a subject in need thereof an effective amount of a drug delivery vehicle according to any one of claims 1-103; and/or
a pharmaceutical formulation according to any one of claims 104-110.

112. The method of claim 111, wherein said method comprises a component of a primary therapy in a chemotherapeutic regimen.

113. The method of claim 111, wherein said method comprises an adjunct therapy in a treatment regime that additionally comprises chemotherapy using another chemotherapeutic agent, and/or surgical resection of a tumor mass, and/or radiotherapy.

114. The method according to any one of claims 111-113, wherein said composition, a nanovesicle drug carrier, a drug delivery vehicle according, and/or nanomaterial carrier is a component in a multi-drug chemotherapeutic regimen.

115. The method according to any one of claims 111-114, wherein said cancer comprises a solid tumor.

116. The method of claim 115, wherein said cancer comprises a cancer selected from the group consisting of gastric cancer, hepatocellular carcinoma, head and neck squamous cell carcinoma, urothelial carcinoma, cervical cancer, non-small cell lung cancer, and broadly for non-respectable solid tumors with high microsatellite instability (MSI-H) or DNA mismatch repair deficiency.

117. The method according to any one of claims 111-114, wherein said cancer comprises pancreatic cancer.

118. The method according to any one of claims 111-114, wherein said cancer comprises colorectal cancer.

119. The method according to any one of claims 111-114, wherein said cancer comprises lung cancer.

120. The method according to any one of claims 111-114, wherein said cancer is a cancer selected from the group consisting of breast cancer, lung cancer, melanoma, pancreas cancer, liver cancer, acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

121. The method according to any one of claims 111-120, wherein said administration in conjunction with an additional therapeutic agent.

122. The method of claim 121, wherein said drug delivery vehicle is administered as a component FOLFIRINOX protocol that additionally includes folinic acid, 5-fluorouracil, and irinotecan.

123. The method of claim 121, wherein said drug delivery vehicle is administered in conjunction with a checkpoint inhibitor.

124. The method of claim 123, wherein said checkpoint inhibitor comprises one or more checkpoint inhibitors selected from the group consisting of a PD-L1 inhibitor, a PD-1 inhibitor, and a CTLA-4 inhibitor.

125. The method of claim 124, wherein said checkpoint inhibitor comprises one or more PD-L1 inhibitors.

126. The method of claim 125, wherein said checkpoint inhibitor comprises an anti-PD-L1 antibody.

127. The method of claim 126, wherein said checkpoint inhibitor comprises an anti-PD-L1 antibody selected from the group consisting of Atezolizumab, Avelumab, Durvalumab, BMS-936559, RG-7446. MPDL3280A, MEDI-4736, and MSB0010718C.

128. The method of claim 125, wherein said checkpoint inhibitor comprises a peptidic PD-L1 inhibitor.

129. The method of claim 128, wherein said PD-L1 inhibitor comprise a moiety selected from the group consisting of AUNP12, CA-170, and BMS-986189.

130. The method according to any one of claims 124-129, wherein said checkpoint inhibitor comprises a PD1 inhibitor.

131. The method of claim 130, wherein said checkpoint inhibitor comprises an anti-PD1 antibody.

132. The method of claim 131, wherein said checkpoint inhibitor comprises an anti-PD1 antibody selected from the group consisting of Nivolumab, Pembrolizumab, Cemiplimab, avelumab, durvalumab, and atezolizumab.

133. The method of claim 130, wherein said checkpoint inhibitor comprises an fc fusion with PD-L2.

134. The method of claim 133, wherein said checkpoint inhibitor comprises AMP224.

135. The method according to any one of claims 124-134, wherein said checkpoint inhibitor comprises CTLA-4 inhibitor.

136. The method of claim 135, wherein said CTLA-4 inhibitor comprises Ipilimumab.

137. The method according to any one of claims 124-136, wherein said checkpoint inhibitor comprises a bispecific antibody that binds to two checkpoint inhibitors, or an antibody that binds to a checkpoint inhibitor attached to a cytokine.

138. The method of claim 137, wherein said checkpoint inhibitor comprises a bispecific antibody that binds to two checkpoint inhibitors.

139. The method of claim 138, wherein said bispecific antibody comprises an antibody that binds to PD-1 attached to an antibody that binds to PD-L1, or an antibody that binds to PD-1 attached to an antibody that binds to CTLA4, or an antibody that binds to PD-L1 attached to an antibody that binds to CTLA4.

140. The method of claim 139, wherein said bispecific antibody comprises an antibody that binds to PD-1 attached to an antibody that binds to CTLA4.

141. The method of claim 137, wherein said checkpoint inhibitor comprises a cytokine attached to an antibody that binds to a checkpoint inhibitor.

142. The method of claim 141, wherein said checkpoint inhibitor comprises a cytokine attached to an antibody selected from the group consisting of anti-PD-1, anti-PD-L1, and CTLA4.

143. The method of claim 142, wherein said checkpoint inhibitor comprises cytokine attached to an anti-PD-1 antibody.

144. The method of claim 143, wherein said checkpoint inhibitor comprises an IL-7 attached to an anti-PD-1 antibody.

145. The method according to any one of claims 111-144, wherein said administration is via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.

146. The method according to any one of claims 111-144, wherein said administration comprises systemic administration via injection or cannula.

147. The method according to any one of claims 111-144, wherein said administration is administration to an intra-tumoral or peri-tumoral site.

148. The method according to any one of claims 111-147, wherein said mammal is a human.

149. The method according to any one of claims 111-147, wherein said mammal is a non-human mammal.

150. A method of loading silica nanoparticles with a metal-based drug, said method comprising:
contacting said silica nanoparticles with a cationic form of said metal-based drug at a basic pH to form a mixture of said silica nanoparticles and metal-based drug; and
applying energy to said mixture where said application of energy enhances loading of said metal-based drug into said silica nanoparticles.

151. The method of claim 150, wherein said metal-based drug comprises a metal selected from the group consisting of platinum, palladim, gold, ruthenium, titanium, technetium and rhenium galdolinium, cobalt, lithium, bismuth, iron, calcium, lanthanum, gallium, tin, arsenic, rhodium, copper, zinc, aluminum, lutetium, vanadium, and manganese.

152. The method of claim 151, wherein said metal-based drug comprises a metal-based drug selected from the group consisting of a palladium complex drug, a gold complex drug, a ruthenium complex drug, and a titanium complex drug.

153. The method of claim 152, wherein said contacting comprises contacting said silica nanoparticles with a cationic activated platinum-based drug at a basic pH to form a mixture of said silica nanoparticles and platinum-based drug.

154. The method of claim 153, wherein said activated platinum-based drug comprises a drug selected from the group consisting of 1,2-diaminocyclohexane)platinum(II) (DACHPt), diaminoplatinum(II) (DAPt), and ethylenediamine platinum (EDAPt).

155. The method of claim 154, wherein said platinum based chemotherapeutic drug comprises (1,2-diaminocyclohexane)platinum(II) (DACHPt).

156. The method of claim 154, wherein said platinum based chemotherapeutic drug comprises diaminoplatinum(II) (DAPt).

157. The method of claim 154, wherein said platinum based chemotherapeutic drug comprises ethylenediamine platinum (EDAPt).

158. The method of claim 150, wherein said activated platinum-based drug comprises a cationic version of a drug selected from the group consisting of carboplatin, nedaplatin, heptaplatin, lobaplatin, iproplatin, tetraplatin, satraplatin, triplatin tetranitrate, phenanthriplatin, picoplatin, and setraplatin.

159. The method of claim 152, wherein said contacting comprises contacting said silica nanoparticles with a cationic version of a drug selected from the group consisting trans-[PdCl₂(2-dqmp)] (2-dqmp=diethyl-2-quinolmethylphosphonate, glycoconjugated Pd(II) complex, [PdCl₂(L)] (L=2-deoxy-2-[(2-pyridinylmethylene) amino]-a-D-glucopyranose, [Au(en)Cl₂][Cl], [Au(dien)Cl][Cl₂], [Au(cyclam)][ClO₄]₂C1, [Au(terpy)Cl][Cl₂], [Au(phen)Cl₂][Cl], [Au(N—N)Cl₂][PF₆] where N—N is 2,2′-bipyridine, 4,4′-dimethyl-2,2′-bipyridine, 4,4′-dimethoxy-2,2′-bipyridine, or 4,4′-diamino-2,2′-bipyridine, [Au(dach)(pn)]Cl₃ where dach is cis-, or trans-1,2-, or S,S-1,2-diaminocyclohexane and pn is 1,3-diaminopropane, KP1019, NAMI-A, RAPTA-C, RAPTA-T, titanocene X, and titanocene Y.

160. The method according to any one of claims 150-159, wherein said pH ranges from about pH 8 up to about pH 9.

161. The method of claim 160, wherein said pH is about pH 8.5.

162. The method according to any one of claims 150-161, wherein the ratio of drug to silica nanoparticle ranges from about 0.1:about 2 (w/w drug:NP), or from about 0.2:1.5 (w/w drug:NP), or from about 0.2:about 1 (w/w drug:NP).

163. The method of claim 162, wherein the ratio of drug to silica nanoparticle is about 0.4:1 (w/w drug:NP).

164. The method according to any one of claims 150-163, wherein said method further comprises encapsulating said nanoparticles within lipid bilayers.

165. The method of claim 164, wherein said encapsulating comprises using ethanol exchange.

166. The method according to any one of claims 150-165, wherein said method produces a nanoparticle drug delivery vehicle according to any one of claims 1-103.
